(19) United States
(12) Patent Application Publication
(10) Pub. No.: US 2026/0224721 A1
(43) Pub. Date: Aug. 6, 2026

(54) ANTI-CD70 ANTIBODY-DRUG CONJUGATES

(71) Applicant: Ambrx, Inc., La Jolla, CA (US)

(72) Inventors: Lillian SKIDMORE, La Jolla, CA (US); Nickolas KNUDSEN, La Jolla, CA (US); Yingchun LU, La Jolla, CA (US); Mysore RAMPRASAD, La Jolla, CA (US); Ji Young KIM, La Jolla, CA (US)

(21) Appl. No.: 19/147,964

(22) PCT Filed: Jan. 16, 2024
(86) PCT No.: PCT/US2024/011683

§ 371 Date: Jul. 14, 2025

Related U.S. Application Data
(60) Provisional application No. 63/480,091, filed on Jan. 16, 2023.
(60) Provisional application No. 63/492,848, filed on Mar. 29, 2023.

(51) Int. Cl. A61K 47/68; A61K 9/19; A61K 45/06; A61K 47/22; A61K 47/26; A61P 35/00; C07K 16/28

(52) U.S. Cl. CPC A61K 47/68031; A61K 9/19; A61K 45/06; A61K 47/22; A61K 47/26; A61K 47/6849; A61K 47/6889; A61P 35/00; C07K 16/2875

(57) ABSTRACT

This invention relates to anti-CD70 antibodies and antibody drug conjugates comprising at least one non-naturally-encoded amino acid. Disclosed herein are anti-CD70 antibodies with one or more non-naturally-encoded amino acids and further disclosed are antibody drug conjugates wherein the anti-CD70 antibodies of the invention are conjugated to one or more toxins. Further disclosed are methods for using such non-naturally-encoded amino acid-containing antibody drug conjugates, including therapeutic, diagnostic, and other biotechnology uses.

### CROSS-REFERENCE TO RELATED APPLICATIONS

This application claims the benefit of U.S. Provisional Patent Application No. 63/480,091 filed Jan. 16, 2023, and U.S. Provisional Patent Application No. 63/492,848, filed Mar. 29, 2023, each of which is entirely incorporated herein by reference.

### REFERENCE TO A SEQUENCE LISTING

The instant application contains a Sequence Listing which has been submitted electronically in XML format and is hereby incorporated by reference in its entirety. Said XML copy, created on Jan. 14, 2024, is named AMBX_0242_00_PCT.xml and is 14,309 bytes in size.

### FIELD OF THE INVENTION

This invention relates to anti-CD70 antibodies and antibody drug conjugates comprising at least one non-naturally-encoded amino acid. Disclosed herein are anti-CD70 antibodies with one or more non-naturally encoded amino acids and further disclosed are antibody drug conjugates wherein the anti-CD70 antibodies of the invention are conjugated to one or more cytotoxic drug-linker moieties. Further disclosed are methods for using such non-natural amino acid antibody drug conjugates, including therapeutic, diagnostic, and other biotechnology uses.

### BACKGROUND OF THE INVENTION

Cluster of differentiation 70 (CD70) is a member of the tumor necrosis factor superfamily and the ligand for CD27 (Goodwin, R. G. et al., Cell, 73:447-456 (1993); Hintzen, R. Q. et al., Int Immunol, 6:477-480 (1994)). CD70 was first identified in activated T- and B-lymphocytes. The binding of CD70 to CD27 on activated lymphocytes signals the co-stimulation of T cells, B cells and natural killer (NK) cells (Grewal, I. S., Expert Opin Ther Targets, 12(3):341-351 (2008); Borst, J. et al., Curr Opin Immunol., 17(3):275-281 (2005)) and regulates cell differentiation and T-helper ½ switching (Wajant, H., Expert Opin Ther Targets, 20(8):959-973 (2016)).

The primary amino acid sequence of CD70 predicts a transmembrane type II protein with its carboxyl terminus exposed to the outside of cells and its amino terminus found in the cytosolic side of the plasma membrane. Human CD70 is composed of a 20 amino acid cytoplasmic domain, an 18 amino acid transmembrane domain, and a 155 amino acid extracytoplasmic domain with two potential N-linked glycosylation sites (Bowman et al., J Immunol, 152:1756-1761 (1994); Goodwin et al., Cell, 73:447-456 (1993)).

CD70 expression has been reported in different types of cancers including lymphomas, carcinomas and tumors of neural origin. In malignant B cells, 71% of diffuse large B-cell lymphomas, 33% of follicle center lymphomas, 25% of mantle lymphomas and 50% of B-CLL have been reported to express CD70 (Lens et al., 1999, Br J Haematol, 106:491-503). CD70 has also been detected on brain tumor cells, especially glioma cell lines, solid human gliomas, and meningiomas (Held-Feindt and Mentlein, Int J Cancer, 98:352-56 (2002); Wischlusen et al., Can Res, 62:2592-2599 (2002)). CD70 is frequently expressed in renal cell carcinoma (RCC; 87%) and non-Hodgkin's lymphoma (NHL; 77%) (Tannir, N. M. et al., Invest New Drugs, 32(6):1246-1257 (2014)), but minimally expressed in normal tissues (Nakae, R. et al., Am J Obstet Gynecol., 224(2):197 (2021)).

Anti-CD70 antibodies and antibody-drug conjugates (ADCs), and methods of making and using them to treat diseases such as cancer, are disclosed in WO2013/192360A1, the entire contents of which are hereby incorporated by reference in their entirety.

Multiple clinical trials evaluating anti-CD70 agents (e.g., antibodies with enhanced antibody-dependent cell-mediated cytotoxicity, ADCs, and chimeric antigen receptor (CAR) T-cell therapy) are being examined in malignancies showing high CD70 expression. Previous studies have shown that anti-CD70 monoclonal antibodies (mAb) and anti-CD70 ADCs exhibit anti-tumor effects in xenograft models of CD70 malignant diseases, such as lymphoma, NHL and RCC (Israel, B. F. et al., Mol Cancer Ther., 4(12):2037-2044 (2005); Law, C. L. et al., Cancer Res., 66:2328-2337 (2006); McEarchern, J. A. et al., Blood, 109(3):1185-92 (2007)).

Based on the results of preclinical studies, two separate Phase 1 studies of SGN-75 (anti-CD70 mAb conjugated to maleimidocaproyl-monomethyl auristatin F (MMAF)) in patients with CD70-positive relapsed/refractory NHL or metastatic RCC were conducted; however, SGN-75 exhibited modest effects on these diseases with some intolerable side effects (Tannir, N. M. et al., Invest New Drugs, 32(6):1246-1257 (2014)). An additional anti-CD70 ADC, SGN-CD70A (anti-CD70 mAb conjugated to a pyrrolobenzodiazepine dimer), was introduced into Phase 1 clinical trials (Pal, S. K. et al., Cancer, 125(7):1124-1132 (2019)), but the SGN-CD70A Phase 1 study was discontinued in 2018.

Thus, there remains a need for anti-CD70 ADCs that are constructed in such a manner so as to be capable of exerting a clinically useful cytotoxic, cytostatic, or immunosuppressive effect on CD70-expressing cells, particularly without exerting undesirable effects on non-CD70-expressing cells. Such ADCs would be useful therapeutic agents against cancers that express CD70 or immune disorders that are mediated by CD70-expressing cells. The present invention provides such ADCs for use in immunology and oncology.

### SUMMARY OF THE INVENTION

Disclosed herein are antibody-drug conjugates (ADCs) comprising anti-CD70 antibodies joined to drug-linker moieties through one or more non-natural amino acids, and methods for making such ADCs. Also described are methods of treating a disease or condition via administration of an ADC of the present disclosure, or a composition comprising an ADC of the present disclosure.

In some general aspects, the present disclosure provides an antibody-drug conjugate (ADC) comprising:
a drug-linker group having the structure:

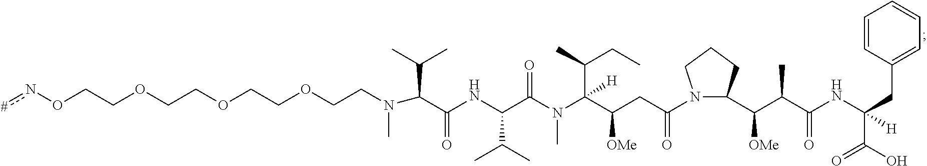

an anti-CD70 antibody or fragment thereof comprising one or more heavy chains; wherein:
at least one member of the one or more heavy chains comprises a sequence comprising SEQ ID NO: 3,
[symbol] represents a single bond or a double bond, and
#represents a connection to the anti-CD70 antibody or fragment thereof.

In some embodiments, the drug-linker group is one or more drug linker groups.

In some other general aspects, there is provided an ADC comprising:
one or more drug-linker groups having the structure:

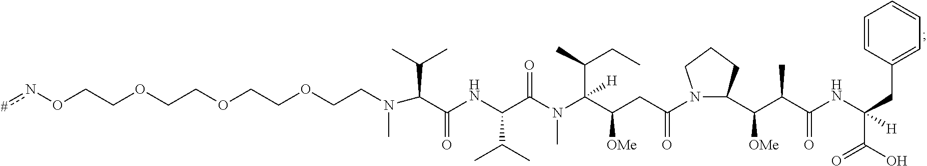

and an anti-CD70 antibody or fragment thereof comprising one or more heavy chains, wherein at least one of the one or more heavy chains comprises an amino acid sequence containing a first non-naturally encoded amino acid, wherein the amino acid sequence is selected from the group consisting of SEQ ID NO: 3, 4 and 5;
wherein:
each [symbol] represents a single bond or a double bond that covalently joins one of the one or more drug-linker groups to the anti-CD70 antibody or fragment thereof, and each #represents a site of connection to the anti-CD70 antibody or fragment thereof.

In some embodiments, at least one of the one or more heavy chains comprises the amino acid sequence of SEQ ID NO: 3. In some embodiments, the heavy chain amino acid sequence is SEQ ID NO: 3.

In some other embodiments, at least one of the one or more heavy chains comprises the amino acid sequence of SEQ ID NO: 4.

In some other embodiments, at least one of the one or more heavy chains comprises the amino acid sequence of SEQ ID NO: 5.

In some embodiments, the ADC further comprises one or more light chains. In some embodiments, at least one of the one or more light chains comprises an amino acid sequence that shares at least 90% identity with SEQ ID NO: 2, 6, 7, 8 or 9.

In some embodiments, at least one of the one or more light chains comprises the amino acid sequence of SEQ ID NO: 2. In some embodiments, at least one of the one or more light chains is the amino acid sequence of SEQ ID NO: 2. In some embodiments, each of the one or more light chains is the amino acid sequence of SEQ ID NO: 2.

In some other embodiments, at least one of the one or more light chains comprises an amino acid sequence containing a second non-naturally encoded amino acid, wherein the amino acid sequence is selected from the group consisting of SEQ ID NO: 6, 7, 8 and 9.

In some embodiments, the anti-CD70 antibody or fragment thereof comprises two heavy chains, wherein one heavy chain comprises the first non-naturally encoded amino acid, and wherein the other heavy chain comprises a third non-naturally encoded amino acid. In some embodiments, each of the one heavy chain and the other heavy chain comprises the amino acid sequence of SEQ ID NO: 3. In some embodiments, each of the one heavy chain and the other heavy chain is SEQ ID NO: 3.

In some embodiments, each [symbol] represents a double bond. In some embodiments, each [symbol] covalently joins one of the one or more drug-linker groups to one non-naturally encoded amino acid of the anti-CD70 antibody or fragment thereof.

In some embodiments, each of the first, second and third non-naturally encoded amino acids is para-acetyl-L-phenylalanine.

In some embodiments, the ADC is an ADC of Formula (I):

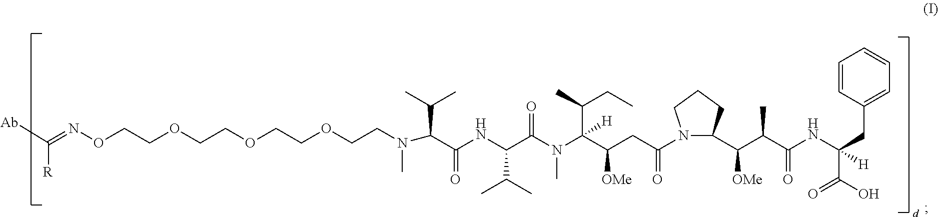

wherein:
the ADC comprises one or more drug-linker groups;
Ab is the anti-CD70 antibody or fragment thereof;
each R is independently unsubstituted C₁-C₈ alkyl;
d is an integer of 1-10; and
each of the one or more drug-linker groups has the following structure:

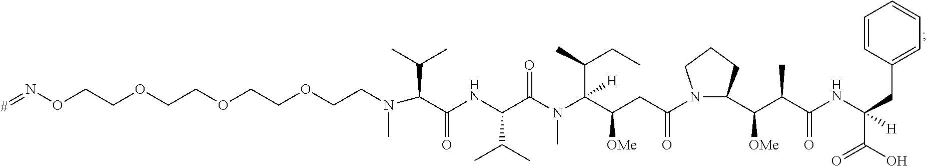

wherein each #represents a site of connection to the anti-CD70 antibody or fragment thereof.

In some embodiments, d is 1, 2, 3 or 4.

In some embodiments, each R is methyl.

In some embodiments, the anti-CD70 antibody or fragment thereof comprises two heavy chains, wherein each heavy chain comprises the amino acid sequence of SEQ ID NO: 3. In some embodiments, each heavy chain amino acid sequence is SEQ ID NO: 3.

In some embodiments, the anti-CD70 antibody or fragment thereof comprises two light chains, wherein each light chain comprises the amino acid sequence of SEQ ID NO: 2. In some embodiments, each light chain amino acid sequence is SEQ ID NO: 2.

In some embodiments, the anti-CD70 antibody or fragment thereof is humanized.

In some embodiments, the anti-CD70 antibody or fragment thereof is a humanized monoclonal antibody comprising two heavy chains and two light chains, wherein each heavy chain comprises the amino acid sequence of SEQ ID NO: 3, and each light chain comprises the amino acid sequence of SEQ ID NO: 2. In some embodiments, the anti-CD70 antibody or fragment thereof is a humanized monoclonal antibody comprising two heavy chains and two light chains, wherein each heavy chain amino acid sequence is SEQ ID NO: 3, and each light chain amino acid sequence is SEQ ID NO: 2.

In some embodiments, the amino acid sequence of SEQ ID NO: 3 comprises one non-naturally-encoded amino acid, wherein the one non-naturally encoded amino acid is para-acetyl-L-phenylalanine.

In some embodiments, d is 2.

In some embodiments, the anti-CD70 antibody or fragment thereof is a humanized anti-CD70 monoclonal antibody comprising two heavy chains, two light chains, and two non-naturally encoded amino acids, wherein:
each of the two heavy chains comprises the amino acid sequence of SEQ ID NO: 3, wherein SEQ ID NO: 3 comprises one non-naturally encoded amino acid, wherein the one non-naturally encoded amino acid in SEQ ID NO: 3 is para-acetyl-L-phenylalanine at position A114 (Kabat numbering) of SEQ ID NO: 3; and
each of the light chains comprises the amino acid sequence of SEQ ID NO: 2;
each R is methyl;
the one or more drug-linker groups is two drug-linker groups; and
d is 2;
   wherein each of the two drug-linker groups is connected to the para-acetyl-L-phenylalanine at position A114 of SEQ ID NO: 3, thereby connecting each of the two drug-linker groups to the humanized anti-CD70 monoclonal antibody.

In some embodiments, each heavy chain amino acid sequence is SEQ ID NO: 3. In some embodiments, each light chain amino acid sequence is SEQ ID NO: 2.

In some embodiments, the non-naturally encoded amino acid is one non-naturally encoded amino acid.

In some embodiments, the ADC of the present disclosure is the ADC of FIG. 1. In some general aspects, the present disclosure provides a composition comprising an ADC of the present disclosure. In some embodiments, the composition comprising the ADC further comprises an additional therapeutic agent. In some embodiments, the additional therapeutic agent is an immunotherapeutic agent, chemotherapeutic agent, hormonal agent, antitumor agent, immunostimulatory agent or immunomodulator, or a combination thereof. In some embodiments, the additional therapeutic agent is a checkpoint inhibitor, CD70 kinase inhibitor, cyclin-dependent kinase inhibitor, tyrosine kinase inhibitor, small-molecule kinase inhibitor, hypomethylating agent or platinum-based therapeutic, or a combination thereof. In some embodiments, the composition comprising the ADC and optionally further comprising an additional therapeutic agent is a pharmaceutical composition, and the pharmaceutical composition further comprises a pharmaceutically acceptable excipient.

In some other general aspects, the present disclosure provides a method of killing a cell, comprising contacting a cell with an effective amount of an ADC of the present disclosure, or a composition comprising an effective amount of an ADC of the present disclosure, or more particularly, a formulation comprising an effective amount of an ADC of the present disclosure. In some embodiments, cell is a tumor or cancer cell. In some embodiments, the tumor or cancer cell is a renal cell carcinoma (RCC) tumor or cancer cell.

In some other general aspects, the present disclosure provides a method of treating a disease or condition in a subject in need thereof, the method comprising administering to the subject an effective amount of an ADC of the present disclosure, a composition comprising an effective amount of an ADC of the present disclosure, or a formulation comprising an effective amount of an ADC of the present disclosure. In some embodiments, the disease or condition is a tumor or cancer. In some embodiments, the tumor or cancer is a solid tumor. In some other embodiments, the tumor or cancer is a hematologic cancer. In some embodiments, the hematologic cancer is lymphoma, multiple myeloma or leukemia. In some embodiments, the tumor or cancer is kidney cancer, brain cancer, breast cancer, Burkitt's lymphoma, ovarian cancer, gastric cancer, gastroesophageal junction adenocarcinoma, cervical cancer, uterine cancer, endometrial cancer, testicular cancer, prostate cancer, colorectal cancer, esophageal cancer, bladder cancer, lung cancer, non-small cell lung cancer, urothelial carcinoma, cholangiocarcinoma, colon biliary tract cancer, pancreatic cancer, renal cell cancer, nasopharyngeal cancer, mantle cell lymphoma, multiple myeloma, non-Hodgkin's lymphoma, Hodgkin's lymphoma or acute myeloid leukemia. In some embodiments, the tumor or cancer is renal cell cancer, brain cancer, multiple myelomamantle cell lymphoma or lung cancer. In some embodiments, the tumor or cancer is renal cell cancer. In some embodiments, the renal cell cancer is clear cell renal cell carcinoma. In some embodiments, the tumor or cancer is a multi-drug resistant tumor or cancer. In some embodiments, the method comprises treating the subject with radiotherapy. In some embodiments, the method comprises treating the subject with an effective amount of an additional therapeutic agent. In some embodiments, the additional therapeutic agent is a chemotherapeutic agent, a hormonal agent, an antitumor agent, an immunostimulatory agent, an immunomodulator or an immunotherapeutic agent; or a combination thereof. In some embodiments, the additional therapeutic agent is a checkpoint inhibitor, CD70 kinase inhibitor, cyclin-dependent kinase inhibitor, tyrosine kinase inhibitor, small-molecule kinase inhibitor, hypomethylating agent or platinum-based therapeutic; or a combination thereof. In some embodiments, the additional agent is a checkpoint inhibitor, wherein the checkpoint inhibitor is a PD-1 inhibitor. In some embodiments, the PD-1 inhibitor is AMP-224, atezolizumab, avelumab, BMS-936558, BMS-936559, CT-001, durvalumab, MEDI0680, nivolumab, PDR001, pembrolizumab, pidilizumab and REGN2810. In some embodiments, the PD-1 inhibitor is an antibody. In some embodiments, the PD-1 inhibitor is pembrolizumab. In some embodiments, the method improves or optimizes cancer cell kill. In some embodiments, the method delays progression or recurrence of the tumor or cancer. In some embodiments, the tumor or cancer is a CD70-expressing cancer. In some embodiments, the CD70-expressing cancer has at least about 5,000 CD70/cell. In some embodiments, the CD70-expressing cancer has at least about 10,000 CD70/cell. In some embodiments, the CD70-expressing cancer has at least about 15,000 CD70/cell. In some embodiments, the subject, cancer or tumor is resistant or refractory to prior standard therapies. In some embodiments, the subject has cancer metastases from a same or different cancer. In some other embodiments, the disease or condition being treated is myelodysplastic syndrome. In some embodiments, the subject being treated for myelodyplastic syndrom is undergoing treatment with, or has previously been treated with, a hypomethylating agent.

In some embodiments, the subject being treated for a disease or condition including a tumor or cancer is a human subject. In some embodiments, the human subject is an adult.

In some embodiments, an effective amount of ADC for administration to a human subject is a dose within a range of about 0.05 mg/kg to about 10 mg/kg of the human subject, or any value in between. In some embodiments, the effective amount of the ADC is a dose within a range of about 0.05 mg/kg to about 2 mg/kg of the human subject, or any value in between. In some embodiments, the effective amount of the ADC is a dose of about 0.05 mg/kg, about 0.1 mg/kg, about 0.12 mg/kg, about 0.14 mg/kg, about 0.16 mg/kg, about 0.18 mg/kg, about 0.2 mg/kg, about 0.22 mg/kg, about 0.24 mg/kg, about 0.26 mg/kg, about 0.28 mg/kg, about 0.3 mg/kg, about 0.32 mg/kg, about 0.34 mg/kg, about 0.36 mg/kg, about 0.38 mg/kg, about 0.4 mg/kg, about 0.42 mg/kg, about 0.44 mg/kg, about 0.46 mg/kg, about 0.48 mg/kg, about 0.5 mg/kg, about 0.52 mg/kg, about 0.54 mg/kg, about 0.56 mg/kg, about 0.58 mg/kg, about 0.6 mg/kg, about 0.62 mg/kg, about 0.64 mg/kg, about 0.66 mg/kg, about 0.68 mg/kg, about 0.7 mg/kg, about 0.72 mg/kg, about 0.74 mg/kg, about 0.76 mg/kg, about 0.78 mg/kg, about 0.8 mg/kg, about 0.82 mg/kg, about 0.84 mg/kg, about 0.86 mg/kg, about 0.88 mg/kg, about 0.9 mg/kg, about 0.92 mg/kg, about 0.94 mg/kg, about 0.96 mg/kg, about 0.98 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg or about 2 mg/kg of the human subject. In some other embodiments, the effective amount of the ADC is a dose within a range of about 2 mg/kg to about 5 mg/kg of the human subject, or any value in between. In some embodiments, the effective amount of the ADC is a dose of about 2 mg/kg, about 2.2 mg/kg, about 2.4 mg/kg, about 2.6 mg/kg, about 2.8 mg/kg, about 3 mg/kg, about 3.2 mg/kg, about 3.4 mg/kg, about 3.6 mg/kg, about 3.8 mg/kg, about 4 mg/kg, about 4.2 mg/kg, about 4.4 mg/kg, about 4.6 mg/kg, about 4.8 mg/kg or about 5 mg/kg of the human subject. In some other embodiments, the effective amount of the ADC is a dose within a range of about 5 mg/kg to about 10 mg/kg of the human subject, or any value in between. In some embodiments, the effective amount of an ADC is a dose of about 5 mg/kg, about 5.2 mg/kg, about 5.4 mg/kg, about 5.6 mg/kg, about 5.8 mg/kg, about 6 mg/kg, about 6.2 mg/kg, about 6.4 mg/kg, about 6.6 mg/kg, about 6.8 mg/kg, about 7 mg/kg, about 7.2 mg/kg, about 7.4 mg/kg, about 7.6 mg/kg, about 7.8 mg/kg, about 8 mg/kg, about 8.2 mg/kg, about 8.4 mg/kg, about 8.6 mg/kg, about 8.8 mg/kg, about 9 mg/kg, about 9.2 mg/kg, about 9.4 mg/kg, about 9.6 mg/kg, about 9.8 mg/kg or about 10 mg/kg of the human subject.

In some embodiments, the ADC, composition or formulation is administered orally, intradermally, intratumorally, intravenously or subcutaneously. In some embodiments, the ADC, composition or formulation is administered intravenously.

In some embodiments, the administering of the effective amount of the ADC is performed on a dosing schedule. In some embodiments, the dosing schedule is once every 1, 2, 3, 4, 5 or 6 weeks. In some embodiments, the dosing schedule is once every two weeks. In some embodiments, the dosing schedule is once every three weeks. In some embodiments, the dosing schedule is once every four weeks. In some embodiments, the dosing schedule is more than once within a three-week cycle. In some embodiments, the administering is at least once every four weeks for at least about eight weeks. In some embodiments, the administering is once every three weeks for at least about eight weeks.

In some other general aspects, the present disclosure provides an isolated anti-CD70 antibody or fragment thereof comprising an amino acid sequence selected from the group consisting of the sequences listed in Table 1. In some embodiments, the isolated anti-CD70 antibody or fragment thereof comprises a heavy chain, wherein the heavy chain comprises the amino acid sequence of SEQ ID NO: 3. In some embodiments, the heavy chain amino acid sequence is SEQ ID NO: 3. In some embodiments, the isolated anti-CD70 antibody or fragment thereof comprises a light chain, wherein the light chain comprises the amino acid sequence of SEQ ID NO: 2. In some embodiments, the light chain amino acid sequence is SEQ ID NO: 2. In some embodiments, the isolated anti-CD70 antibody or fragment thereof comprises a heavy chain, wherein the heavy chain comprises the amino acid sequence of SEQ ID NO: 4. In some embodiments, the isolated anti-CD70 antibody or fragment thereof comprises a heavy chain, wherein the heavy chain comprises the amino acid sequence of SEQ ID NO: 5. In some embodiments, the isolated anti-CD70 antibody or fragment thereof comprises a heavy chain and a light chain, wherein the heavy chain comprises the amino acid sequence of SEQ ID NO: 3, and wherein the light chain comprises the amino acid sequence of SEQ ID NO: 2. In some embodiments, the isolated anti-CD70 antibody or fragment thereof comprises a heavy chain and a light chain, wherein the heavy chain amino acid sequence is SEQ ID NO: 3, and the light chain amino acid sequence is SEQ ID NO: 2.

In some other general aspects, the present disclosure provides a nucleic acid encoding any one of SEQ ID NOs: 1 to 9.

In some other general aspects, the present disclosure provides a vector comprising a nucleic acid encoding any one of SEQ ID NOs: 1 to 9.

In some other general aspects, the present disclosure provides use of an ADC of any one of claims 1 to 20, or an antibody or fragment thereof comprising an amino acid sequence listed in Table 1, in the manufacture of a medicament for the treatment of a disease or condition in a subject.

In some other aspects, the present disclosure provides a formulation comprising an ADC of the present disclosure. In some embodiments, the ADC concentration in the formulation is within a range of about 5 mg/mL to about 25 mg/mL. In some embodiments, the formulation further comprises a buffer, sucrose and a surfactant. In some embodiments, the formulation comprises the ADC and a histidine buffer; sucrose; and polysorbate 80. In some embodiments, the formulation has a pH within a range of about 5.4 to about 6.4. In some embodiments, the formulation has a pH within a range of about 5.2 to about 6.2. In some embodiments, the formulation pH is no greater than about 6. In some embodiments, the formulation pH is less than 6.

In some embodiments, the formulation is a liquid formulation. In some embodiments, liquid formulation of the present disclosure can be stored at room temperature. In some other embodiments, the liquid formulation can be stored frozen.

In some other embodiments, the formulation is a lyophilized drug product. In some embodiments, the lyophilized drug product, when reconstituted with a diluent, provides a reconstituted solution comprising the ADC at a concentration within a range of about 5 mg/mL to about 25 mg/mL. In some embodiments, the reconstituted solution further comprises L-histidine buffer at a concentration within a range of about 10 mM to about 50 mM; sucrose at a concentration within a range of about 1% (w/v) to about 20% (w/v); and polysorbate 80 at a concentration within a range of about 0.01% (w/v) to about 0.1% (w/v). In some embodiments, the reconstituted solution has a pH within a range of about 5.4 to about 6.4. In some embodiments, the reconstituted solution has a pH within a range of about 5.2 to about 6.2. In some embodiments, the reconstituted solution pH is no greater than about 6. In some embodiments, the reconstituted solution pH is less than 6. In some embodiments, the diluent is water.

In some other aspects, the present disclosure provides a formulation comprising an ADC of the present disclosure from about 5 mg/mL to about 15 mg/mL; histidine buffer from about 15 mM to about 25 mM; sucrose from about 5% (w/v) to about 15% (w/v); and polysorbate 80 from about 0.01% (w/v) to about 0.05% (w/v); wherein the formulation pH is from about 5.4 to about 6.0. In some embodiments, the formulation comprises the ADC at about 10 mg/mL; histidine buffer at about 20 mM, sucrose at about 8% (w/v); and polysorbate 80 at about 0.02% (w/v); wherein the formulation pH is about 5.7.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein, which will be limited only by the appended claims.

### INCORPORATION BY REFERENCE

All publications and patents mentioned herein are incorporated herein by reference in their entirety to the same extent as if each individual publication or patent application was specifically and individually indicated to be incorporated by reference, and for the purpose of describing and disclosing, for example, the chemistry, chemical syntheses, compositions and other methodologies that are described in the publications, which might be used in connection with the presently described inventions. The publications discussed herein are provided solely for their disclosure prior to the filing date of the present application.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary antibody-drug conjugate (ADC) of the invention containing a drug-linker (cytotoxic tubulin inhibitor AS269) site-specifically conjugated to an anti-CD70 monoclonal antibody (one drug-linker payload per heavy chain).

FIG. 2 shows a graphical illustration of an HPLC chromatogram of unconjugated anti-CD70 mAb (DAR=0) and anti-CD70-AS269 ADC (DAR=1 and DAR=2) after a conjugation reaction time of 24 hours (see Example 5).

FIG. 3 shows a graphical illustration of anti-CD70-AS269 ADC stability in mouse plasma over 7 days at 37° C. incubation (see Example 6).

FIG. 4 shows a graphical illustration of the Intact ADC (DAR=2) and Total Antibody (unconjugated and conjugated species) concentrations of anti-CD70-AS269 ADC, as well as the Total Antibody (TA) concentration of Unconjugated mAb, in the blood of mice following single administration of 1 mg/kg anti-CD70-AS269 ADC (see Example 7).

FIG. 5 shows a graphical illustration of mean tumor volumes in mice following a single administration of unconjugated anti-CD70 mAb or anti-CD70-AS269 ADC in a 786-O S3 renal cell carcinoma (RCC) xenograft model (see Example 8).

FIG. 6 shows a graphical illustration of mean tumor volumes in mice treated with sunitinib (QD×35) or anti-CD70-AS269 ADC (QW×5) in a 786-O S3 RCC xenograft model (see Example 9).

FIG. 7 shows a graphical illustration of tumor volume in mice treated with three different doses of anti-CD70-AS269 ADC (“aCD70-AS269”; QW×5) in a Caki-1 RCC xenograft model (see Example 10).

FIG. 8 shows a dot plot of final tumor volumes from mice treated with three different doses of anti-CD70-AS269 ADC (“aCD70-AS269”; QW×5) as measured following Caki-1 tumor transplantation (see Example 11).

FIG. 9 shows a graphical illustration of viability of MDR-positive 786-O cells treated with various concentrations of anti-CD70-AS269 ADC (“aCD70-AS269”), alone or in the presence of verapamil or elacridar, or anti-CD70-MMAE ADC, alone or in the presence of verapamil or elacridar (see Example 12).

FIG. 10 shows the serum Ig lambda (released by U266 cells) concentrations correlated with tumor burden in mice treated with a single dose of anti-CD70-AS269 ADC (“aCD70-AS269”) in a U266 multiple myeloma model (see Example 13).

FIG. 11 shows a graphical illustration of survival curves in a U266 multiple myeloma mouse model after a single injection of Anti-CD70-AS269 or unconjugated mAb (see Example 13).

FIG. 12 shows a graphical illustration of CD27 signaling inhibition by anti-CD70-AS269 ADC (“aCD70-AS269”) in a CD27 Reporter/Caki-1 coculture assay (see Example 14).

FIG. 13 shows anti-CD70-AS269 ADC affinity for human, cynomolgus, rat and mouse CD70 as measured by surface plasmon resonance.

FIG. 14 shows a graphical illustration of the anti-CD70-AS269 ADC toxicokinetic and pharmacokinetic concentration-time curves, showing a clear therapeutic index (see Example 17).

FIG. 15 shows tumor volume measurements in a 786-OS3/PBMC model after days of treatment with ARX305, pembrolizumab or combined ARX305 and pembrolizumab.

FIG. 16 shows body mass measurements in the 786-OS3/PBMC model after days of treatment with ARX305, pembrolizumab or combined ARX305 and pembrolizumab.

### DETAILED DESCRIPTION OF THE INVENTION

Before describing the present invention in detail, it is to be understood that this invention is not limited to particular methodologies, or compositions, or biological systems, which can, of course, vary. It is also to be understood that the terminology used herein is for the purpose of describing particular embodiments only and is not intended to be limiting.

While various embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless otherwise defined herein or below in the remainder of the specification, all technical and scientific terms used herein have the same meaning as commonly understood by those of ordinary skill in the art to which the invention belongs. As used herein and in the appended claims, the singular forms “a,” “an,” and “the” include plural reference unless the context clearly indicates otherwise.

Unless defined otherwise, all technical and scientific terms used herein have the same meaning as commonly understood to one of ordinary skill in the art to which the inventions described herein belong. Various methods, materials, and the like, similar or equivalent to those described herein can be used in the practice or testing of the inventions described herein.

The term “alkyl” by itself or as part of another molecule as used herein means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another molecule as used herein means a divalent radical derived from an alkane, as exemplified, by (—CH₂—)ₙ, wherein n may be 1 to about 24. By way of example only, such groups include, but are not limited to, groups having 10 or fewer carbon atoms such as the structures —CH₂CH₂— and —CH₂CH₂CH₂CH₂—. A “lower alkyl” or “lower alkylene” is a shorter chain alkyl or alkylene group, generally having eight or fewer carbon atoms. The term “alkylene,” unless otherwise noted, is also meant to include those groups described herein as “heteroalkylene.”

The term “amino acid” as used herein refers to naturally occurring and non-natural amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to compounds that have the same basic chemical structure as a naturally occurring amino acid, by way of example only, an α-carbon that is bound to a hydrogen, a carboxyl group, an amino group, and an R group. Such analogs may have modified R groups (by way of example, norleucine) or may have modified peptide backbones while still retaining the same basic chemical structure as a naturally occurring amino acid. Non-limiting examples of amino acid analogs include homoserine, norleucine, methionine sulfoxide, methionine methyl sulfonium.

Amino acids may be referred to herein by either their name, their commonly known three letter symbols or by the one-letter symbols recommended by the IUPAC-IUB Biochemical Nomenclature Commission. Additionally, nucleotides, may be referred to by their commonly accepted single-letter codes.

The term “antibody” as used herein refers to a protein consisting of one or more polypeptides substantially encoded by all or part of the antibody genes. The immunoglobulin genes include, but are not limited to, the kappa, lambda, alpha, gamma (IgG1, IgG2, IgG3, and IgG4), delta, epsilon and mu constant region genes, as well as the myriad immunoglobulin variable region genes. Antibody herein is meant to include full-length antibodies and antibody fragments, and include antibodies that exist naturally in any organism or are engineered (e.g. are variants). Thus, unless specifically noted otherwise, statements and claims that use the term “antibody” or “antibodies” specifically includes “antibody fragment” and “antibody fragments.” The term “antibody” encompasses intact antibody, monoclonal or polyclonal antibodies. The term “antibody” also encompasses, multispecific antibodies such as bispecific antibodies. Human antibodies are usually made of two light chains and two heavy chains each comprising variable regions and constant regions. The light chain variable region comprises 3 CDRs, identified herein as CDRL1, CDRL2 and CDRL3 flanked by framework regions. The heavy chain variable region comprises 3 CDRs, identified herein as CDRH1, CDRH2 and CDRH3 flanked by framework regions.

Anti-CD70 antibodies known in the art are suitable for use with this invention. Any known heavy chain sequence can be combined with light chain sequences and in some embodiments of the present invention there is non-naturally encoded amino acid in the constant region of the antibodies. Accordingly, in one aspect, this disclosure provides an isolated monoclonal antibody or antigen binding portion thereof comprising: (a) a heavy chain comprising an amino acid sequence selected from the group consisting of SEQ ID NOs: 1, 3, 4 and 5; (b) a light chain comprising an amino acid sequence selected from the group consisting of SEQ ID NOs: 2, 6, 7, 8 and 9; and (c) a non-naturally encoded amino acid in a heavy chain, a light chain, or both; wherein the antibody specifically binds to CD70. In some embodiments, the non-naturally encoded amino acid is para-acetyl-L-phenylalanine (pAF). In another aspect, this disclosure provides an isolated monoclonal antibody or antigen binding portion thereof comprising: (a) a heavy chain consisting of an amino acid sequence selected from the group consisting of SEQ ID NOs: 1, 3, 4 and 5; (b) a light chain consisting of an amino acid sequence selected from the group consisting of SEQ ID NOs: 2, 6, 7, 8 and 9; and (c) a non-naturally encoded amino acid in a heavy chain, a light chain, or both; wherein the antibody specifically binds to CD70. In some aspects, the non-naturally encoded amino acid is para-acetyl-L-phenylalanine (pAF). In another aspect, this disclosure provides an isolated monoclonal antibody or antigen binding portion thereof comprising: (a) a heavy chain consisting of an amino acid sequence of SEQ ID NO: 3; (b) a light chain consisting of an amino acid sequence of SEQ ID NOs: 2; and (c) a non-naturally encoded amino acid in a heavy chain, a light chain, or both; wherein the antibody specifically binds to CD70. In some aspects, the non-naturally encoded amino acid is para-acetyl-L-phenylalanine (pAF). Anti-CD70 (aCD70) antibodies known in the art are suitable for use in the present invention.

The term “antigen-binding fragment” as used herein refers to one or more fragments of an antibody that retain the ability to bind to an antigen. It has been shown that the antigen-binding function of an antibody can be performed by fragments of an intact antibody. Examples of binding fragments encompassed within the term “antigen-binding fragment” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR), e.g., V_{H} CDR3 comprising or not additional sequence (linker, framework region(s) etc.) and (v) a combination of two to six isolated CDRs comprising or not additional sequence (linker, framework region(s) etc.). Furthermore, although the two domains of the Fv fragment, V_{L} and V_{H}, are coded for by separate genes, they can be joined, using recombinant methods, by a synthetic linker that enables them to be made as a single polypeptide chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. Furthermore, the antigen-binding fragments include binding-domain immunoglobulin fusion proteins comprising (i) a binding domain polypeptide (such as a heavy chain variable region, a light chain variable region, or a heavy chain variable region fused to a light chain variable region via a linker peptide) that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The hinge region may be modified by replacing one or more cysteine residues with serine residues to prevent dimerization. Such binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

A typical antigen binding site is comprised of the variable regions formed by the pairing of a light chain immunoglobulin and a heavy chain immunoglobulin. The structure of the antibody variable regions is very consistent and exhibits very similar structures. These variable regions are typically comprised of relatively homologous framework regions (FR) interspaced with three hypervariable regions termed Complementarity Determining Regions (CDRs). The overall binding activity of the antigen binding fragment is often dictated by the sequence of the CDRs. The FRs often play a role in the proper positioning and alignment in three dimensions of the CDRs for optimal antigen binding.

In fact, because CDR sequences are responsible for most antibody-antigen interactions, it is possible to express recombinant antibodies that shows the properties of specific naturally occurring antibodies by constructing expression vectors that include CDR sequences from the specific naturally occurring antibody grafted onto framework sequences from a different antibody with different properties (see, e.g., Riechmann, L. et al., 1998, Nature 332:323-327; Jones, P. et al., 1986, Nature 321:522-525; and Queen, C. et al., 1989, Proc. Natl. Acad. See. U.S.A. 86:10029-10033). Such framework sequences can be obtained from public DNA databases that include germline antibody gene sequences. These germline sequences may differ from mature antibody gene sequences because they may not include completely assembled variable genes, which are formed by V(D)J joining during B cell maturation. Germline gene sequences may also differ from the sequences of a high affinity secondary repertoire antibody which contains mutations throughout the variable gene but typically clustered in the CDRs. For example, somatic mutations are relatively infrequent in the amino terminal portion of framework region 1 and in the carboxy-terminal portion of framework region 4. Furthermore, many somatic mutations do not significantly alter the binding properties of the antibody. For this reason, it is not necessary to obtain the entire DNA sequence of a particular antibody in order to recreate an intact recombinant antibody having binding properties similar to those of the original antibody. Partial heavy and light chain sequence spanning the CDR regions is typically sufficient for this purpose. The partial sequence is used to determine which germline variable and joining gene segments contributed to the recombined antibody variable genes. The germline sequence is then used to fill in missing portions of the variable regions. Heavy and light chain leader sequences are cleaved during protein maturation and do not contribute to the properties of the final antibody. To add missing sequences, cloned cDNA sequences can be combined with synthetic oligonucleotides by ligation or PCR amplification. Alternatively, the entire variable region can be synthesized to create an entirely synthetic variable region clone. This process has certain advantages such as elimination or inclusion of particular restriction sites, or optimization of particular codons.

Of course, the totality or portions of the framework region of the antibody described herein may be used in conjunction with the CDRs to optimize the affinity, specificity or any other desired properties of the antibody.

The term “antibody fragment” as used herein refers to any form of an antibody other than the full-length form. Antibody fragments herein include antibodies that are smaller components that exist within full-length antibodies, and antibodies that have been engineered. Antibody fragments include but are not limited to Fv, Fc, Fab, and (Fab′)2, single chain Fv (scFv), diabodies, triabodies, tetrabodies, bifunctional hybrid antibodies, CDR1, CDR2, CDR3, combinations of CDR's, variable regions, framework regions, constant regions, heavy chains, light chains, and variable regions, and alternative scaffold non-antibody molecules, bispecific antibodies, and the like (Maynard & Georgiou, 2000, Annu. Rev. Biomed. Eng. 2:339-76; Hudson, 1998, Curr. Opin. Biotechnol. 9:395-402). Another functional substructure is a single chain Fv (scFv), comprised of the variable regions of the immunoglobulin heavy and light chain, covalently connected by a peptide linker (S-z Hu et al., 1996, Cancer Research, 56, 3055-3061). These small (Mr 25,000) proteins generally retain specificity and affinity for antigen in a single polypeptide and can provide a convenient building block for larger, antigen-specific molecules.

The term “antibody-drug conjugate” or “ADC” as used herein refers to an antibody molecule, or fragment thereof, that is covalently bonded to one or more biologically active molecule(s). The biologically active molecule may be conjugated to the antibody through a linker, polymer, or other covalent bond.

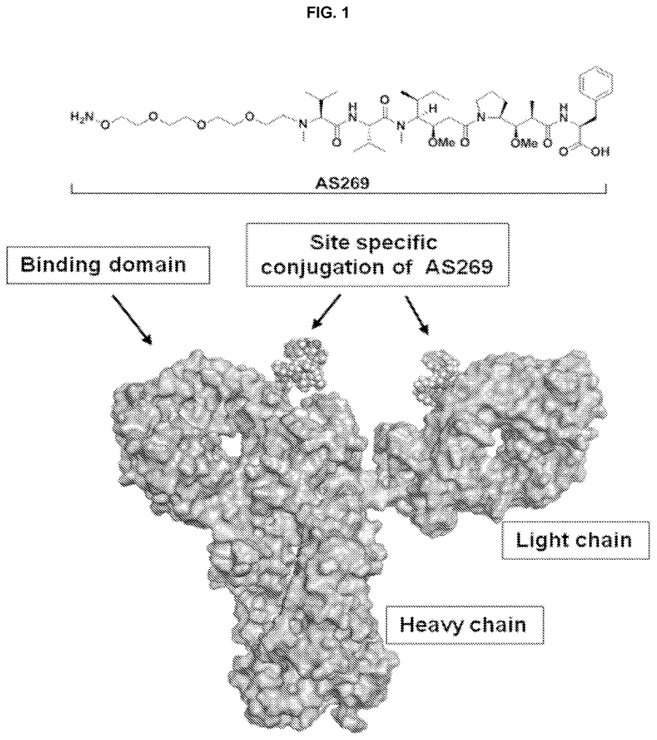

As used herein, the terms “ARX305”, “anti-CD70-AS269”, or “anti-CD70-AS269 ADC”, refer to an ADC containing (i) humanized monoclonal antibody (mAb; subclass IgG1) with specificity for human CD70; and (2) drug-linker AS269; (see FIG. 1). The humanized mAb with specificity for human CD70 contains two heavy chains and two light chains; the amino acid sequence of each heavy chain is SEQ ID NO: 3, which contains one non-natural amino acid para-acetyl-L-phenylalanine (pAF), which is genetically encoded and biosynthetically incorporated at amino acid position 114 (Kabat numbering) of each heavy chain; and the amino acid sequence of each light chain is SEQ ID NO: 2. AS269 is site-specifically conjugated via an oxime bond to the pAF incorporated at amino acid position 114 (Kabat numbering) of each anti-CD70 mAb heavy chain (one drug-linker per heavy chain), thereby the ARX305 drug-to-antibody ratio (DAR) is about 2. Compositions containing the ARX305 ADC (e.g., ARX305 pharmaceutical formulations including reconstituted solutions) can have a DAR of about 1.9 (see Example 19).

The term “bifunctional polymer” (also referred to as a “bifunctional linker”) as used herein refers to a polymer comprising two functional groups that are capable of reacting specifically with other moieties to form covalent or non-covalent linkages. Such moieties may include, but are not limited to, the side groups on natural or non-natural amino acids or peptides which contain such natural or non-natural amino acids. The other moieties that may be linked to the bifunctional linker or bifunctional polymer may be the same or different moieties. By way of example only, a bifunctional linker may have a functional group reactive with a group on a first peptide, and another functional group which is reactive with a group on a second peptide, whereby forming a conjugate that includes the first peptide, the bifunctional linker and the second peptide. Many procedures and linker molecules for attachment of various compounds to peptides are known. See, e.g., European Patent Application No. 188,256; U.S. Pat. Nos. 4,671,958, 4,659,839, 4,414,148, 4,699,784; 4,680,338; and 4,569,789 which are incorporated by reference herein in their entirety. A “multi-functional polymer” also referred to as a “multi-functional linker”, refers to a polymer comprising two or more functional groups that are capable of reacting with other moieties. Such moieties may include, but are not limited to, the side groups on natural or non-natural amino acids or peptides which contain such natural or non-natural amino acids. (including but not limited to, amino acid side groups) to form covalent or non-covalent linkages. A bi-functional polymer or multi-functional polymer may be any desired length or molecular weight, and may be selected to provide a particular desired spacing or conformation between one or more molecules linked to a compound and molecules it binds to or the compound.

The term “bioavailability” as used herein refers to the rate and extent to which a substance or its active moiety is delivered from a pharmaceutical dosage form and becomes available at the site of action or in the general circulation. Increases in bioavailability refers to increasing the rate and extent a substance or its active moiety is delivered from a pharmaceutical dosage form and becomes available at the site of action or in the general circulation. By way of example, an increase in bioavailability may be indicated as an increase in concentration of the substance or its active moiety in the blood when compared to other substances or active moieties. A non-limiting example of a method to evaluate increases in bioavailability is given in examples 21-25. This method may be used for evaluating the bioavailability of any polypeptide.

The term “biologically active molecule”, “biologically active moiety” or “biologically active agent” as used herein means any substance which can affect any physical or biochemical properties of a biological system, pathway, molecule, or interaction relating to an organism, including but not limited to, viruses, bacteria, bacteriophage, transposon, prion, insects, fungi, plants, animals, and humans. In particular, as used herein, biologically active molecules include but are not limited to any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes, small molecule drugs, hard drugs, soft drugs, prodrugs, carbohydrates, inorganic atoms or molecules, dyes, lipids, nucleosides, radionuclides, oligonucleotides, toxins, cells, viruses, liposomes, microparticles and micelles. Classes of biologically active agents that are suitable for use with the methods and compositions described herein include, but are not limited to, drugs, prodrugs, radionuclides, imaging agents, polymers, antibiotics, fungicides, anti-viral agents, anti-inflammatory agents, anti-tumor agents, cardiovascular agents, anti-anxiety agents, hormones, growth factors, steroidal agents, microbially derived toxins, and the like.

The term “modulating biological activity” as used herein refers to increasing or decreasing the reactivity of a polypeptide, altering the selectivity of the polypeptide, enhancing or decreasing the substrate selectivity of the polypeptide. Analysis of modified biological activity can be performed by comparing the biological activity of the non-natural polypeptide to that of the natural polypeptide.

The term “biosynthetically” as used herein refers to any method utilizing a translation system (cellular or non-cellular), including use of at least one of the following components: a polynucleotide, a codon, a tRNA, and a ribosome. In a non-limiting example, non-natural amino acids may be “biosynthetically incorporated” into non-natural amino acid polypeptides using the methods and techniques described in U.S. Pat. No. 7,083,970 and in US 2021/0017527, the entire contents of each of which are hereby incorporated by reference in their entirety.

The term “carbonyl” as used herein refers to the moiety —C(O)—. Non-limiting examples of groups containing carbonyl include a ketone, aldehyde, carboxylic acid and ester. In addition, a carbonyl can be part of linear, branched or cyclic molecules.

The term “chemically cleavable group” (also referred to as “chemically labile”) as used herein refers to a group which breaks or cleaves upon exposure to acid, base, oxidizing agents, reducing agents, chemical inititiators, or radical initiators.

The term “chemiluminescent group” as used herein refers to a group which emits light as a result of a chemical reaction without the addition of heat. By way of example only, luminol (5-amino-2,3-dihydro-1,4-phthalazinedione) reacts with oxidants like hydrogen peroxide (H₂O₂) in the presence of a base and a metal catalyst to produce an excited state product (3-aminophthalate, 3-APA).

The term “chromophore” as used herein refers to a molecule which absorbs light of visible wavelengths, UV wavelengths or IR wavelengths.

The term “comparison window” as used herein refers a segment of any one of contiguous positions used to compare a sequence to a reference sequence of the same number of contiguous positions after the two sequences are optimally aligned. Such contiguous positions include, but are not limited to a group consisting of from about 20 to about 600 sequential units, including about 50 to about 200 sequential units, and about 100 to about 150 sequential units. By way of example only, such sequences include polypeptides and polypeptides containing non-natural amino acids, with the sequential units include, but are not limited to natural and non-natural amino acids. In addition, by way of example only, such sequences include polynucleotides with nucleotides being the corresponding sequential units. Methods of alignment of sequences for comparison are well-known in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

By way of example, an algorithm which may be used to determine percent sequence identity and sequence similarity are the BLAST and BLAST 2.0 algorithms, which are described in Altschul et al. (1997) Nuc. Acids Res. 25:3389-3402, and Altschul et al. (1990) J. Mol. Biol. 215:403-410, respectively. Software for performing BLAST analyses is publicly available through the National Center for Biotechnology Information. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

The term “conservatively modified variants” as used herein applies to both natural and non-natural amino acid and natural and non-natural nucleic acid sequences, and combinations thereof. With respect to particular nucleic acid sequences, “conservatively modified variants” refers to those natural and non-natural nucleic acids which encode identical or essentially identical natural and non-natural amino acid sequences, or where the natural and non-natural nucleic acid does not encode a natural and non-natural amino acid sequence, to essentially identical sequences. By way of example, because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Thus by way of example every natural or non-natural nucleic acid sequence herein which encodes a natural or non-natural polypeptide also describes every possible silent variation of the natural or non-natural nucleic acid. One of ordinary skill in the art will recognize that each codon in a natural or non-natural nucleic acid (except AUG, which is ordinarily the only codon for methionine, and TGG, which is ordinarily the only codon for tryptophan) can be modified to yield a functionally identical molecule. Accordingly, each silent variation of a natural and non-natural nucleic acid which encodes a natural and non-natural polypeptide is implicit in each described sequence.

As to amino acid sequences, individual substitutions, deletions or additions to a nucleic acid, peptide, polypeptide, or protein sequence which alters, adds or deletes a single natural and non-natural amino acid or a small percentage of natural and non-natural amino acids in the encoded sequence is a “conservatively modified variant” where the alteration results in the deletion of an amino acid, addition of an amino acid, or substitution of a natural and non-natural amino acid with a chemically similar amino acid. Conservative substitution tables providing functionally similar natural amino acids are well known in the art. Such conservatively modified variants are in addition to and do not exclude polymorphic variants, interspecies homologs, and alleles of the methods and compositions described herein.

Conservative substitution tables providing functionally similar amino acids are known to those of ordinary skill in the art. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M); (see, e.g., Creighton, Proteins: Structures and Molecular Properties, W H Freeman & Co.; 2^{nd} edition, December 1993).

The term “cytotoxic” as used herein refers to a compound which harms cells.

The term “diamine” as used herein refers to groups/molecules comprising at least two amine functional groups, including, but not limited to, a hydrazine group, an amidine group, an imine group, a 1,1-diamine group, a 1,2-diamine group, a 1,3-diamine group, and a 1,4-diamine group. In addition, such groups may be part of linear, branched, or cyclic molecules.

The term “drug” as used herein refers to any substance used in the prevention, diagnosis, alleviation, treatment, or cure of a disease or condition.

The term “effective amount” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. By way of example, an agent or a compound being administered includes, but is not limited to, a natural amino acid polypeptide, non-natural amino acid polypeptide, modified natural amino acid polypeptide, or modified non-amino acid polypeptide. Compositions containing such natural amino acid polypeptides, non-natural amino acid polypeptides, modified natural amino acid polypeptides, or modified non-natural amino acid polypeptides can be administered for prophylactic, enhancing, and/or therapeutic treatments. An appropriate “effective” amount in any individual case may be determined using techniques, such as a dose escalation study.

The terms “enhance” or “enhancing” as used herein means to increase or prolong either in potency or duration a desired effect. By way of example, “enhancing” the effect of therapeutic agents refers to the ability to increase or prolong, either in potency or duration, the effect of therapeutic agents on during treatment of a disease, disorder or condition. An “enhancing-effective amount,” as used herein, refers to an amount adequate to enhance the effect of a therapeutic agent in the treatment of a disease, disorder or condition. When used in a patient, amounts effective for this use will depend on the severity and course of the disease, disorder or condition, previous therapy, the patient's health status and response to the drugs, and the judgment of the treating physician.

The term “eukaryote” as used herein refers to organisms belonging to the phylogenetic domain Eucarya, including but not limited to animals (including but not limited to, mammals, insects, reptiles, birds, etc.), ciliates, plants (including but not limited to, monocots, dicots, and algae), fungi, yeasts, flagellates, microsporidia, and protists.

The terms “functional group”, “active moiety”, “activating group”, “leaving group”, “reactive site”, “chemically reactive group” and “chemically reactive moiety” as used herein refer to portions or units of a molecule at which chemical reactions occur. The terms are somewhat synonymous in the chemical arts and are used herein to indicate the portions of molecules that perform some function or activity and are reactive with other molecules.

The term “identical” as used herein refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using comparison algorithms or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence. By way of example only, two or more polypeptide sequences are identical when the amino acid residues are the same, while two or more polypeptide sequences are “substantially identical” if the amino acid residues are about 60% identical, about 65% identical, about 70% identical, about 75% identical, about 80% identical, about 85% identical, about 90% identical, or about 95% identical over a specified region. The identity can exist over a region that is at least about 75 to about 100 amino acids in length, over a region that is about 50 amino acids in length, or, where not specified, across the entire sequence of a polypeptide sequence. In addition, by way of example only, two or more polynucleotide sequences are identical when the nucleic acid residues are the same, while two or more polynucleotide sequences are “substantially identical” if the nucleic acid residues are about 60% identical, about 65% identical, about 70% identical, about 75% identical, about 80% identical, about 85% identical, about 90% identical, or about 95% identical over a specified region. The identity can exist over a region that is at least about 75 to about 100 nucleic acids in length, over a region that is about 50 nucleic acids in length, or, where not specified, across the entire sequence of a polynucleotide sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term “immunogenicity” as used herein refers to an antibody response to administration of a therapeutic drug. The immunogenicity toward therapeutic non-natural amino acid polypeptides can be obtained using quantitative and qualitative assays for detection of anti-non-natural amino acid polypeptides antibodies in biological fluids. Such assays include, but are not limited to, Radioimmunoassay (RIA), Enzyme-linked immunosorbent assay (ELISA), luminescent immunoassay (LIA), and fluorescent immunoassay (FIA). Analysis of immunogenicity toward therapeutic non-natural amino acid polypeptides involves comparing the antibody response upon administration of therapeutic non-natural amino acid polypeptides to the antibody response upon administration of therapeutic natural amino acid polypeptides.

The term “isolated” as used herein refers to separating and removing a component of interest from components not of interest. Isolated substances can be in either a dry or semi-dry state, or in solution, including but not limited to an aqueous solution. The isolated component can be in a homogeneous state, or the isolated component can be a part of a pharmaceutical composition that comprises additional pharmaceutically acceptable carriers and/or excipients. Purity and homogeneity may be determined using analytical chemistry techniques including, but not limited to, polyacrylamide gel electrophoresis or high-performance liquid chromatography. In addition, when a component of interest is isolated and is the predominant species present in a preparation, the component is described herein as substantially purified. The term “purified,” as used herein, may refer to a component of interest which is at least 85% pure, at least 90% pure, at least 95% pure, at least 99% or greater pure. By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Also, by way of example, a gene is isolated when separated from open reading frames which flank the gene and encode a protein other than the gene of interest.

The term “label” as used herein, refers to a substance which is incorporated into a compound and is readily detected, whereby its physical distribution may be detected and/or monitored.

The term “linkage” or “adduct moiety” as used herein refers to a bond or chemical moiety formed from a chemical reaction between the functional group of one group, such as a linker of the present disclosure, and another molecule. Such bonds may include, but are not limited to, covalent linkages and non-covalent bonds, while such chemical moieties may include, but are not limited to, esters, carbonates, imines, phosphate esters, hydrazones, acetals, orthoesters, peptide linkages, oximes and oligonucleotide linkages. Hydrolytically stable linkages mean that the linkages are substantially stable in water and do not react with water at useful pH values, including but not limited to, under physiological conditions for an extended period of time, perhaps even indefinitely. Hydrolytically unstable or degradable linkages mean that the linkages are degradable in water or in aqueous solutions, including for example, blood. Enzymatically unstable or degradable linkages mean that the linkage can be degraded by one or more enzymes. By way of example only, PEG and related polymers may include degradable linkages in the polymer backbone or in the linker group between the polymer backbone and one or more of the terminal functional groups of the polymer molecule. Such degradable linkages include but are not limited to ester linkages formed by the reaction of PEG carboxylic acids or activated PEG carboxylic acids with alcohol groups on a biologically active agent, wherein such ester groups generally hydrolyze under physiological conditions to release the biologically active agent. Other hydrolytically degradable linkages include but are not limited to carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

The term “linker” as used herein refers to any multivalent group that connects, or is capable of connecting, a first group to at least one other group. Typically, a linker is a bivalent or a trivalent organic moiety that connects a drug or payload (first group) to a biologically active agent (second group), e.g., via a linkage or adduct moiety, or that connects a drug or payload (first group) to a reactive moiety (second group), wherein the reactive moiety is capable of reacting with a biologically active agent. Linkers can be susceptible to cleavage (cleavable linkers), such as, acid-induced cleavage, photo-induced cleavage, peptidase-induced cleavage, esterase-induced cleavage, and disulfide bond cleavage, and so on, at conditions under which the drug or payload and the at least one other group remains active. Alternatively, linkers can be substantially resistant to cleavage (e.g., stable linker or non-cleavable linker).

In some embodiments, the linker is a bivalent or trivalent group comprising, or consisting of, at least one moiety, wherein each at least one moiety is independently selected from the group consisting of a bond, unsubstituted alkylene, substituted alkylene, -(alkylene-O)ₙ—, optionally substituted arylene, —O—, —C(O)—, —C(S)—, —N(R_{w})—, —S(O)₀₋₂—, methine (—CH)—, an amino acid, a peptide, a disulfide (—S—S—) and a phosphate-containing moiety; and combinations thereof, wherein: each R_{w} is independently H, C₁-C₈ alkyl or a bond; and each phosphate-containing moiety is independently selected from the group consisting of a phosphate ester, a pyrophosphate ester, a triphosphate ester, a tetraphosphate ester, a phosphonate, a diphosphonate, a phosporamidate, a pyrophosporamidate, a triphosphoramidate, a tetraphosphoramidate, a phosphorthioate and a diphosphorthioate. Unless expressly indicated otherwise, no orientation of the linker is implied by the direction in which the formula of the linker group is written. By way of example, the formula —C(O)CH₂CH₂— represents both —C(O)CH₂CH₂— and —CH₂CH₂C(O)—. In another example, the formula —C(O)CH₂CH₂— represents both *—C(O)CH₂CH₂— and —C(O)CH₂CH₂—*, wherein * denotes a point of connection, for example, connection to a drug or payload. In some embodiments, when a selected moiety occurs two or more times in the same linker, the two or more occurrences are not adjacent. In some embodiments, a linker is not a bond.

In some embodiments, a linker is a bivalent group that connects a first group and a second group. In some other embodiments, the linker is a trivalent moiety that connects a first group, a second group and a third group.

In some embodiments, a linker connects at least a first group and a second group, wherein the first group is a drug or payload, and the second group is a biologically active polypeptide or protein. In some embodiments, the biologically active polypeptide or protein contains at least one non-naturally encoded amino acid. In some embodiments, the linker connects the drug or payload to a non-naturally encoded amino acid of the biologically active polypeptide or protein. In some embodiments, the biologically active polypeptide or protein is an antibody. Thus, the antibody connected to a drug or payload via a linker can be an antibody-drug conjugate (ADC), such as an ADC of the present disclosure.

In some other embodiments, a linker connects at least a first group and a second group, wherein the first group is a drug or payload, and the second group is a reactive moiety. In some embodiments, the second group is a reactive moiety that can react with a biologically active polypeptide or protein. In some embodiments, the biologically active polypeptide or protein contains at least one non-naturally encoded amino acid. Thus, in some embodiments, the reactive moiety can react with a non-naturally encoded amino acid of the biologically active polypeptide or protein. In some embodiments, the biologically active polypeptide or protein is an antibody.

In some embodiments, a first linker is connected to a second linker, and the combined linkers (a composite linker) connects at least a first group and a second group. A composite linker of the present disclosure can contain 2, 3, 4, 5, 6, 7, 8, 9, 10 or more linker groups. In a non-limiting example, a first, second and third linker group are joined together to provide a composite linker that can connect a first group (e.g., a drug or payload) to at least one other group, such as a reactive moiety and/or a biologically active polypeptide or protein (e.g., an antibody. In some embodiments, the biologically active polypeptide or protein (e.g., antibody) contains a non-naturally encoded amino acid.

In some embodiments, a linker is linear. In other embodiments, a linker is branched.

The terms “medium” or “media” as used herein refer to any culture medium used to grow and harvest cells and/or products expressed and/or secreted by such cells. Such “medium” or “media” include, but are not limited to, solution, solid, semi-solid, or rigid supports that may support or contain any host cell, including, by way of example, bacterial host cells, yeast host cells, insect host cells, plant host cells, eukaryotic host cells, mammalian host cells, CHO cells, prokaryotic host cells, E. coli, or Pseudomonas host cells, and cell contents. Such “medium” or “media” includes, but is not limited to, medium or media in which the host cell has been grown into which a polypeptide has been secreted, including medium either before or after a proliferation step. Such “medium” or “media” also includes, but is not limited to, buffers or reagents that contain host cell lysates, by way of example a polypeptide produced intracellularly, and the host cells are lysed or disrupted to release the polypeptide.

The term “metabolite” as used herein refers to a derivative of a compound, by way of example natural amino acid polypeptide, a non-natural amino acid polypeptide, a modified natural amino acid polypeptide, or a modified non-natural amino acid polypeptide, that is formed when the compound, by way of example natural amino acid polypeptide, non-natural amino acid polypeptide, modified natural amino acid polypeptide, or modified non-natural amino acid polypeptide, is metabolized. The term “pharmaceutically active metabolite” or “active metabolite” refers to a biologically active derivative of a compound, by way of example natural amino acid polypeptide, a non-natural amino acid polypeptide, a modified natural amino acid polypeptide, or a modified non-natural amino acid polypeptide, that is formed when such a compound, by way of example a natural amino acid polypeptide, non-natural amino acid polypeptide, modified natural amino acid polypeptide, or modified non-natural amino acid polypeptide, is metabolized.

The term “metabolized” as used herein refers to the sum of the processes by which a particular substance is changed by an organism. Such processes include, but are not limited to, hydrolysis reactions and reactions catalyzed by enzymes. Further information on metabolism may be obtained from The Pharmacological Basis of Therapeutics, 9^{th} Edition, McGraw-Hill (1996). By way of example only, metabolites of natural amino acid polypeptides, non-natural amino acid polypeptides, modified natural amino acid polypeptides, or modified non-natural amino acid polypeptides may be identified either by administration of the natural amino acid polypeptides, non-natural amino acid polypeptides, modified natural amino acid polypeptides, or modified non-natural amino acid polypeptides to a host and analysis of tissue samples from the host, or by incubation of natural amino acid polypeptides, non-natural amino acid polypeptides, modified natural amino acid polypeptides, or modified non-natural amino acid polypeptides with hepatic cells in vitro and analysis of the resulting compounds.

The term “metal chelator” as used herein refers to a molecule which forms a metal complex with metal ions. By way of example, such molecules may form two or more coordination bonds with a central metal ion and may form ring structures.

The term “modified” as used herein refers to the presence of a change to a natural amino acid, a non-natural amino acid, a natural amino acid polypeptide or a non-natural amino acid polypeptide. Such changes, or modifications, may be obtained by post synthesis modifications of natural amino acids, non-natural amino acids, natural amino acid polypeptides or non-natural amino acid polypeptides, or by co-translational, or by post-translational modification of natural amino acids, non-natural amino acids, natural amino acid polypeptides or non-natural amino acid polypeptides. The form “modified or unmodified” means that the natural amino acid, non-natural amino acid, natural amino acid polypeptide or non-natural amino acid polypeptide being discussed are optionally modified, that is, he natural amino acid, non-natural amino acid, natural amino acid polypeptide or non-natural amino acid polypeptide under discussion can be modified or unmodified.

The term “modulated serum half-life” as used herein refers to positive or negative changes in the circulating half-life of a modified biologically active molecule relative to its non-modified form. By way of example, the modified biologically active molecules include, but are not limited to, natural amino acid, non-natural amino acid, natural amino acid polypeptide or non-natural amino acid polypeptide. By way of example, serum half-life is measured by taking blood samples at various time points after administration of the biologically active molecule or modified biologically active molecule and determining the concentration of that molecule in each sample. Correlation of the serum concentration with time allows calculation of the serum half-life. By way of example, modulated serum half-life may be an increase in serum half-life, which may enable an improved dosing regimens or avoid toxic effects. Such increases in serum may be at least about two-fold, at least about three-fold, at least about five-fold, or at least about ten-fold. Methods for evaluating serum half-life are known in the art and may be used for evaluating the serum half-life of antibodies and antibody drug conjugates of the present invention.

The term “modulated therapeutic half-life” as used herein refers to positive or negative change in the half-life of the therapeutically effective amount of a modified biologically active molecule, relative to its non-modified form. By way of example, the modified biologically active molecules include, but are not limited to, natural amino acid, non-natural amino acid, natural amino acid polypeptide or non-natural amino acid polypeptide. By way of example, therapeutic half-life is measured by measuring pharmacokinetic and/or pharmacodynamic properties of the molecule at various time points after administration. Increased therapeutic half-life may enable a particular beneficial dosing regimen, a particular beneficial total dose, or avoids an undesired effect. By way of example, the increased therapeutic half-life may result from increased potency, increased or decreased binding of the modified molecule to its target, an increase or decrease in another parameter or mechanism of action of the non-modified molecule, or an increased or decreased breakdown of the molecules by enzymes such as, by way of example only, proteases. Methods for evaluating therapeutic half-life are known in the art and may be used for evaluating the therapeutic half-life of antibodies and antibody drug conjugates of the present invention.

The term “near-stoichiometric” as used herein refers to the ratio of the moles of compounds participating in a chemical reaction being about 0.75 to about 1.5.

The term “non-eukaryote” as used herein refers to organisms that are not eukaryotic. By way of example, a non-eukaryotic organism may belong to the Eubacteria, (which includes but is not limited to, Escherichia coli, Thermus thermophilus, or Bacillus stearothermophilus, Pseudomonas fluorescens, Pseudomonas aeruginosa, Pseudomonas putida), phylogenetic domain, or the Archaea, which includes, but is not limited to, Methanococcus jannaschii, Methanobacterium thermoautotrophicum, Archaeoglobus fulgidus, Pyrococcus furiosus, Pyrococcus horikoshii, Aeuropyrum pernix, or Halobacterium such as Haloferax volcanii and Halobacterium species NRC-1, or phylogenetic domain.

The term “non-natural amino acid” as used herein refers to an amino acid that is not one of the 20 common amino acids or pyrolysine or selenocysteine. Other terms that may be used synonymously with the term “non-natural amino acid” is “non-naturally encoded amino acid,” “unnatural amino acid,” “non-naturally-occurring amino acid,” and variously hyphenated and non-hyphenated versions thereof. The term “non-natural amino acid” includes, but is not limited to, amino acids which occur naturally by modification of a naturally encoded amino acid (including but not limited to, the 20 common amino acids or pyrrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine. Additionally, the term “non-natural amino acid” includes, but is not limited to, amino acids which do not occur naturally and may be obtained synthetically or may be obtained by modification of non-natural amino acids.

The term “nucleic acid” as used herein refers to deoxyribonucleotides, deoxyribonucleosides, ribonucleosides or ribonucleotides and polymers thereof in either single- or double-stranded form. By way of example only, such nucleic acids and nucleic acid polymers include, but are not limited to, (i) analogues of natural nucleotides which have similar binding properties as a reference nucleic acid and are metabolized in a manner similar to naturally occurring nucleotides; (ii) oligonucleotide analogs including, but are not limited to, PNA (peptidonucleic acid), analogs of DNA used in antisense technology (phosphorothioates, phosphoroamidates, and the like); (iii) conservatively modified variants thereof (including but not limited to, degenerate codon substitutions) and complementary sequences and sequence explicitly indicated. By way of example, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “oxidizing agent” as used herein refers to a compound or material which can remove an electron from a compound being oxidized. By way of example oxidizing agents include, but are not limited to, oxidized glutathione, cystine, cystamine, oxidized dithiothreitol, oxidized erythreitol, and oxygen. A wide variety of oxidizing agents are suitable for use in the methods and compositions described herein.

The term “pharmaceutically acceptable” as used herein refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “photoaffinity label” as used herein refers to a label with a group, which, upon exposure to light, forms a linkage with a molecule for which the label has an affinity. By way of example only, such a linkage may be covalent or non-covalent.

The term “photocleavable group” as used herein refers to a group which breaks upon exposure to light.

The term “photocrosslinker” as used herein refers to a compound comprising two or more functional groups which, upon exposure to light, are reactive and form a covalent or non-covalent linkage with two or more monomeric or polymeric molecules.

The term “photoisomerizable moiety” as used herein, refers to a group wherein upon illumination with light changes from one isomeric form to another.

The term “polyalkylene glycol” as used herein refers to linear or branched polymeric polyether polyols. Such polyalkylene glycols, including, but are not limited to, polyethylene glycol, polypropylene glycol, polybutylene glycol, and derivatives thereof. Other exemplary embodiments are listed, for example, in commercial supplier catalogs, such as Shearwater Corporation's catalog “Polyethylene Glycol and Derivatives for Biomedical Applications” (2001).

The term “polymer” as used herein refers to a molecule composed of repeated subunits. Such molecules include, but are not limited to, polypeptides, polynucleotides, or polysaccharides or polyalkylene glycols.

The terms “polypeptide,” “peptide” and “protein” are used interchangeably herein, and as used herein refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-natural amino acid. Additionally, such “polypeptides,” “peptides” and “proteins” include amino acid chains of any length, including full length proteins, wherein the amino acid residues are linked by covalent peptide bonds.

The term “post-translationally modified” as used herein refers to any modification of a natural or non-natural amino acid which occurs after such an amino acid has been translationally incorporated into a polypeptide chain. Such modifications include, but are not limited to, co-translational in vivo modifications, co-translational in vitro modifications (such as in a cell-free translation system), post-translational in vivo modifications, and post-translational in vitro modifications.

The terms “prodrug” or “pharmaceutically acceptable prodrug” as used herein refers to an agent that is converted into the parent drug in vivo or in vitro, wherein which does not abrogate the biological activity or properties of the drug, and is relatively nontoxic, i.e., the material may be administered to an individual without causing undesirable biological effects or interacting in a deleterious manner with any of the components of the composition in which it is contained. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are converted into active drug within the body through enzymatic or non-enzymatic reactions. Prodrugs may provide improved physiochemical properties such as better solubility, enhanced delivery characteristics, such as specifically targeting a particular cell, tissue, organ or ligand, and improved therapeutic value of the drug. The benefits of such prodrugs include, but are not limited to, (i) ease of administration compared with the parent drug; (ii) the prodrug may be bioavailable by oral administration whereas the parent is not; and (iii) the prodrug may also have improved solubility in pharmaceutical compositions compared with the parent drug. A pro-drug includes a pharmacologically inactive, or reduced-activity, derivative of an active drug. Prodrugs may be designed to modulate the amount of a drug or biologically active molecule that reaches a desired site of action through the manipulation of the properties of a drug, such as physiochemical, biopharmaceutical, or pharmacokinetic properties. An example, without limitation, of a prodrug would be a non-natural amino acid polypeptide which is administered as an ester (the “prodrug”) to facilitate transmittal across a cell membrane where water solubility is detrimental to mobility, but which then is metabolically hydrolyzed to the carboxylic acid, the active entity, once inside the cell where water solubility is beneficial. Prodrugs may be designed as reversible drug derivatives, for use as modifiers to enhance drug transport to site-specific tissues.

The term “prophylactically effective amount” as used herein refers that amount of a composition containing at least one non-natural amino acid polypeptide or at least one modified non-natural amino acid polypeptide prophylactically applied to a patient which can relieve to some extent one or more of the symptoms of a disease, condition or disorder being treated. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

The term “protected” as used herein refers to the presence of a “protecting group” or moiety that prevents reaction of the chemically reactive functional group under certain reaction conditions. The protecting group may vary depending on the type of chemically reactive group being protected. By way of example only, (i) if the chemically reactive group is an amine or a hydrazide, the protecting group may be selected from tert-butyloxycarbonyl (t-Boc) and 9-fluorenylmethoxycarbonyl (Fmoc); (ii) if the chemically reactive group is a thiol, the protecting group may be orthopyridyldisulfide; and (iii) if the chemically reactive group is a carboxylic acid, such as butanoic or propionic acid, or a hydroxyl group, the protecting group may be benzyl or an alkyl group such as methyl, ethyl, or tert-butyl.

By way of example only, blocking/protecting groups may be selected from:

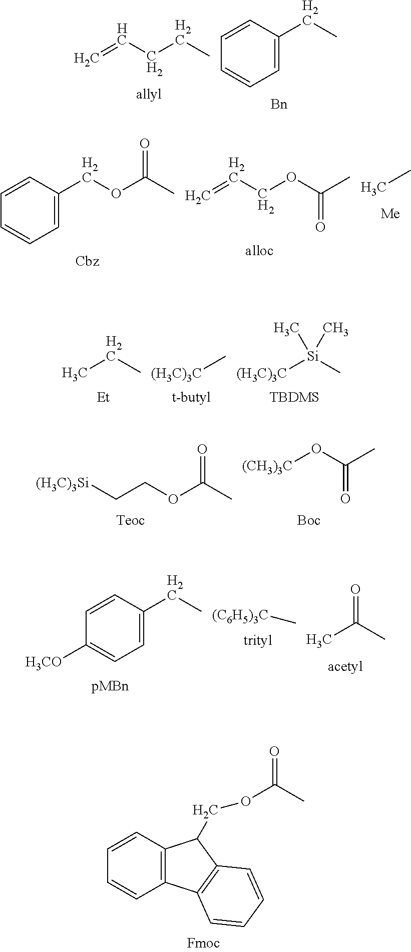

Additionally, protecting groups include, but are not limited to, including photolabile groups such as Nvoc and MeNvoc and other protecting groups known in the art. Other protecting groups are described in Greene and Wuts, Protective Groups in Organic Synthesis, 3^{rd} Ed., John Wiley & Sons, New York, NY, 1999, which is incorporated herein by reference in its entirety.

The term “reactive compound” as used herein refers to a compound which under appropriate conditions is reactive toward another atom, molecule or compound.

The term “recombinant host cell” (also referred to as “host cell”) as used herein refers to a cell which includes an exogenous polynucleotide, wherein the methods used to insert the exogenous polynucleotide into a cell include, but are not limited to, direct uptake, transduction, f-mating, or other methods known in the art to create recombinant host cells. By way of example only, such exogenous polynucleotide may be a nonintegrated vector, including but not limited to a plasmid, or may be integrated into the host genome.

The term “redox-active agent” as used herein refers to a molecule which oxidizes or reduces another molecule, whereby the redox active agent becomes reduced or oxidized. Examples of redox active agent include, but are not limited to, ferrocene, quinones, Ru^{2+/3+} complexes, Co^{2+/3+} complexes, and Os^{2+/3+} complexes.

The term “reducing agent” as used herein refers to a compound or material which can add an electron to a compound being reduced. By way of example reducing agents include, but are not limited to, dithiothreitol (DTT), 2-mercaptoethanol, dithioerythritol, cysteine, cysteamine (2-aminoethanethiol), and reduced glutathione. Such reducing agents may be used, by way of example only, to maintain sulfhydryl groups in the reduced state and to reduce intra- or intermolecular disulfide bonds.

The term “saccharide” as used herein refers to a series of carbohydrates including but not limited to sugars, monosaccharides, oligosaccharides, and polysaccharides.

The term “safety” or “safety profile” as used herein refers to side effects that might be related to administration of a drug relative to the number of times the drug has been administered. By way of example, a drug which has been administered many times and produced only mild or no side effects is said to have an excellent safety profile.

The phrase “selectively hybridizes to” or “specifically hybridizes to” as used herein refers to the binding, duplexing, or hybridizing of a molecule to a particular nucleotide sequence under stringent hybridization conditions when that sequence is present in a complex mixture including but not limited to, total cellular or library DNA or RNA.

The term “standard of care”, “best practice”, “standard medical care” or “standard therapy” as used herein refers to a treatment that is accepted by medical experts as proper treatment for a certain type of disease and that is widely used by healthcare professionals (see, e.g., https://www.cancer.gov/publications/dictionaries/cancer-terms/def/standard-of-care).

The term “stoichiometric” as used herein refers to the ratio of the moles of compounds participating in a chemical reaction being about 0.9 to about 1.1.

The term “stoichiometric-like” as used herein refers to a chemical reaction which becomes stoichiometric or near-stoichiometric upon changes in reaction conditions or in the presence of additives. Such changes in reaction conditions include, but are not limited to, an increase in temperature or change in pH. Such additives include, but are not limited to, accelerants.

The term “subject” as used herein refers to an animal which is the object of treatment, observation or experiment. By way of example only, a subject may be, but is not limited to, a mammal including, but not limited to, a human.

The term “substantially purified” as used herein refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater. By way of example only, a natural amino acid polypeptide or a non-natural amino acid polypeptide may be purified from a native cell, or host cell in the case of recombinantly produced natural amino acid polypeptides or non-natural amino acid polypeptides. By way of example a preparation of a natural amino acid polypeptide or a non-natural amino acid polypeptide may be “substantially purified” when the preparation contains less than about 30%, less than about 25%, less than about 20%, less than about 15%, less than about 10%, less than about 5%, less than about 4%, less than about 3%, less than about 2%, or less than about 1% (by dry weight) of contaminating material. By way of example when a natural amino acid polypeptide or a non-natural amino acid polypeptide is recombinantly produced by host cells, the natural amino acid polypeptide or non-natural amino acid polypeptide may be present at about 30%, about 25%, about 20%, about 15%, about 10%, about 5%, about 4%, about 3%, about 2%, or about 1% or less of the dry weight of the cells. By way of example when a natural amino acid polypeptide or a non-natural amino acid polypeptide is recombinantly produced by host cells, the natural amino acid polypeptide or non-natural amino acid polypeptide may be present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. By way of example, “substantially purified” natural amino acid polypeptides or non-natural amino acid polypeptides may have a purity level of about 30%, about 35%, about 40%, about 45%, about 50%, about 55%, about 60%, about 65%, about 70%, about 75%, about 80%, about 85%, about 90%, about 95%, about 99% or greater as determined by appropriate methods, including, but not limited to, SDS/PAGE analysis, RP-HPLC, SEC, and capillary electrophoresis.

The term “therapeutically effective amount” as used herein refers to the amount of a composition containing at least one non-natural amino acid polypeptide and/or at least one modified non-natural amino acid polypeptide or antibody-drug conjugate administered to a patient already suffering from a disease, condition or disorder, sufficient to cure or at least partially arrest, or relieve to some extent one or more of the symptoms of the disease, disorder or condition being treated. The effectiveness of such compositions depends on conditions including, but not limited to, the severity and course of the disease, disorder or condition, previous therapy, the patient's health status and response to the drugs, and the judgment of the treating physician. By way of example only, therapeutically effective amounts may be determined by routine experimentation, including but not limited to a dose escalation clinical trial.

The term “toxic moiety” or “toxic group” as used herein refers to a compound which can cause harm, disturbances, or death. Toxic moieties include, but are not limited to, NCA1, auristatin, DNA minor groove binding agent, DNA minor groove alkylating agent, enediyne, lexitropsin, duocarmycin, taxane, puromycin, dolastatin, maytansinoid, vinca alkaloid, AFP, monomethyl auristatin E (MMAF), monomethyl auristatin E (MMAE), AEB, AEVB, auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, chalicheamicin, maytansine, DM-1, netropsin, podophyllotoxin (e.g. etoposide, teniposide, etc.), baccatin and its derivatives, anti-tubulin agents, cryptophysin, combretastatin, auristatin E, vincristine, vinblastine, vindesine, vinorelbine, VP-16, camptothecin, epothilone A, epothilone B, nocodazole, colchicines, colcimid, estramustine, cemadotin, discodermolide, maytansine, eleutherobin, mechlorethamine, cyclophosphamide, melphalan, carmustine, lomustine, semustine, streptozocin, chlorozotocin, uracil mustard, chlormethine, ifosfamide, chlorambucil, pipobroman, triethylenemelamine, triethylenethiophosphoramine, busulfan, dacarbazine, and temozolomide, ytarabine, cytosine arabinoside, fluorouracil, floxuridine, 6-thioguanine, 6-mercaptopurine, pentostatin, 5-fluorouracil, methotrexate, 10-propargyl-5,8-dideazafolate, 5,8-dideazatetrahydrofolic acid, leucovorin, fludarabine phosphate, pentostatine, gemcitabine, Ara-C, paclitaxel, docetaxel, deoxycoformycin, mitomycin-C, L-asparaginase, azathioprine, brequinar, antibiotics (e.g., anthracycline, gentamicin, cefalotin, vancomycin, telavancin, daptomycin, azithromycin, erythromycin, rocithromycin, furazolidone, amoxicillin, ampicillin, carbenicillin, flucloxacillin, methicillin, penicillin, ciprofloxacin, moxifloxacin, ofloxacin, doxycycline, minocycline, oxytetracycline, tetracycline, streptomycin, rifabutin, ethambutol, rifaximin, etc.), antiviral drugs (e.g., abacavir, acyclovir, ampligen, cidofovir, delavirdine, didanosine, efavirenz, entecavir, fosfonet, ganciclovir, ibacitabine, imunovir, idoxuridine, inosine, lopinavir, methisazone, nexavir, nevirapine, oseltamivir, penciclovir, stavudine, trifluridine, truvada, valaciclovir, zanamivir, etc.), daunorubicin hydrochloride, daunomycin, rubidomycin, cerubidine, idarubicin, doxorubicin, epirubicin and morpholino derivatives, phenoxizone biscyclopeptides (e.g., dactinomycin), basic glycopeptides (e.g., bleomycin), anthraquinone glycosides (e.g., plicamycin, mithramycin), anthracenediones (e.g., mitoxantrone), azirinopyrrolo indolediones (e.g., mitomycin), macrocyclic immunosuppressants (e.g., cyclosporine, FK-506, tacrolimus, prograf, rapamycin etc.), navelbene, CPT-11, anastrazole, letrazole, capecitabine, reloxafine, cyclophosphamide, ifosamide, droloxafine, allocolchicine, Halichondrin B, colchicine, colchicine derivatives, maytansine, rhizoxin, paclitaxel, paclitaxel derivatives, docetaxel, thiocolchicine, trityl cysterin, vinblastine sulfate, vincristine sulfate, cisplatin, carboplatin, hydroxyurea, N-methylhydrazine, epidophyllotoxin, procarbazine, mitoxantrone, leucovorin, and tegafur. “Taxanes” include paclitaxel, as well as any active taxane derivative or pro-drug. Chemotherapeutic agents such as erlotinib (TARCEVA®, Genentech/OSI Pharm.), bortezomib (VELCADE®, Millenium Pharm.), fulvestrant (FASLODEX®, AstraZeneca), sutent (SU11248, Pfizer), letrozole (FEMARA®, Novartis), imatinib mesylate (GLEEVEC®, Novartis), PTK787/ZK 222584 (Novartis), oxaliplatin (Eloxatin®, Sanofi), 5-FU (5-fluorouracil), leucovorin, Rapamycin (Sirolimus, RAPAMUNE®, Wyeth), lapatinib (TYKERB®, GSK572016, GlaxoSmithKline), lonafarnib (SCH 66336), sorafenib (BAY43-9006, Bayer Labs.), and gefitinib (TRESSA®, AstraZeneca), AG1478, AG1571 (SU 5271; Sugen), alkylating agents such as thiotepa and CYTOXAN® (cyclosphosphamide); alkyl sulfonates such as busulfan, improsulfan and piposulfan; antifolate antineoplastic such as pemetrexed (ALIMTA®, Eli Lilly), aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics, calicheamicin, calicheamicin gamma1I and calicheamicin omegaI1; dynemicin, including dynemicin A; bisphosphonates, such as clodronate; an esperamicin; as well as neocarzinostatin chromophore and related chromoprotein enediyne antibiotic chromophores, aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, adriamycin, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; kreskin (polysaccharide-K); razoxane; rhizoxin; sizofuran; spirogermanium; tenuazonic acid; triaziquone; 2,2′,2′″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.), ABRAXANE® Cremophor-free, albumin, nanoparticle formulation of paclitaxel (American Pharmaceutical Partners, Schaumberg, Ill.), and TAXOTERE® (doxetaxel; Rhone-Poulenc Rorer, Antony, France); chloranbucil; GEMZAR® (gemcitabine); 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

The terms “treat,” “treating” or “treatment” as used herein include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

Compounds, (including, but not limited to non-natural amino acids, non-natural amino acid polypeptides, modified non-natural amino acid polypeptides, and reagents for producing the aforementioned compounds) presented herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

Some of the compounds herein (including, but not limited to non-natural amino acids, non-natural amino acid polypeptides and modified non-natural amino acid polypeptides, and reagents for producing the aforementioned compounds) have asymmetric carbon atoms and can therefore exist as enantiomers or diastereomers. Diasteromeric mixtures can be separated into their individual diastereomers on the basis of their physical chemical differences by methods known, for example, by chromatography and/or fractional crystallization. Enantiomers can be separated by converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomers, enantiomers, and mixtures thereof are considered as part of the compositions described herein.

In additional or further embodiments, the compounds described herein (including, but not limited to non-natural amino acids, non-natural amino acid polypeptides and modified non-natural amino acid polypeptides, and reagents for producing the aforementioned compounds) are used in the form of pro-drugs. In additional or further embodiments, the compounds described herein ((including, but not limited to non-natural amino acids, non-natural amino acid polypeptides and modified non-natural amino acid polypeptides, and reagents for producing the aforementioned compounds) are metabolized upon administration to an organism in need to produce a metabolite that is then used to produce a desired effect, including a desired therapeutic effect. In further or additional embodiments are active metabolites of non-natural amino acids and “modified or unmodified” non-natural amino acid polypeptides.

The methods and formulations described herein include the use of N-oxides, crystalline forms (also known as polymorphs), or pharmaceutically acceptable salts of non-natural amino acids, non-natural amino acid polypeptides and modified non-natural amino acid polypeptides. In certain embodiments, non-natural amino acids, non-natural amino acid polypeptides and modified non-natural amino acid polypeptides may exist as tautomers. All tautomers are included within the scope of the non-natural amino acids, non-natural amino acid polypeptides and modified non-natural amino acid polypeptides presented herein. In addition, the non-natural amino acids, non-natural amino acid polypeptides and modified non-natural amino acid polypeptides described herein can exist in unsolvated as well as solvated forms with pharmaceutically acceptable solvents such as water, ethanol, and the like. The solvated forms of the non-natural amino acids, non-natural amino acid polypeptides and modified non-natural amino acid polypeptides presented herein are also considered to be disclosed herein.

Some of the compounds herein (including, but not limited to non-natural amino acids, non-natural amino acid polypeptides and modified non-natural amino acid polypeptides and reagents for producing the aforementioned compounds) may exist in several tautomeric forms. All such tautomeric forms are considered as part of the compositions described herein. Also, for example all enol-keto forms of any compounds (including, but not limited to non-natural amino acids, non-natural amino acid polypeptides and modified non-natural amino acid polypeptides and reagents for producing the aforementioned compounds) herein are considered as part of the compositions described herein.

Some of the compounds herein (including, but not limited to non-natural amino acids, non-natural amino acid polypeptides and modified non-natural amino acid polypeptides and reagents for producing either of the aforementioned compounds) are acidic and may form a salt with a pharmaceutically acceptable cation. Some of the compounds herein (including, but not limited to non-natural amino acids, non-natural amino acid polypeptides and modified non-natural amino acid polypeptides and reagents for producing the aforementioned compounds) can be basic and accordingly, may form a salt with a pharmaceutically acceptable anion. All such salts, including di-salts are within the scope of the compositions described herein and they can be prepared by conventional methods. For example, salts can be prepared by contacting the acidic and basic entities, in either an aqueous, non-aqueous or partially aqueous medium. The salts are recovered by using at least one of the following techniques: filtration, precipitation with a non-solvent followed by filtration, evaporation of the solvent, or, in the case of aqueous solutions, lyophilization.

Pharmaceutically acceptable salts of the non-natural amino acid polypeptides disclosed herein may be formed when an acidic proton present in the parent non-natural amino acid polypeptides either is replaced by a metal ion, by way of example an alkali metal ion, an alkaline earth ion, or an aluminum ion; or coordinates with an organic base. In addition, the salt forms of the disclosed non-natural amino acid polypeptides can be prepared using salts of the starting materials or intermediates. The non-natural amino acid polypeptides described herein may be prepared as a pharmaceutically acceptable acid addition salt (which is a type of a pharmaceutically acceptable salt) by reacting the free base form of non-natural amino acid polypeptides described herein with a pharmaceutically acceptable inorganic or organic acid. Alternatively, the non-natural amino acid polypeptides described herein may be prepared as pharmaceutically acceptable base addition salts (which are a type of a pharmaceutically acceptable salt) by reacting the free acid form of non-natural amino acid polypeptides described herein with a pharmaceutically acceptable inorganic or organic base.

The type of pharmaceutical acceptable salts, include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like.

The corresponding counterions of the non-natural amino acid polypeptide pharmaceutical acceptable salts may be analyzed and identified using various methods including, but not limited to, ion exchange chromatography, ion chromatography, capillary electrophoresis, inductively coupled plasma, atomic absorption spectroscopy, mass spectrometry, or any combination thereof. In addition, the therapeutic activity of such non-natural amino acid polypeptide pharmaceutical acceptable salts may be tested using the techniques and methods described in examples 87-91.

It should be understood that a reference to a salt includes the solvent addition forms or crystal forms thereof, particularly solvates or polymorphs. Solvates contain either stoichiometric or non-stoichiometric amounts of a solvent and are often formed during the process of crystallization with pharmaceutically acceptable solvents such as water, ethanol, and the like. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The screening and characterization of non-natural amino acid polypeptide pharmaceutical acceptable salts polymorphs and/or solvates may be accomplished using a variety of techniques including, but not limited to, thermal analysis, x-ray diffraction, spectroscopy, vapor sorption, and microscopy. Thermal analysis methods address thermochemical degradation or thermophysical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTTR, UVIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

### INTRODUCTION

Antibody-based therapeutics have emerged as important components of therapies for an increasing number of human malignancies in such fields as oncology, immunology, inflammatory and infectious diseases. In most cases, the basis of the therapeutic function is the high degree of specificity and affinity the antibody-based drug has for its target antigen. Arming monoclonal antibodies with drugs, toxins, or radionuclides is yet another strategy by which monoclonal antibodies may induce therapeutic effect. By combining the exquisite targeting specificity of antibody with the tumor killing power of toxic effector molecules, immunoconjugates permit sensitive discrimination between target and normal tissue thereby resulting in fewer side effects than most conventional chemotherapeutic drugs. The toxins utilized can specifically, stably and irreversibly conjugate to unique sites in the antibody. This unique process of conjugation allows for the precise control of the location of the toxin on the antibody, and also the number of toxins conjugated to each antibody. Both of these features are critical for controlling biophysical characteristics and toxicities associated with ADCs. (See for example Jackson et al., 2014, Tian et al., 2014).

Currently ADCs are advancing the field of cancer therapeutics and a number of ADCs targeting various agents have been approved or are in clinical trials. However, ADCs face challenges due to lack of therapeutic index and toxicity. The linker technology for attachment of the cytotoxic drug to an antibody impacts the stability of ADCs during the systemic circulation. The release of free drug in the circulation instead of the release inside the antigen expressing cancer cells can cause ADC potency loss, insufficient immunogenic cancer cell death, and increased toxicity. Therefore, there is a need to design a stable linker such for drug design and antibody conjugation.

Cluster of differentiation 70 (CD70) is frequently expressed in various malignant diseases, including clear cell renal carcinoma (RCC), with incidence ranges reported between 70% to 100%, while minimally expressed in normal tissues. The present disclosure provides a next-generation ADC using a technology platform whereby a CD70-specific monoclonal antibody is conjugated with AS269, a potent cytotoxic tubulin inhibitor. Site-specificity, high homogeneity, and stable covalent conjugation of the ADC leads to enhanced stability and pharmacokinetics, which may contribute to the lower systemic toxicity and increased targeted delivery of payload to tumor cells at a lower effective dose compared to other CD70 ADCs. The ADC of the present disclosure was designed to inhibit the growth of CD70 overexpressing cells through multiple sequential steps, including binding to CD70 on the surface of cancer cells, rapidly internalizing, trafficking to the lysosome, and metabolizing inside the lysosome to release pAF-AS269, which binds to microtubules and induces cancer cell cycle arrest and cell death.

Thus, in some aspects, the present disclosure provides next-generation, site-specific anti-CD70 ADC comprising a humanized CD70 targeting monoclonal antibody (mAb) conjugated to a cytotoxic tubulin inhibitor using a nonnatural amino acid incorporation technology platform. The ADC thus synthesized can be homogeneous and highly stable, thereby leading to a wider therapeutic window by delivering drug to target tumor cells with higher efficiency, maximizing on-target efficacy and minimizing off-target toxicity.

In some aspects, the ADC comprises a short, noncleavable hydroxylamine-PEG4 linker attached to the N-terminus of monomethyl auristatin F (MMAF) to produce a cytotoxic tublin inhibitor linker derivative AS269, which is a cytotoxic payload. MMAF is a highly potent synthetic auristatin derivative that inhibits cellular proliferation by disrupting tubulin polymerization. In some cases, the ADC contains two AS269 cytotoxic payloads site-specifically conjugated to anti-CD70 antibody comprising non-natural amino acids. In some cases, the ADC may display anti-tumor activity by optimizing the number and position of the payloads and the chemical bonds that conjugate the payloads to the antibody. In some cases, AS269 is a generally non-cell permeable tubulin inhibitor specifically designed to form a highly stable covalent bond with an antibody and kill tumor cells only upon entry into the cell when aided by the conjugated targeting antibody, thereby limiting off-target effects on healthy tissue. In some cases, AS269 displays limited to no permeability to cross the cell membrane without being conjugated with the targeting antibody, thereby reducing off-target toxicity. In some cases, AS269 is a poor substrate for multidrug resistance pumps (MDRs), thereby retaining and enriching the drug inside the cancer cell, which might lead to more potent killing of cancer cells. Combining AS269's unique characteristics with an optimized number and position of the payloads and the chemical bonds that conjugate the payloads to the antibody with a drug-to-antibody ratio (DAR) of 2 can result in enhanced in vivo stability, potency and low payload exposure in serum, which in turn may contribute to the ADC's observed anti-tumor activity and tolerability profile. In some cases, an ADC comprises a structure synthesized according to FIG. 1. In some cases, the ADC comprises AS269 as the payload, and an anti-CD70 antibody comprising one or more unnatural amino acids disclosed herein. In some embodiments, the AS269 payload is specifically and stably conjugated to the non-natural amino acid pAF on unique sites in the heavy chains of the mAb (one payload per heavy chain), the anti-CD70 antibody comprising the heavy chain with corresponding amino acid sequence SEQ ID NO: 3 with non-natural amino acid pAF at position A114 (Kabat numbering), and a light chain sequence with corresponding amino acid sequence SEQ ID NO: 2.

### Non-Natural Amino Acids

The present disclosure provides antibodies, antibody fragments or variants comprising at least one non-naturally encoded amino acid. Introduction of at least one non-naturally encoded amino acid into an antibody can allow for the application of conjugation chemistries that involve specific chemical reactions with one or more non-naturally encoded amino acids while not reacting with the commonly occurring 20 amino acids.

Non-naturally encoded amino acid site selection was based on surface exposure/site accessibility within the antibody and hydrophobic or neutral amino acid sites were selected to maintain the charge on the antibody. Methods for introducing non-natural amino acids inserted into sites in a protein are described for example in WO2018223108, WO2010/011735 and in WO2005/074650. The present disclosure employs such methodologies and techniques. The non-natural amino acids used in the methods and compositions described herein have at least one of the following four properties: (1) at least one functional group on the sidechain of the non-natural amino acid has at least one characteristics and/or activity and/or reactivity orthogonal to the chemical reactivity of the 20 common, genetically-encoded amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), or at least orthogonal to the chemical reactivity of the naturally occurring amino acids present in the polypeptide that includes the non-natural amino acid; (2) the introduced non-natural amino acids are substantially chemically inert toward the 20 common, genetically-encoded amino acids; (3) the non-natural amino acid can be stably incorporated into a polypeptide, preferably with the stability commensurate with the naturally-occurring amino acids or under typical physiological conditions, and further preferably such incorporation can occur via an in vivo system; and (4) the non-natural amino acid includes an oxime functional group or a functional group that can be transformed into an oxime group by reacting with a reagent, preferably under conditions that do not destroy the biological properties of the polypeptide that includes the non-natural amino acid (unless of course such a destruction of biological properties is the purpose of the modification/transformation), or where the transformation can occur under aqueous conditions at a pH between about 4 and about 8, or where the reactive site on the non-natural amino acid is an electrophilic site. Any number of non-natural amino acids can be introduced into the polypeptide. Non-natural amino acids may also include protected or masked oximes or protected or masked groups that can be transformed into an oxime group after deprotection of the protected group or unmasking of the masked group. Non-natural amino acids may also include protected or masked carbonyl groups, which can be transformed into a carbonyl group after deprotection of the protected group or unmasking of the masked group and thereby are available to react with hydroxylamines or aminooxy groups to form oxime groups. Oxime-based non-natural amino acids may be synthesized by methods well known in the art, (see for example WO2013/185117 and WO2005/074650), including reaction of a carbonyl-containing non-natural amino acid with a hydroxylamine- or aminooxy-containing reagent.

In some embodiments, non-naturally encoded amino acid site selection is based on surface exposure. Example, one possible site is an amino acid having a solvent accessible surface area ratio of 30% or more, 40% or more, 50% or more, 60% or more, 70% or more, 80% or more, 90% or more, or 95% or more. In some embodiments, one possible site is an amino acid having a solvent accessible surface area ratio of about 30%, about 40%, about 50%, about 60%, about 70%, about 80%, about 90%, or about 95%, or more. The solvent accessible surface area can be calculated based on the DSSP program [Biopolymers, 22, 2577-2637 (1983)], using a crystalline structure analyzing data file of antibodies or antibody fragments registered in Protein data bank (PDB).

The ratio of the solvent accessible surface area of the amino acid residues of interest can be calculated by dividing the antibody structural solvent accessible surface area calculated in the above by the solvent accessible surface area of alanine-X-alanine (X represents the amino acid residues of interest). In this connection, there is a case in which two or more PDB files are present on one species of protein, and any one of them can be used in the present invention.

Alternatively, the solvent accessibility of an amino acid can be determined by a solvent accessibility test in which a functional group on the amino acid (a thiol, amino, or carbonyl group) is functionalized when treated with an electrophilic reagent or a nucleophilic reagent, or the like. Based on the test results, the functional group (i.e., the thiol, amino, or carbonyl group) can be called, for example, at least 50% solvent accessible when at least 50% of the functional group is functionalized in the test. In some embodiments, the non-naturally encoded amino acid site is at least 30%, at least 40%, at least 50%, at least 60%, at least 70%, at least 75%, at least 80%, at least 85%, at least 90%, or at least 95% solvent accessible. Examples of solvent accessibility test include, but are not limited to, propargylation of a surface thiol group, or α-bromopyruvate reacting with a surface thiol group, etc.

In some embodiments disclosed herein are antibodies comprising one or more non-naturally encoded amino acids. The one or more non-natural amino acids may be encoded by a codon that does not code for one of the twenty natural amino acids. The one or more non-natural amino acids may be encoded by a nonsense codon (stop codon). The stop codon may be an amber codon. The amber codon may comprise a UAG sequence. The stop codon may be an ochre codon. The ochre codon may comprise a UAA sequence. The stop codon may be an opal or umber codon. The opal or umber codon may comprise a UGA sequence. The one or more non-natural amino acids may be encoded by a four-base codon.

Non-natural amino acids of the present disclosure include, but are not limited to, 1) substituted phenylalanine and tyrosine analogues, such as 4-amino-L-phenylalanine, 4-acetyl-L-phenylalanine (pAF), 4-azido-L-phenylalanine, 4-nitro-L-phenylalanine, 3-methoxy-L-phenylalanine, 4-isopropyl-L-phenylalanine, 3-nitro-L-tyrosine, O-methyl-L-tyrosine and 0-phosphotyrosine; 2) amino acids that can be photo-cross-linked, e.g., amino acids with aryl azide or benzophenone groups, such as 4-azidophenylalanine or 4-benzoylphenylalanine; 3) amino acids that have unique chemical reactivity, such as 4-acetyl-L-phenylalanine, 3-acetyl-L-phenylalanine, O-allyl-L-tyrosine, O-2-propyn-1-yl-L-tyrosine, N-(ethylthio)thiocarbonyl-L-phenylalanine and p-(3-oxobutanoyl)-L-phenylalanine; 4) heavy-atom-containing amino acids, e.g., for phasing in X-ray crystallography, such as 4-iodo-L-phenylalanine or 4-bromo-L-phenylalanine; 5) a redox-active amino acid, such as 3,4-dihydroxy-L-phenylalanine; 6) a fluorinated amino acid, such as a 2-fluorophenylalanine (e.g., 2-fluoro-L-phenylalanine), a 3-fluorophenylalanine (e.g., 3-fluoro-L-phenylalanine) or a 4-fluorophenylalanine (e.g., 4-fluoro-L-phenylalanine; 7) a fluorescent amino acid, such as an amino acid containing a naphthyl, dansyl or 7-aminocoumarin side chain; 8) a photocleavable or photoisomerizable amino acid, such as an amino acid comprising an azobenzyl or nitrobenzyl, e.g., cysteine, serine or tyrosine comprising azobenzyl or nitrobenzyl; 9) a β-amino acid (e.g., a β² or β³ amino acid); 10) a homo-amino acid, such as homoglutamine (e.g., beta-homoglutamine) or homophenylalanine (e.g., beta-homophenylalanine); 11) a proline or pyruvic acid derivative; 12) a 3-substituted alanine derivative; 14) a glycine derivative; 15) a linear core amino acid; 16) a diamino acid; 17) a D-amino acid; 18) an N-methyl amino acid; 19) a phosphotyrosine mimetic, such as a carboxymethylphenylalanine (pCmF) (e.g., 4-carboxymethyl-L-phenylalanine); 20) 2-aminooctanoic acid; and 21) an amino acid comprising a saccharide moiety, such as N-acetyl-L-glucosaminyl-L-serine, beta-N-acetylglucosamine-O-serine, N-acetyl-L-galactosaminyl-L-serine, alpha-N-acetylgalactosamine-O-serine, O-(3-O-D-galactosyl-N-acetyl-beta-D-galactosaminyl)-L-serine, N-acetyl-L-glucosaminyl-L-threonine, alpha-N-acetylgalactosamine-O-threonine, 3-O—(N-acetyl-beta-D-glucosaminyl)-L-threonine, N-acetyl-L-glucosaminyl-L-asparagine, N4-(β-N-Acetyl-D-glucosaminyl)-L-asparagine and O-(mannosyl)-L-serine; an amino acid wherein the naturally-occurring N- or O-linkage between the amino acid and the saccharide is replaced by a covalent linkage not commonly found in nature, including but not limited to, an alkene, an oxime, a thioether, an amide and the like; or an amino acid containing saccharides that are not commonly found in naturally-occurring polypeptides, such as 2-deoxy-glucose, 2-deoxy-galactose and the like. Specific examples of non-natural amino acids include, but are not limited to, a p-acetylphenylalanine (4-acetyl phenylalanine), 4-acetyl-L-phenylalanine, also referred to herein as p-acetyl-L-phenylalanine (pAF), a 4-boronophenylalanine (pBoF) (e.g., 4-borono-L-phenylalanine, a 4-propargyloxyphenylalanine (pPrF) (e.g., 4-propargyloxy-L-phenylalanine), an O-methyltyrosine (e.g., O-methyl-L-tyrosine), a 3-(2-naphthyl)alanine (NapA) (e.g., 3-(2-naphthyl)-L-alanine), a 3-methylphenylalanine (e.g., 3-methyl-L-phenylalanine), an O-allyltyrosine (e.g., O-allyl-L-tyrosine), an O-isopropyltyrosine (e.g., O-isopropyl-L-tyrosine), a dopamine (e.g., L-Dopa), a 4-isopropylphenylalanine (e.g., 4-isopropyl-L-phenylalanine), a 4-azidophenylalanine (pAz) (e.g., 4-azido-L-phenylalanine), a 4-benzoylphenylalanine (pBpF) (e.g., 4-benzoyl-L-phenylalanine), an O-phosphoserine (e.g., O-phospho-L-serine), an O-phosphotyrosine (e.g., O-phospho-L-tyrosine), a 4-iodophenylalanine (pIF) (e.g., 4-iodo-L-phenylalanine, a 4-bromophenylalanine (e.g., 4-bromo-L-phenylalanine), a 4-aminophenylalanine (e.g., 4-amino-L-phenylalanine), a 4-cyanophenylalanine (pCNF) (e.g., 4-cyano-L-phenylalanine, a (8-hydroxyquinolin-3-yl)alanine (HQA) (e.g., (8-hydroxyquinolin-3-yl)-L-alanine), a (2,2-bipyridin-5-yl)alanine (BipyA) (e.g., (2,2-bipyridin-5-yl)-L-alanine), and the like. Additional non-natural amino acids are disclosed in Liu et al. (2010) Annu Rev Biochem, 79:413-44; Wang et al. (2005) Angew Chem Int Ed, 44:34-66; and Published International Application Nos.: WO 2012/166560, WO 2012/166559, WO 2011/028195, WO 2010/037062, WO 2008/083346, WO 2008/077079, WO 2007/094916, WO 2007/079130, WO 2007/070659 and WO 2007/059312, the entire contents of each of which are hereby incorporated by reference herein in their entirety. In some embodiments, the one or more non-natural amino acids can be p-acetylphenylalanine. In some more particular embodiments, the one or more non-natural amino acids can be p-acetyl-L-phenylalanine (pAF).

In some embodiments, one or more non-natural amino acids is selected from the group consisting of 4-acetyl phenylalanine, 3-O—(N-acetyl-beta-D-glucosaminyl)threonine, N4-(β-N-Acetyl-D-glucosaminyl)asparagine, O-allyltyrosine, alpha-N-acetylgalactosamine-O-serine, alpha-N-acetylgalactosamine-O-threonine, 2-aminooctanoic acid, 2-aminophenylalanine, 3-aminophenylalanine, 4-aminophenylalanine, 2-aminotyrosine, 3-aminotyrosine, 4-azidophenylalanine, 4-benzoylphenylalanine, (2,2-bipyridin-5yl)alanine, 3-boronophenylalanine, 4-boronophenylalanine, 4-bromophenylalanine, p-carboxymethylphenylalanine, 4-carboxyphenylalanine, p-cyanophenylalanine, 3,4-dihydroxyphenylalanine, 4-ethynylphenylalanine, 2-fluorophenylalanine, 3-fluorophenylalanine, 4-fluorophenylalanine, O-(3-O-D-galactosyl-N-acetyl-beta-D-galactosaminyl)serine, homoglutamine, (8-hydroxyquinolin-3-yl)alanine, 4-iodophenylalanine, 4-isopropylphenylalanine, O-i-propyltyrosine, 3-isopropyltyrosine, O-mannopyranosylserine, 2-methoxyphenylalanine, 3-methoxyphenylalanine, 4-methoxyphenylalanine, 3-methylphenylalanine, O-methyltyrosine, 3-(2-naphthyl)alanine, 5-nitrohistidine, 4-nitrohistidine, 4-nitroleucine, 2-nitrophenylalanine, 3-nitrophenylalanine, 4-nitrophenylalanine, 4-nitrotryptophan, 5-nitrotryptophan, 6-nitrotryptophan, 7-nitrotryptophan, 2-nitrotyrosine, 3-nitrotyrosine, O-phosphoserine, O-phosphotyrosine, 4-propargyloxyphenylalanine, O-2-propyn-1-yltyrosine, 4-sulfophenylalanine and O-sulfotyrosine.

In some further embodiments, one or more non-natural amino acids is selected from the group consisting of 4-acetyl-L-phenylalanine (para-acetyl-L-phenylalanine (pAF)), 3-O—(N-acetyl-beta-D-glucosaminyl)-L-threonine, N4-(β-N-Acetyl-D-glucosaminyl)-L-asparagine, O-allyl-L-tyrosine, alpha-N-acetylgalactosamine-O-L-serine, alpha-N-acetylgalactosamine-O-L-threonine, 2-aminooctanoic acid, 2-amino-L-phenylalanine, 3-amino-L-phenylalanine, 4-amino-L-phenylalanine, 2-amino-L-tyrosine, 3-amino-L-tyrosine, 4-azido-L-phenylalanine, 4-benzoyl-L-phenylalanine, (2,2-bipyridin-5yl)-L-alanine, 3-borono-L-phenylalanine, 4-borono-L-phenylalanine, 4-bromo-L-phenylalanine, p-carboxymethyl-L-phenylalanine, 4-carboxy-L-phenylalanine, p-cyano-L-phenylalanine, 3,4-dihydroxy-L-phenylalanine (L-DOPA), 4-ethynyl-L-phenylalanine, 2-fluoro-L-phenylalanine, 3-fluoro-L-phenylalanine, 4-fluoro-L-phenylalanine, O-(3-O-D-galactosyl-N-acetyl-beta-D-galactosaminyl)-L-serine, L-homoglutamine, (8-hydroxyquinolin-3-yl)-L-alanine, 4-iodo-L-phenylalanine, 4-isopropyl-L-phenylalanine, O-i-propyl-L-tyrosine, 3-isopropyl-L-tyrosine, 0-mannopyranosyl-L-serine, 2-methoxy-L-phenylalanine, 3-methoxy-L-phenylalanine, 4-methoxy-L-phenylalanine, 3-methyl-L-phenylalanine, O-methyl-L-tyrosine, 3-(2-naphthyl)-L-alanine, 5-nitro-L-histidine, 4-nitro-L-histidine, 4-nitro-L-leucine, 2-nitro-L-phenylalanine, 3-nitro-L-phenylalanine, 4-nitro-L-phenylalanine, 4-nitro-L-tryptophan, 5-nitro-L-tryptophan, 6-nitro-L-tryptophan, 7-nitro-L-tryptophan, 2-nitro-L-tyrosine, 3-nitro-L-tyrosine, O-phospho-L-serine, O-phospho-L-tyrosine, 4-propargyloxy-L-phenylalanine, O-2-propyn-1-yl-L-tyrosine, 4-sulfo-L-phenylalanine and O-sulfo-L-tyrosine. In some embodiments, the one or more non-natural amino acids can be be p-acetyl-L-phenylalanine (pAF). Thus, in some embodiments, each and every one of the one or more non-natural amino acids is pAF.

In certain embodiments of the disclosure, an antibody with at least one non-natural amino acid includes at least one post-translational modification. In one embodiment, the at least one post-translational modification comprises attachment of a molecule including but not limited to a biologically active agent such as a drug, including a small molecule drug, or any other desirable compound or substance, comprising a second reactive group to at least one non-natural amino acid comprising a first reactive group utilizing chemistry methodology that is known to one of ordinary skill in the art to be suitable for the particular reactive groups. For example, the first reactive group is a keto moiety (including but not limited to, the non-natural amino acid p-acetyl-phenylalanine, or more particularly, p-acetyl-L-phenylalanine (pAF) and the second reactive group is an aminooxy moiety. In another example, the first reactive group is the azido moiety (including but not limited to, the non-natural amino acid p-azido-L-phenylalanine) and the second reactive group is the alkynyl moiety. In certain embodiments of the modified antibody polypeptide of the present disclosure at least one non-natural amino acid, (including but not limited to, non-natural amino acid containing a keto functional group), comprising at least one post-translational modification is used where the at least one post-translational modification comprises a saccharide moiety. In certain embodiments, the post-translational modification is made in vivo in a eukaryotic cell or in a non-eukaryotic cell. In other embodiments the post-translational modification is made in vitro. In another embodiment, the post-translational modification is made in vitro and in vivo.

In some embodiments, the non-natural amino acid may be modified to incorporate a chemical group. In some embodiments the non-natural amino acid may be modified to incorporate a ketone group. The one or more non-natural amino acids may comprise at least one carbonyl.

In some embodiments disclosed herein the non-natural amino acid is site-specifically incorporated into the antibody, antibody fragment or variant. In some embodiments the non-natural amino acid is site-specifically incorporated into an antibody, antibody fragment or variant. Methods for incorporating a non-natural amino acid into a molecule, for example, proteins, polypeptides or peptides, are disclosed in U.S. Pat. Nos. 7,332,571; 7,928,163; 7,696,312; 8,008,456; 8,048,988; 8,809,511; 8,859,802; 8,791,231; 8,476,411; or 9,637,411, (each of which is incorporated herein by reference in its entirety), and in the Examples herein. The one or more non-natural amino acids may be incorporated by methods known in the art. For example, cell-based or cell-free systems may be used, and auxotrophic strains may also be used in place of engineered tRNA and synthetase. In certain embodiments, orthogonal tRNA synthetase are used as disclosed in, for example, WO2002085923A2; WO2002086075A2; WO2004035743A2; WO2007021297A1; WO2006068802A2; and WO2006069246A2; the contents of each of which are incorporated herein by reference in their entirety. Incorporating one or more non-natural amino acids into the antibody or antibody fragment or variant may comprise modifying one or more amino acid residues in the antibody or antibody fragment or variant. Modifying the one or more amino acid residues in the antibody or antibody fragment or variant may comprise mutating one or more nucleotides in the nucleotide sequence encoding the antibody or antibody fragment or variant. Mutating the one or more nucleotides in the nucleotide sequence encoding the antibody or antibody fragment or variant may comprise altering a codon encoding an amino acid to a nonsense codon. Incorporating one or more non-natural amino acids into the antibody or antibody fragment or variant may comprise modifying one or more amino acid residues in the antibody or antibody fragment or variant to produce one or more amber codons in the antibody or antibody fragment or variant. The one or more non-natural amino acids may be incorporated into the antibody or antibody fragment or variant in response to an amber codon. The one or more non-natural amino acids may be site-specifically incorporated into the antibody or antibody fragment or variant. Incorporating one or more non-natural amino acids into the antibody or antibody fragment or variant may comprise one or more genetically encoded non-natural amino acids with orthogonal chemical reactivity relative to the canonical twenty amino acids to site-specifically modify the biologically active molecule or targeting agent. Incorporating the one or more non-natural amino acids may comprise use of a tRNA/aminoacyl-tRNA synthetase pair to site-specifically incorporate one or more non-natural amino acids at defined sites in the biologically active molecule or targeting agent in response to one or more amber nonsense codon. Additional methods for incorporating non-natural amino acids include, but are not limited to, methods disclosed in Chatterjee et al., A Versatile Platform for Single- and Multiple-Unnatural Amino Acid Mutagenesis in Escherichia coli, Biochemistry, 2013; Kazane et al., J Am Chem Soc, 135(1):340-6, 2013; Kim et al., J Am Chem Soc, 134(24):9918-21, 2012; Johnson et al., Nat Chem Biol, 7(11):779-86, 2011; and Hutchins et al., J Mol Biol, 406(4):595-603, 2011. The one or more non-natural amino acids may be produced through selective reaction of one or more natural amino acids. The selective reaction may be mediated by one or more enzymes. In non-limiting examples, the selective reaction of one or more cysteines with formylglycine generating enzyme (FGE) may produce one or more formylglycines as described in Rabuka et al., Nature Protocols 7: 1052-1067, 2012. The one or more non-natural amino acids may involve a chemical reaction to form a linker. The chemical reaction to form the linker may include a bioorthogonal reaction. The chemical reaction to form the linker may include click chemistry. See for example WO2006/050262 incorporated herein by reference in its entirety.

Any position of the antibody or antibody fragment is suitable for selection to incorporate a non-natural amino acid, and selection may be based on rational design or by random selection for any or no particular desired purpose. Selection of desired sites may be based on producing a non-natural amino acid polypeptide (which may be further modified or remain unmodified) having any desired property or activity, including but not limited to a receptor binding modulators, receptor activity modulators, modulators of binding to binder partners, binding partner activity modulators, binding partner conformation modulators, dimer or multimer formation, no change to activity or property compared to the native molecule, or manipulating any physical or chemical property of the polypeptide such as solubility, aggregation, or stability. Alternatively, the sites identified as critical to biological activity may also be good candidates for substitution with a non-natural amino acid, again depending on the desired activity sought for the polypeptide. Another alternative would be to simply make serial substitutions in each position on the polypeptide chain with a non-natural amino acid and observe the effect on the activities of the polypeptide. Any means, technique, or method for selecting a position for substitution with a non-natural amino acid into any polypeptide is suitable for use in the methods, techniques and compositions described herein.

The structure and activity of naturally-occurring mutants of a polypeptide that contain deletions can also be examined to determine regions of the protein that are likely to be tolerant of substitution with a non-natural amino acid. Once residues that are likely to be intolerant to substitution with non-natural amino acids have been eliminated, the impact of proposed substitutions at each of the remaining positions can be examined using methods including, but not limited to, the three-dimensional structure of the relevant polypeptide, and any associated ligands or binding proteins. X-ray crystallographic and NMR structures of many polypeptides are available in the Protein Data Bank (PDB, see world wide web for resb.org), a centralized database containing three-dimensional structural data of large molecules of proteins and nucleic acids, one can be used to identify amino acid positions that can be substituted with non-natural amino acids. In addition, models may be made investigating the secondary and tertiary structure of polypeptides, if three-dimensional structural data is not available. Thus, the identity of amino acid positions that can be substituted with non-natural amino acids can be readily obtained.

Exemplary sites of incorporation of a non-natural amino acid include, but are not limited to, those that are excluded from potential receptor binding regions, or regions for binding to binding proteins or ligands may be fully or partially solvent exposed, have minimal or no hydrogen-bonding interactions with nearby residues, may be minimally exposed to nearby reactive residues, and/or may be in regions that are highly flexible as predicted by the three-dimensional crystal structure of a particular polypeptide with its associated receptor, ligand or binding proteins.

A wide variety of non-natural amino acids can be substituted for, or incorporated into, a given position in a polypeptide. By way of example, a particular non-natural amino acid may be selected for incorporation based on an examination of the three-dimensional crystal structure of a polypeptide with its associated ligand, receptor and/or binding proteins, a preference for conservative substitutions.

The synthesis of p-acetyl-(+/−)-phenylalanine and m-acetyl-(+/−)-phenylalanine is described in Zhang, Z., et al., Biochemistry 42: 6735-6746 (2003), and the synthesis of p-acetyl-L-phenylalainine is described in WO2015/153761A2; the entire contents of each of which are hereby incorporated by reference in their entirety. Other carbonyl-containing amino acids can be similarly prepared.

### Anti-CD70 Antibodies

The present invention provides novel ADCs comprising antibodies, antibody fragments or variants thereof engineered to have one, or more non-naturally encoded amino acids incorporated at any desired position in the heavy and/or light chain amino acid sequence. Further, the present invention provides ADCs comprising one or more antibodies, antibody fragments or variants thereof engineered to have one, or more non-naturally encoded amino acids site specifically incorporated in the heavy and/or light chain amino acid sequence conjugated to drug or payload via a phosphate-based linker. In some embodiments, the antibody, antibody fragment or variant thereof binds to a tumor-associated CD70 antigen. In some embodiments the invention provides anti-CD70 ADCs comprising antibodies, antibody fragments or variants thereof engineered to have one, or more non-naturally encoded amino acids incorporated at any desired position in the heavy and/or light chain amino acid sequence. In some embodiments, the present invention provides anti-CD70 ADCs comprising one or more antibodies, antibody fragments or variants thereof engineered to have one, or more non-naturally encoded amino acids site specifically incorporated in the heavy and/or light chain amino acid sequence conjugated to drug or payload via a linker.

Antibody or antibody fragments or variants of the disclosure may be human, humanized, engineered, non-human, and/or chimeric antibody or antibody fragments. An antibody or antibody fragment or variant provided herein may comprise two or more amino acid sequences. A first amino acid sequence may comprise a first antibody chain and a second amino acid sequence may comprise a second antibody chain. A first antibody chain may comprise a first amino acid sequence, and a second antibody chain may comprise a second amino acid sequence. A chain of an antibody may refer to an antibody heavy chain, an antibody light chain, or a combination of a region or all of an antibody heavy chain and a region or all of an antibody light chain. As a non-limiting example, an antibody provided herein comprises a heavy chain or fragment or variant thereof, and a light chain or fragment or variant thereof. Two amino acid sequences of an antibody, including two antibody chains, may be connected, attached, or linked by one or more disulfide bonds, a chemical linker, a peptide linker, or a combination thereof. A chemical linker includes a linker via a non-natural amino acid. A chemical linker includes a linker via one or more non-natural amino acids. A chemical linker can include a chemical conjugate. A peptide linker includes any amino acid sequence joining the two amino acid sequences. The peptide linker may comprise 1 or more, 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, 100 or more amino acids. The peptide linker may be a portion of any antibody, including a domain of an antibody, such as a variable domain, CH1, CH2, CH3, and/or CL domain. In some embodiments a heavy and a light chain are connected, attached, or linked, for example, via a peptide linker. In some cases, a heavy chain and a light chain are connected, for example, by one or more disulfide bonds.

Antibodies, antibody fragments and antibody variants of the disclosure may interact or engage with an antigen on an effector cell. The effector cell can include, but is not limited to, an immune cell, a genetically modified cell having increase or decrease cytotoxic activity, a cell involved in the host defense mechanism, an anti-inflammatory cell, a leukocyte, a lymphocyte, a macrophage, an erythrocyte, a thrombocyte, a neutrophil, a monocyte, an eosinophil, a basophil, a mast cell, a NK cell, a B-cell, or a T-cell. In some embodiments the immune cell may be a T cell such as a cytotoxic T cell or natural killer T cell. The antibody or antibody fragment may interact with a receptor on a T-cell such as, but not limited to a T-cell receptor (TCR). The TCR may comprise TCR alpha, TCR beta, TCR gamma, and/or TCR delta or TCR zeta. Antibody or antibody fragments of the disclosure may bind to a receptor on a lymphocyte, dendritic cell, B-cell, macrophage, monocytes, neutrophils and/or NK cells. Antibody or antibody fragments of the disclosure may bind to a cell surface receptor. Antibody or antibody fragments of the disclosure may bind to an antigen receptor, such as for example, a CD70 antigen receptor. Antibody or antibody fragments of the disclosure can be conjugated to a T-cell surface antigen.

Some cell surface antigens have a high overexpression pattern in a large number of tumors, making them excellent targets in the development of ADCs. Thus, the present disclosure provides novel anti-CD70 antibodies, or the corresponding antibody fragments, and antibody-drug conjugates thereof for use as therapeutic agents. Disclosed herein are novel anti-CD70 antibodies, antibody fragments or variants thereof, each having a non-naturally encoded amino acid that facilitate antibody conjugation to a drug (e.g., a drug, payload, toxin molecule) or drug-linker compound.

Antibodies, antibody fragments or variants provided in the present disclosure may be human, humanized, engineered, non-human, and/or chimeric antibody or antibody fragments that bind to the extracellular domain of the target antigen, which can be overexpressed in a number of cancers. Thus, novel antibodies, compositions and antibody drug conjugates for the treatment and/or diagnosis of antigen-expressing cancers are beneficial, including but not limited to CD70-expressing cancers.

Antibodies or antibody fragments or variants disclosed herein include, but are not limited to, analogs, isoforms, mimetics, fragments, or hybrids of CD70. Antibodies or antibody fragments or variants of CD70 of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like.

Antibodies comprising non-natural amino acids are also disclosed herein. In certain embodiments, the antibody or antibody fragments or variants include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like. In some embodiments, the anti-CD70, antibody or antibody fragments or variants comprises one or more non-naturally encoded amino acids.

Non-limiting examples of antibodies or antibody fragments or variants of the present disclosure comprise the sequences listed in Table 1.

In certain embodiments antibody or antibody fragments disclosed herein are anti-CD70 antibodies or antibody fragments or variants thereof. In certain embodiments, the anti-CD70 antibodies or antibody fragments or variants disclosed herein can be humanized. Anti-CD70 antibodies or antibody fragments or variants disclosed herein include, but are not limited to, CD70 analogs, isoforms, mimetics, fragments, or hybrids. Anti-CD70 antibodies or antibody fragments or variants of the present disclosure include but are not limited to Fv, Fc, Fab, and (Fab′)₂, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, bispecific antibodies and the like. The anti-CD70 antibodies or antibody fragments or variants of the present disclosure can contain one or more polypeptide chain (e.g., one or more heavy chain and/or light chain), and can be characterized by the amino acid sequence(s) of the one or more polypeptide chain. The anti-CD70 antibodies or antibody fragments or variants of the present disclosure comprise an amino acid sequence of SEQ ID NOs: 1 to 9 (Table 1). The antibodies, fragments or variants of the present disclosure can be an anti-CD70 antibody, fragment or variant. In certain embodiments, the anti-CD70 antibody comprises a heavy chain and light chain amino acid sequence selected from the group consisting of SEQ ID NOs: 1 to 9. In certain embodiments, the anti-CD70 antibody consists of a heavy chain and light chain amino acid sequence selected from the group consisting of SEQ ID NOs: 1 to 9. In certain embodiments, the anti-CD70 antibody comprises a heavy chain amino acid sequence of any one of SEQ ID NOs: 1, 3, 4 and 5; and a light chain amino acid sequence of any one of SEQ ID NOs: 2, 6, 7, 8 and 9. In certain embodiments, the anti-CD70 antibody comprises a heavy chain, wherein the heavy chain amino acid sequence is SEQ ID NO: 3; and a light chain, wherein the light chain amino acid sequence is SEQ ID NO: 2.

The present disclosure provides an anti-CD70 ADC that is a humanized monoclonal antibody drug conjugate which functions, by non-limiting example, by promoting cell survival and expansion of antigen primed CD8 T cells, formation of memory T cells and proliferation of B cells. CD70 receptors have been found in high densities in cancer tissue, for example 34000-189000 copies per cell (Caki-1, 786-0, L-428, UMRC3, LP-1, DBTRG-05 MG) (McDonagh, C. F., Engineered anti-CD70 antibody-drug conjugate with increased therapeutic index, Molecular Cancer Therapeutics, 7(9):2913-2923 (2008)) and in non-cancerous tissue and/or normal tissue the receptors are on 5%-15% of activated T cells and 10%-25% of activated B cells. CD70 expression has been found on ~40% of multiple myeloma isolates (Preclinical Characterization of SGN-70, a Humanized Antibody Directed against CD70, Cancer Therapy: Preclinical, 2008) and confirmed CD70 expression on a high percentage of, for example, Hodgkin lymphoma Reed-Sternberg cells, non-Hodgkin lymphoma, and renal cell carcinoma tumors. CD70 is a type II integral membrane protein of the TNF family. For purposes of this invention, it is possible for the anti-CD70 antibody to be any known CD70 antibody with one non-naturally encoded amino acid. For purposes of illustration, anti-CD70 antibodies are described in Table 1.

In one embodiment of the present invention, the ADC comprises a heavy chain, wherein the heavy chain amino acid sequence is SEQ ID NO: 3 with one non-naturally encoded amino acid at position A114 (Kabat numbering). In some embodiments, the non-naturally encoded amino acid is para-acetyl-L-phenylalanine (pAF). In another embodiment of the present invention, the antibody comprises a light chain, wherein the light chain amino acid sequence is SEQ ID NO: 2.

In some other embodiments of the present invention, the anti-CD70 antibody comprises a heavy chain having an amino acid sequence with one or more non-naturally encoded amino acids, wherein the heavy chain amino acid sequence shares at least 90% identity with SEQ ID NO: 1. In other embodiments, the anti-CD70 antibody comprises a light chain having an amino acid sequence optionally containing one or more non-naturally encoded amino acids, wherein the light chain amino acid sequence shares at least 90% identity with SEQ ID NO: 2. In other embodiments, the anti-CD70 antibody comprises a heavy chain having an amino acid sequence with one or more non-naturally encoded amino acids, wherein the heavy chain amino acid sequence shares at least 90% identity with SEQ ID NO: 3. In other embodiments, the anti-CD70 antibody comprises a heavy chain having an amino acid sequence with one or more non-naturally encoded amino acids, wherein the heavy chain amino acid sequence shares at least 90% identity with SEQ ID NO: 4. In other embodiments, the anti-CD70 antibody comprises a light chain having an amino acid sequence with one or more non-naturally encoded amino acids, wherein the light chain amino acid sequence shares at least 90% identity with SEQ ID NO: 5. In other embodiments, the anti-CD70 antibody comprises a light chain having an amino acid sequence with one or more non-naturally encoded amino acids, wherein the light chain amino acid sequence shares at least 90% identity with SEQ ID NO: 6. In other embodiments, the anti-CD70 antibody comprises a light chain having an amino acid sequence with one or more non-naturally encoded amino acids, wherein the light chain amino acid sequence shares at least 90% identity with SEQ ID NO: 7. In other embodiments, the anti-CD70 antibody comprises a light chain having an amino acid sequence with one or more non-naturally encoded amino acids, wherein the light chain amino acid sequence shares at least 90% identity with SEQ ID NO: 8. In other embodiments, the anti-CD70 antibody comprises a light chain having an amino acid sequence with one or more non-naturally encoded amino acids, wherein the light chain amino acid sequence shares at least 90% identity with SEQ ID NO: 9.

In some other embodiments of the present invention, the anti-CD70 antibody comprises a heavy chain having an amino acid sequence with one or more non-naturally encoded amino acids, wherein the heavy chain amino acid sequence shares at least 95% identity with SEQ ID NO: 1. In other embodiments, the anti-CD70 antibody comprises a light chain having an amino acid sequence optionally containing one or more non-naturally encoded amino acids, wherein the light chain amino acid sequence shares at least 95% identity with SEQ ID NO: 2. In other embodiments, the anti-CD70 antibody comprises a heavy chain having an amino acid sequence with one or more non-naturally encoded amino acids, wherein the heavy chain amino acid sequence shares at least 95% identity with SEQ ID NO: 3. In other embodiments, the anti-CD70 antibody comprises a heavy chain having an amino acid sequence with one or more non-naturally encoded amino acids, wherein the heavy chain amino acid sequence shares at least 95% identity with SEQ ID NO: 4. In other embodiments, the anti-CD70 antibody comprises a light chain having an amino acid sequence with one or more non-naturally encoded amino acids, wherein the light chain amino acid sequence shares at least 95% identity with SEQ ID NO: 5. In other embodiments, the anti-CD70 antibody comprises a light chain having an amino acid sequence with one or more non-naturally encoded amino acids, wherein the light chain amino acid sequence shares at least 95% identity with SEQ ID NO: 6. In other embodiments, the anti-CD70 antibody comprises a light chain having an amino acid sequence with one or more non-naturally encoded amino acids, wherein the light chain amino acid sequence shares at least 95% identity with SEQ ID NO: 7. In other embodiments, the anti-CD70 antibody comprises a light chain having an amino acid sequence with one or more non-naturally encoded amino acids, wherein the light chain amino acid sequence shares at least 95% identity with SEQ ID NO: 8. In other embodiments, the anti-CD70 antibody comprises a light chain having an amino acid sequence with one or more non-naturally encoded amino acids, wherein the light chain amino acid sequence shares at least 95% identity with SEQ ID NO: 9.

In some other embodiments of the present invention, the anti-CD70 antibody comprises a heavy chain having an amino acid sequence with one or more non-naturally encoded amino acids, wherein the heavy chain amino acid sequence shares at least 96% identity with SEQ ID NO: 1. In other embodiments, the anti-CD70 antibody comprises a light chain having an amino acid sequence optionally containing one or more non-naturally encoded amino acids, wherein the light chain amino acid sequence shares at least 96% identity with SEQ ID NO: 2. In other embodiments, the anti-CD70 antibody comprises a heavy chain having an amino acid sequence with one or more non-naturally encoded amino acids, wherein the heavy chain amino acid sequence shares at least 96% identity with SEQ ID NO: 3. In other embodiments, the anti-CD70 antibody comprises a heavy chain having an amino acid sequence with one or more non-naturally encoded amino acids, wherein the heavy chain amino acid sequence shares at least 96% identity with SEQ ID NO: 4. In other embodiments, the anti-CD70 antibody comprises a light chain having an amino acid sequence with one or more non-naturally encoded amino acids, wherein the light chain amino acid sequence shares at least 96% identity with SEQ ID NO: 5. In other embodiments, the anti-CD70 antibody comprises a light chain having an amino acid sequence with one or more non-naturally encoded amino acids, wherein the light chain amino acid sequence shares at least 96% identity with SEQ ID NO: 6. In other embodiments, the anti-CD70 antibody comprises a light chain having an amino acid sequence with one or more non-naturally encoded amino acids, wherein the light chain amino acid sequence shares at least 96% identity with SEQ ID NO: 7. In other embodiments, the anti-CD70 antibody comprises a light chain having an amino acid sequence with one or more non-naturally encoded amino acids, wherein the light chain amino acid sequence shares at least 96% identity with SEQ ID NO: 8. In other embodiments, the anti-CD70 antibody comprises a light chain having an amino acid sequence with one or more non-naturally encoded amino acids, wherein the light chain amino acid sequence shares at least 96% identity with SEQ ID NO: 9.

In some other embodiments of the present invention, the anti-CD70 antibody comprises a heavy chain having an amino acid sequence with one or more non-naturally encoded amino acids, wherein the heavy chain amino acid sequence shares at least 97% identity with SEQ ID NO: 1. In other embodiments, the anti-CD70 antibody comprises a light chain having an amino acid sequence optionally containing one or more non-naturally encoded amino acids, wherein the light chain amino acid sequence shares at least 97% identity with SEQ ID NO: 2. In other embodiments, the anti-CD70 antibody comprises a heavy chain having an amino acid sequence with one or more non-naturally encoded amino acids, wherein the heavy chain amino acid sequence shares at least 97% identity with SEQ ID NO: 3. In other embodiments, the anti-CD70 antibody comprises a heavy chain having an amino acid sequence with one or more non-naturally encoded amino acids, wherein the heavy chain amino acid sequence shares at least 97% identity with SEQ ID NO: 4. In other embodiments, the anti-CD70 antibody comprises a light chain having an amino acid sequence with one or more non-naturally encoded amino acids, wherein the light chain amino acid sequence shares at least 97% identity with SEQ ID NO: 5. In other embodiments, the anti-CD70 antibody comprises a light chain having an amino acid sequence with one or more non-naturally encoded amino acids, wherein the light chain amino acid sequence shares at least 97% identity with SEQ ID NO: 6. In other embodiments, the anti-CD70 antibody comprises a light chain having an amino acid sequence with one or more non-naturally encoded amino acids, wherein the light chain amino acid sequence shares at least 97% identity with SEQ ID NO: 7. In other embodiments, the anti-CD70 antibody comprises a light chain having an amino acid sequence with one or more non-naturally encoded amino acids, wherein the light chain amino acid sequence shares at least 97% identity with SEQ ID NO: 8. In other embodiments, the anti-CD70 antibody comprises a light chain having an amino acid sequence with one or more non-naturally encoded amino acids, wherein the light chain amino acid sequence shares at least 97% identity with SEQ ID NO: 9.

In some other embodiments of the present invention, the anti-CD70 antibody comprises a heavy chain having an amino acid sequence with one or more non-naturally encoded amino acids, wherein the heavy chain amino acid sequence shares at least 98% identity with SEQ ID NO: 1. In other embodiments, the anti-CD70 antibody comprises a light chain having an amino acid sequence optionally containing one or more non-naturally encoded amino acids, wherein the light chain amino acid sequence shares at least 98% identity with SEQ ID NO: 2. In other embodiments, the anti-CD70 antibody comprises a heavy chain having an amino acid sequence with one or more non-naturally encoded amino acids, wherein the heavy chain amino acid sequence shares at least 98% identity with SEQ ID NO: 3. In other embodiments, the anti-CD70 antibody comprises a heavy chain having an amino acid sequence with one or more non-naturally encoded amino acids, wherein the heavy chain amino acid sequence shares at least 98% identity with SEQ ID NO: 4. In other embodiments, the anti-CD70 antibody comprises a light chain having an amino acid sequence with one or more non-naturally encoded amino acids, wherein the light chain amino acid sequence shares at least 98% identity with SEQ ID NO: 5. In other embodiments, the anti-CD70 antibody comprises a light chain having an amino acid sequence with one or more non-naturally encoded amino acids, wherein the light chain amino acid sequence shares at least 98% identity with SEQ ID NO: 6. In other embodiments, the anti-CD70 antibody comprises a light chain having an amino acid sequence with one or more non-naturally encoded amino acids, wherein the light chain amino acid sequence shares at least 98% identity with SEQ ID NO: 7. In other embodiments, the anti-CD70 antibody comprises a light chain having an amino acid sequence with one or more non-naturally encoded amino acids, wherein the light chain amino acid sequence shares at least 98% identity with SEQ ID NO: 8. In other embodiments, the anti-CD70 antibody comprises a light chain having an amino acid sequence with one or more non-naturally encoded amino acids, wherein the light chain amino acid sequence shares at least 98% identity with SEQ ID NO: 9.

In some other embodiments of the present invention, the anti-CD70 antibody comprises a heavy chain having an amino acid sequence with one or more non-naturally encoded amino acids, wherein the heavy chain amino acid sequence shares at least 99% identity with SEQ ID NO: 1. In other embodiments, the anti-CD70 antibody comprises a light chain having an amino acid sequence optionally containing one or more non-naturally encoded amino acids, wherein the light chain amino acid sequence shares at least 99% identity with SEQ ID NO: 2. In other embodiments, the anti-CD70 antibody comprises a heavy chain having an amino acid sequence with one or more non-naturally encoded amino acids, wherein the heavy chain amino acid sequence shares at least 99% identity with SEQ ID NO: 3. In other embodiments, the anti-CD70 antibody comprises a heavy chain having an amino acid sequence with one or more non-naturally encoded amino acids, wherein the heavy chain amino acid sequence shares at least 99% identity with SEQ ID NO: 4. In other embodiments, the anti-CD70 antibody comprises a light chain having an amino acid sequence with one or more non-naturally encoded amino acids, wherein the light chain amino acid sequence shares at least 99% identity with SEQ ID NO: 5. In other embodiments, the anti-CD70 antibody comprises a light chain having an amino acid sequence with one or more non-naturally encoded amino acids, wherein the light chain amino acid sequence shares at least 99% identity with SEQ ID NO: 6. In other embodiments, the anti-CD70 antibody comprises a light chain having an amino acid sequence with one or more non-naturally encoded amino acids, wherein the light chain amino acid sequence shares at least 99% identity with SEQ ID NO: 7. In other embodiments, the anti-CD70 antibody comprises a light chain having an amino acid sequence with one or more non-naturally encoded amino acids, wherein the light chain amino acid sequence shares at least 99% identity with SEQ ID NO: 8. In other embodiments, the anti-CD70 antibody comprises a light chain having an amino acid sequence with one or more non-naturally encoded amino acids, wherein the light chain amino acid sequence shares at least 99% identity with SEQ ID NO: 9.

In some other embodiments of the present invention, the anti-CD70 antibody comprises a heavy chain having an amino acid sequence, wherein the heavy chain amino acid sequence shares 100% identity with SEQ ID NO: 1. In other embodiments, the anti-CD70 antibody comprises a light chain having an amino acid sequence optionally containing one or more non-naturally encoded amino acids, wherein the one or more non-naturally encoded amino acids replaces one or more amino acids of a light chain amino acid sequence shares 100% identity with SEQ ID NO: 2. In other embodiments, the anti-CD70 antibody comprises a heavy chain having an amino acid sequence with one or more non-naturally encoded amino acids, wherein the heavy chain amino acid sequence shares 100% identity with SEQ ID NO: 3. In other embodiments, the anti-CD70 antibody comprises a heavy chain having an amino acid sequence with one or more non-naturally encoded amino acids, wherein the heavy chain amino acid sequence shares 100% identity with SEQ ID NO: 4. In other embodiments, the anti-CD70 antibody comprises a light chain having an amino acid sequence with one or more non-naturally encoded amino acids, wherein the light chain amino acid sequence shares 100% identity with SEQ ID NO: 5. In other embodiments, the anti-CD70 antibody comprises a light chain having an amino acid sequence with one or more non-naturally encoded amino acids, wherein the light chain amino acid sequence shares 100% identity with SEQ ID NO: 6. In other embodiments, the anti-CD70 antibody comprises a light chain having an amino acid sequence with one or more non-naturally encoded amino acids, wherein the light chain amino acid sequence shares 100% identity with SEQ ID NO: 7. In other embodiments, the anti-CD70 antibody comprises a light chain having an amino acid sequence with one or more non-naturally encoded amino acids, wherein the light chain amino acid sequence shares 100% identity with SEQ ID NO: 8. In other embodiments, the anti-CD70 antibody comprises a light chain having an amino acid sequence with one or more non-naturally encoded amino acids, wherein the light chain amino acid sequence shares 100% identity with SEQ ID NO: 9.

In some preferred embodiments of the present invention, the antibody is an anti-CD70 antibody comprising (i) a light chain having an amino acid sequence, wherein the light chain amino acid sequence shares 100% identity with SEQ ID NO: 2, and (ii) a heavy chain having an amino acid sequence with one non-naturally encoded amino acid, wherein the heavy chain amino acid sequence shares 100% identity with SEQ ID NO: 3, wherein the non-naturally encoded amino acid is para-acetyl-L-phenylalanine (pAF). In some other preferred embodiments of the present invention, the anti-CD70 antibody comprises (i) two light chains, each having amino acid sequence, wherein each light chain amino acid sequence shares 100% identity with SEQ ID NO: 2, and (ii) two heavy chains, each having an amino acid sequence, wherein each heavy chain amino acid sequence has one non-naturally encoded amino acid and shares 100% identity with SEQ ID NO: 3, wherein each non-naturally encoded amino acid is para-acetyl-L-phenylalanine (pAF). Non-naturally encoded amino acid site selection is described within this specification, and as it specifically pertains to anti-CD70 antibodies, sites were selected based on surface exposure/site accessibility within the antibody and hydrophobic/neutral amino acid sites were selected to maintain the charge on the antibody.

Table 1. Anti-CD70 heavy chain (HC) and light chain (LC) amino acid sequences with Amber sites for non-natural amino acid (nnAA) incorporation. Also disclosed are: all of the sequences in Table 1, wherein X is replaced by any nnAA; all of the sequences in Table 1, wherein any amino acid is replaced by any nnAA; all of the sequences in Table 1, wherein X is pAF; all of the heavy chain sequences in Table 1, wherein a non-naturally encoded amino acid is site specifically incorporated at position 114, according to Kabat numbering, as well known to the skilled artisan; and and all of the heavy chain sequences in Table 1, wherein EEM is replaced with DEL. WT: Wild Type; HC: Heavy Chain; LC: Light Chain; X denotes nnAA.

| SEQ ID NO | Description | Amino Acid Sequence |
|---|---|---|
| 1 | Anti-CD70 | EVQLVQSGAEVKKPGATVKISCKVSGYTFTDYY |
| | Heavy chain 1 | MNWVQQAPGKGLEWMGIINPYNGGTHYNQKFK |
| | | GRVTITADTSTDTAYMELSSLRSEDTAVYYCATS |
| | | GYDLYFDYWGQGTLVTVSSASTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSREEMTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPG |
| 2 | Anti-CD70 | EIVMTQSPATLSVSPGERATLSCKASQNVGTAVA |
| | Light chain | WYQQKPGQAPRLLIYSAFNRYNGIPARFSGSGPG |
| | WT | TDFTLTISSLQSEDFAVYYCQQYSTYPLTFGGGTK |
| | | VEIKRTVAAPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| 3 | Anti-CD70 | EVQLVQSGAEVKKPGATVKISCKVSGYTFTDYY |
| | Heavy Chain | MNWVQQAPGKGLEWMGIINPYNGGTHYNQKFK |
| | nnAA @ A114 | GRVTITADTSTDTAYMELSSLRSEDTAVYYCATS |
| | | GYDLYFDYWGQGTLVTVSSXSTKGPSVFPLAPSS |
| | | KSTSGGTAALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSREEMTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPG |
| 4 | Anti-CD70 | EVQLVQSGAEVKKPGATVKISCKVSGYTFTDYY |
| | Heavy Chain | MNWVQQAPGKGLEWMGIINPYNGGTHYNQKFK |
| | nnAA @ A136 | GRVTITADTSTDTAYMELSSLRSEDTAVYYCATS |
| | | GYDLYFDYWGQGTLVTVSSASTKGPSVFPLAPSS |
| | | KSTSGGTXALGCLVKDYFPEPVTVSWNSGALTSG |
| | | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSREEMTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPG |
| 5 | Anti-CD70 | EVQLVQSGAEVKKPGATVKISCKVSGYTFTDYY |
| | Heavy Chain | MNWVQQAPGKGLEWMGIINPYNGGTHYNQKFK |
| | nnAA @ L159 | GRVTITADTSTDTAYMELSSLRSEDTAVYYCATS |
| | | GYDLYFDYWGQGTLVTVSSASTKGPSVFPLAPSS |
| | | KSTSGGTAALGCLVKDYFPEPVTVSWNSGAXTSG |
| | | VHTFPAVLQSSGLYSLSSVVTVPSSSLGTQTYICN |
| | | VNHKPSNTKVDKKVEPKSCDKTHTCPPCPAPELL |
| | | GGPSVFLFPPKPKDTLMISRTPEVTCVVVDVSHED |
| | | PEVKFNWYVDGVEVHNAKTKPREEQYNSTYRVV |
| | | SVLTVLHQDWLNGKEYKCKVSNKALPAPIEKTIS |
| | | KAKGQPREPQVYTLPPSREEMTKNQVSLTCLVKG |
| | | FYPSDIAVEWESNGQPENNYKTTPPVLDSDGSFFL |
| | | YSKLTVDKSRWQQGNVFSCSVMHEALHNHYTQK |
| | | SLSLSPG |
| 6 | Anti-CD70 | EIVMTQSPATLSVSPGERATLSCKASQNVGTAVA |
| | Light Chain | WYQQKPGQAPRLLIYSAFNRYNGIPARFSGSGPG |
| | nnAA @ V110 | TDFTLTISSLQSEDFAVYYCQQYSTYPLTFGGGTK |
| | | VEIKRTXAAPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| 7 | Anti-CD70 | EIVMTQSPATLSVSPGERATLSCKASQNVGTAVA |
| | Light Chain | WYQQKPGQAPRLLIYSAFNRYNGIPARFSGSGPG |
| | nnAA @A112 | TDFTLTISSLQSEDFAVYYCQQYSTYPLTFGGGTK |
| | | VEIKRTVAXPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| 8 | Anti-CD70 | EIVMTQSPATLSVSPGERATLSCKASQNVGTAVA |
| | Light Chain | WYQQKPGQAPRLLIYSAFNRYNGIPARFSGSGPG |
| | nnAA @ S114 | TDFTLTISSLQSEDFAVYYCQQYSTYPLTFGGGTK |
| | | VEIKRTVAAPXVFIFPPSDEQLKSGTASVVCLLNN |
| | | FYPREAKVQWKVDNALQSGNSQESVTEQDSKDS |
| | | TYSLSSTLTLSKADYEKHKVYACEVTHQGLSSPV |
| | | TKSFNRGEC |
| 9 | Anti-CD70 | EIVMTQSPATLSVSPGERATLSCKASQNVGTAVA |
| | Light Chain | WYQQKPGQAPRLLIYSAFNRYNGIPARFSGSGPG |
| | nnAA @ S121 | TDFTLTISSLQSEDFAVYYCQQYSTYPLTFGGGTK |
| | | VEIKRTVAAPSVFIFPPXDEQLKSGTASVVCLLNN |
| | | FYPREAKVQWKVDNALQSGNSQESVTEQDSKDS |
| | | TYSLSSTLTLSKADYEKHKVYACEVTHQGLSSPV |
| | | TKSFNRGEC |

In some aspects, the present disclosure provides an isolated anti-CD70 antibody or fragment thereof comprising at least one amino acid sequence selected from the group consisting of the sequences listed in Table 1. In some embodiments, there is provided an isolated anti-CD70 antibody or fragment thereof consisting of at least one amino acid sequence selected from the group consisting of the sequences listed in Table 1.

In some embodiments, there is provided an isolated anti-CD70 antibody or fragment thereof that shares 100% sequence identity with the amino acid sequence of SEQ ID NO: 2.

In some embodiments, there is provided an isolated anti-CD70 antibody or fragment thereof that shares 100% sequence identity with the amino acid sequence of SEQ ID NO: 3.

In some embodiments, there is provided an isolated anti-CD70 antibody or fragment thereof having a light chain sharing 100% sequence identity with amino acid sequence of SEQ ID NO: 2 and a heavy chain that shares 100% sequence identity with the amino acid sequence of SEQ ID NO: 3.

In some embodiments, there is provided an isolated anti-CD70 antibody or fragment thereof that shares 100% sequence identity with the amino acid sequence of SEQ ID NO: 4.

In some embodiments, there is provided an isolated anti-CD70 antibody or fragment thereof that shares 100% identity with the amino acid sequence of SEQ ID NO: 5.

In some embodiments, there is provided an isolated anti-CD70 antibody or fragment thereof that shares 100% identity with the amino acid sequence of SEQ ID NO: 6.

In some embodiments, there is provided an isolated anti-CD70 antibody or fragment thereof that shares 100% sequence identity with the amino acid sequence of SEQ ID NO: 7.

In some embodiments, there is provided an isolated anti-CD70 antibody or fragment thereof that shares 100% sequence identity with the amino acid sequence of SEQ ID NO: 8.

In some embodiments, there is provided an isolated anti-CD70 antibody or fragment thereof that shares 100% sequence identity with the amino acid sequence of SEQ ID NO: 9.

In some embodiments, there is provided a nucleic acid encoding any one of SEQ ID NOs: 1 to 9.

In some embodiments, there is provided a nucleic acid encoding any one of SEQ ID NOs: 3 to 9. In some embodiments, there is provided a nucleic acid encoding SEQ ID NO: 3.

In some general aspects, the present disclosure provides a vector comprising a nucleic acid encoding any one of SEQ ID NOs: 1 to 9. In some embodiments, the present disclosure provides a nucleic acid encoding any one of SEQ ID NOs: 3 to 9. In some embodiments, the present disclosure provides a nucleic acid encoding SEQ ID NO: 3.

### Drug-Linkers

In some aspects, the present disclosure relates to linkers for intracellular delivery of drug conjugates. Many procedures and linker molecules for attachment of various compounds to peptides are known. See, for example, European Patent Application No. 0188256; U.S. Pat. Nos. 4,671,958, 4,659,839, 4,414,148, 4,699,784, 4,680,338, 4,569,789 and 10,550,190; PCT Application Publication Nos. WO 2012/166559 A1, WO 2012/166560 A1, WO 2013/185117 A1, WO 2013/192360 A1 and WO 2022/040596 A1; and US Patent Application Publication No. US 2017/0182181 A1; the contents of each of which are hereby incorporated by reference in their entirety.

Methods for selecting and designing linkers are well known in the art. Linkers may be designed de novo, including by way of example only, as part of high-throughput screening process (in which case numerous polypeptides may be designed, synthesized, characterized and/or tested) or based on the interests of the researcher. The linker may also be designed based on the structure of a known or partially characterized polypeptide. The principles for selecting which amino acid(s) to substitute and/or modify and the choice of which modification to employ are described in WO2013/185117, for example. Linkers may be designed to meet the needs of the experimenter or end user. Such needs may include, but are not limited to, manipulating the therapeutic effectiveness of the polypeptide, improving the safety profile of the polypeptide, adjusting the pharmacokinetics, pharmacologics and/or pharmacodynamics of the polypeptide, such as, by way of example only, increasing water solubility, bioavailability, increasing serum half-life, increasing therapeutic half-life, modulating immunogenicity, modulating biological activity, or extending the circulation time. In addition, such modifications include, by way of example only, providing additional functionality to the polypeptide, incorporating an antibody, and any combination of the aforementioned modifications.

Generally, a linker of the present disclosure can be a unit that is combinable with one or more additional units, such that the combined linker units can bond to one or more drugs or payloads. Each linker unit can be comprised of one or more moieties, each of which may occur one or more times. Non-limiting examples of linker units can include bivalent —(CH₂CH₂—O)— moieties. A linker can further contain a reactive moiety, such as an aminooxy group. The reactive moiety can be joined, for example, at a far terminal end of a linker, and the drug can be joined, for example, at a near terminal end of the linker. Thus, the linker can act as a spacer or bridge between the drug and the reactive moiety.

Drugs with linkers containing an aminooxy group allow for reaction with a variety of electrophilic groups to form conjugates. Like hydrazines, hydrazides and semicarbazides, the enhanced nucleophilicity of the aminooxy group permits it to react efficiently and selectively with a variety of molecules that contain carbonyl groups, including but not limited to ketones. See, e.g., Shao, J. and Tam, J., J. Am. Chem. Soc. 117:3893-3899 (1995); H. Hang and C. Bertozzi, Acc. Chem. Res. 34(9): 727-736 (2001). An oxime results generally from the reaction of an aminooxy group with a carbonyl-containing group such as, by way of example, a ketone, such as an acyl group.

Thus, in certain embodiments described herein are drug-linkers comprising an aminooxy group. Such drug-linkers may be in the form of a salt or may be incorporated into a non-natural amino acid polypeptide, polymer, polysaccharide, or a polynucleotide and optionally post translationally modified.

In some embodiments, a linker as disclosed herein is connected to a drug, and is also connected to an antibody, antibody fragment or variant thereof, via a linkage or adduct moiety. Thus, the linker bridges the drug/payload and the antibody, antibody fragment or variant thereof.

In some aspects, the present disclosure provides a drug-linker/payload, wherein the drug or payload is a cytotoxic drug or agent. In some aspects of the disclosure, the cytotoxic drug is compound 6 having the following structure:

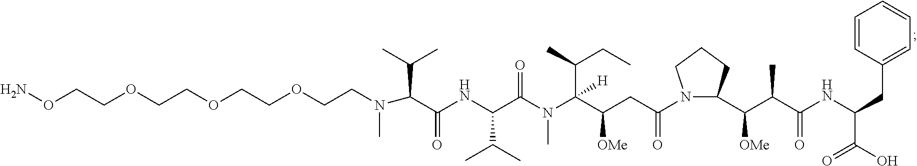 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)CCOCCOCCOCCON)C(C)C]

or a salt thereof.

Drug-linkers compounds such as compound 6 can be employed or conjugated with any targeting ligand such as an antibody or antibody fragment, that is selected based in its specificity for an antigen expressed on a target cell or at a target site of interest. The drug or payload linkers of the invention can be employed with antibody or antibody fragments to a variety of antigens including but not limited to tumor associated antigens, tumor specific antigens, cancer antigens or diseases specific antigens. In some embodiments, drug-linker compounds such as compound 6 can be employed with anti-CD70 antibody, antibody fragments or antibody drug conjugates of the invention. Synthesis of such drug-linkers are well known to the skilled artisan. See for example EP14874745, Dubowchik et al., Bioconjugate Chem. 13: 855-869, (2002); Doronina et al., Nature Biotechnology 21(7): 778-784, (2003); WO2012/166560; WO2013/185117 each incorporated herein by reference.

The present disclosure provides drug moieties with linkers that reduce the toxicity of the moiety in vivo while retaining pharmacological activity. In some embodiments, the toxicity of the linked drug, when administered to an animal or human, is reduced or eliminated compared to the free toxic group or toxic group derivatives comprising labile linkages, while retaining pharmacological activity. In some embodiments, increased doses of the linked drug group may be administered to animals or humans with greater safety. In certain embodiments, the non-natural amino acid polypeptides linked to a drug moiety provides in vitro and in vivo stability. In some embodiments, the non-natural amino acid polypeptides linked to a drug moiety are efficacious and less toxic compared to the free drug moiety.

### Antibody Drug Conjugates

Antibody drug conjugates (ADCs) of the present disclosure provide novel therapeutics by combining the selectivity of an antibody comprising one or more non-natural amino acids and a cytotoxic agent conjugated to the antibody. Targeted cytotoxic drug delivery into tumor tissue increases the therapeutic window of these agents considerably. ADCs of the present disclosure comprise an antibody bound to a cytotoxic drug via a linker. The stability of the linker between the antibody and the cytotoxic drug is essential for the ADC integrity in circulation. Successful ADC development for a given target antigen depends on optimization of antibody selection, linker design and stability, drug potency and mode of drug and linker conjugation to the antibody. Linker properties of pH and redox sensitivities and protease susceptibility influence circulatory stability and release of the drug moiety.

In some embodiments of the disclosure, the antibody of the ADC comprises a full length antibody or fragment thereof that binds to an antigen, and is conjugated to a cytotoxic agent or an immunosuppressive agent, wherein the antibody-drug conjugate exerts: (a) a cytotoxic or cytostatic effect on the antigen-expressing or antigen targeting cell, or (b) a cytotoxic, cytostatic, or immunosuppressive/immune activating effect on an antigen-expressing immune cell, wherein the conjugation occurs at a non-naturally encoded amino acid in the antibody. In some embodiments, the antigen of the antigen-expressing cell, antigen-targeting cell, or antigen-expressing immune cell, is CD70, but is not limited to such.

In some embodiments, the antibody, variant, or composition of the present disclosure may be an antibody, variant, or composition that binds to an antigen receptor. In other embodiments the antibody, variant, or composition may be an antibody, variant, or composition that binds to extracellular surface of an antigen receptor. In some embodiments the antibody, variant, or composition of the present disclosure may be an antibody, variant, or composition that has CDRs grafted onto the framework region of the variable region. In other embodiments the antibody, variant, or composition of the present disclosure may be an antibody, variant, or composition that has a non-naturally encoded amino acid. In some embodiments the antibody, variant, or composition may be an antibody, variant, or composition that is described by more than one of the embodiments elsewhere in the present disclosure. In some embodiments the antibody, antibody variant or antibody composition(s) disclosed herein may be fully humanized. In other embodiments the antibody, antibody variant or antibody composition(s) disclosed herein may be chimeric. In some embodiments the antibody may be an antibody that is a full length antibody (Variable+Fc regions), Fab, bispecific, Fab-dimers, Fab-bispecific, Fab-trispecific, bispecific T-cell engagers, dual-affinity re-targeting antibody, IgG1/IgG3 bispecific antibody, diabody, bispecific diabody, scFv-Fc, minibody.

In one embodiment, the ADC comprises an antibody conjugated to a drug wherein the conjugation occurs via a non-naturally encoded amino acid in the antibody. In one embodiment, the ADC comprises an antibody conjugated to a drug wherein the conjugation occurs via a non-naturally encoded amino acid in the heavy chain of the antibody. In one embodiment, the ADC comprises an antibody conjugated to a drug wherein the conjugation occurs via a non-naturally encoded amino acid in the light chain of the antibody. In one embodiment, the ADC comprises a full-length antibody conjugated to a drug wherein the conjugation occurs via a non-naturally encoded amino acid in the antibody. In one embodiment, the ADC comprises a full-length antibody conjugated to a drug wherein the conjugation occurs via a non-naturally encoded amino acid in the heavy chain of the antibody. In one embodiment, the ADC comprises a full-length antibody conjugated to a drug wherein the conjugation occurs via a non-naturally encoded amino acid in the light chain of the antibody.

In some embodiments, the drug of the ADC is a cytotoxic drug or agent. In some aspects of the invention, the cytotoxic drug is compound 6. In some embodiments the drug is generated as described in the Examples herein. In some embodiments, the ADC comprises an antibody, antibody fragment or variant thereof engineered to have one or more non-naturally encoded amino acids site specifically incorporated in the heavy and/or light chain amino acid sequence conjugated to drug via a linker.

In some embodiments, the present invention provides an anti-CD70 ADC, wherein the antibody is an anti-CD70 antibody comprising a light chain and a heavy chain, and wherein the antibody is conjugated to a drug via a non-naturally encoded amino acid. In some more particular embodiments, the anti-CD70 ADC comprises (a) an anti-CD70 antibody comprising (i) two light chain amino acid sequences, each amino acid sequence sharing 100% identity with SEQ ID NO: 2, and (ii) two heavy chain amino acid sequences, each amino acid sequence sharing 100% identity with SEQ ID NO: 3 with non-naturally encoded amino acid para-acetyl-L-phenylalanine (pAF) at position 114 (Kabat numbering); and (b) a drug, wherein the drug is conjugated to the anti-CD70 antibody via the pAF. In some even more particular embodiments, drug is conjugated to each pAF, such that the ADC comprises two drug payloads. Accordingly, the drug to antibody ratio (DAR) is about 2. In some embodiments, the drug is compound 6. In some embodiments, drug is conjugated to each pAF via an oxime bond. It is understood that the stoichiometry of the drug to antibody during a conjugation reaction may be less than 2, or a conjugation reaction between drug and antibody may be incomplete, resulting in a DAR of less than 2. Thus, the DAR can be a non-integer value, such as, for example, 1.1, 1.2, 1.3, 1.4, 1.5, 1.6, 1.7, 1.8 or 1.9.

In some aspects, the present disclosure provides an antibody-drug conjugate (ADC) comprising:
a drug-linker group having the structure:
   and

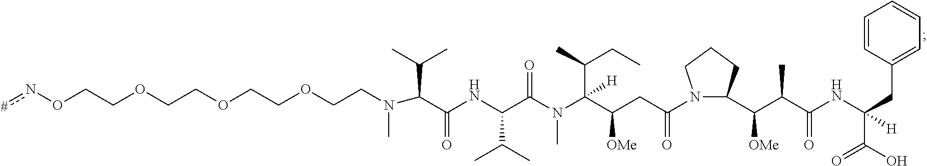

an anti-CD70 antibody or fragment thereof comprising one or more heavy chains; wherein at least one member of the one or more heavy chains has an amino acid sequence that (a) comprises SEQ ID NO: 3; or (b) shares at least 90% identity with SEQ ID NO: 3; wherein:
[symbol] represents a single bond or a double bond, and
#represents a connection to the anti-CD70 antibody or fragment thereof.

In some embodiments, at least one member of the one or more heavy chains has an amino acid sequence that shares at least 95%, at least 96%, at least 97%, at least 98% or at least 99% identity with SEQ ID NO: 3. In some embodiments, at least one member of the one or more heavy chains has the amino acid sequence of SEQ ID NO: 3. In some embodiments, at least one member of the one or more heavy chains is the amino acid sequence of SEQ ID NO: 3. In some embodiments, each of the one or more heavy chains is the amino acid sequence of SEQ ID NO: 3.

In some embodiments, the anti-CD70 antibody or fragment thereof comprises two heavy chains, wherein each heavy chain has an amino acid sequence that shares at least 90%, at least 95%, at least 96%, at least 97%, at least 98% or at least 99% identity with SEQ ID NO: 3. In some embodiments, each heavy chain has the amino acid sequence of SEQ ID NO: 3. In some embodiments, each heavy chain is the amino acid sequence of SEQ ID NO: 3.

In some embodiments, the anti-CD70 antibody or fragment thereof further comprises one or more light chains, wherein at least one of the one or more light chains has an amino acid sequence that shares at least 90% identity with SEQ ID NO: 2, 6, 7, 8 or 9. In some embodiments, at least one of the one or more light chains has an amino acid sequence that shares at least 90% identity with the amino acid sequence of SEQ ID NO: 2. In some embodiments, at least one of the one or more light chains has the amino acid sequence of SEQ ID NO: 2.

In some embodiments, the anti-CD70 antibody or fragment thereof comprises two light chains. In some embodiments, each light chain has an amino acid sequence that shares at least 90%, at least 95%, at least 96%, at least 97%, at least 98% or at least 99% identity with SEQ ID NO: 2. In some embodiments, the anti-CD70 antibody or fragment thereof comprises two light chains, wherein each light chain has the amino acid sequence of SEQ ID NO: 2.

In some other embodiments, at least one of the one or more light chains has an amino acid sequence containing a non-naturally encoded amino acid, wherein the amino acid sequence is selected from the group consisting of SEQ ID NO: 6, 7, 8 and 9.

In some embodiments, each [symbol] represents a double bond.

In some embodiments, a drug-linker group is one or more drug-linker groups.

In some aspects, the present disclosure provides an antibody-drug conjugate (ADC) comprising:
one or more drug-linker groups having the structure:

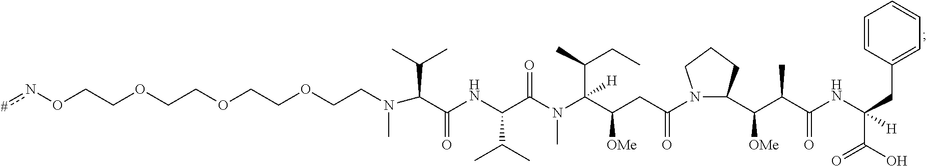

an anti-CD70 antibody or fragment thereof comprising one or more heavy chains, wherein at least one of the one or more heavy chains has an amino acid sequence containing a non-naturally encoded amino acid, wherein the amino acid sequence is selected from the group consisting of SEQ ID NO: 3, 4 and 5; wherein:
each [symbol] represents a single bond or a double bond that covalently joins one of the
one or more drug-linker groups to the anti-CD70 antibody or fragment thereof, and
each #represents a site of connection to the anti-CD70 antibody or fragment thereof.

In some embodiments, at least one of the one or more heavy chains has the amino acid sequence of SEQ ID NO: 3. In some embodiments, at least one of the one or more heavy chains is the amino acid sequence of SEQ ID NO: 3. In some embodiments, each of the one or more heavy chains is the amino acid sequence of SEQ ID NO: 3.

In some other embodiments, at least one of the one or more heavy chains has the amino acid sequence of SEQ ID NO: 4. In some other embodiments, at least one of the one or more heavy chains is the amino acid sequence of SEQ ID NO: 4. In some other embodiments, each of the one or more heavy chains is the amino acid sequence of SEQ ID NO: 4.

In some other embodiments, at least one of the one or more heavy chains has the amino acid sequence of SEQ ID NO: 5. In some other embodiments, at least one of the one or more heavy chains is the amino acid sequence of SEQ ID NO: 5. In some other embodiments, each of the one or more heavy chains is the amino acid sequence of SEQ ID NO: 5.

In some embodiments, the anti-CD70 antibody or fragment thereof comprises two heavy chains, wherein each heavy chain comprises one non-naturally encoded amino acid. In some embodiments, each heavy chain has the amino acid sequence of SEQ ID NO: 3.

In some other embodiments, the anti-CD70 antibody or fragment thereof comprises two heavy chains, wherein each heavy chain comprises one non-naturally encoded amino acid. In some embodiments, each heavy chain has the amino acid sequence of SEQ ID NO: 4.

In some other embodiments, the anti-CD70 antibody or fragment thereof comprises two heavy chains, wherein each heavy chain comprises one non-naturally encoded amino acid. In some embodiments, each heavy chain has the amino acid sequence of SEQ ID NO: 5.

In some embodiments, the anti-CD70 antibody or fragment thereof further comprises one or more light chains, wherein at least one of the one or more light chains has an amino acid sequence that shares at least 90% identity with SEQ ID NO: 2, 6, 7, 8 or 9. In some embodiments, at least one of the one or more light chains has an amino acid sequence that shares at least 90% identity with the amino acid sequence of SEQ ID NO: 2. In some embodiments, at least one of the one or more light chains has the amino acid sequence of SEQ ID NO: 2. In some embodiments, at least one of the one or more light chains is the amino acid sequence of SEQ ID NO: 2. In some embodiments, each of the one or more light chains is the amino acid sequence of SEQ ID NO: 2.

In some embodiments, the anti-CD70 antibody or fragment thereof comprises two light chains. In some embodiments, each light chain has an amino acid sequence, wherein each amino acid sequence shares at least 90%, at least 95%, at least 96%, at least 97%, at least 98% or at least 99% identity with SEQ ID NO: 2. In some embodiments, the anti-CD70 antibody or fragment thereof comprises two light chains, wherein each light chain has the amino acid sequence of SEQ ID NO: 2.

In some other embodiments, at least one of the one or more light chains has an amino acid sequence containing a non-naturally encoded amino acid, wherein the amino acid sequence is selected from the group consisting of SEQ ID NO: 6, 7, 8 and 9.

In some embodiments, each [symbol] represents a double bond. In some embodiments, each double bond covalently joins one of the one or more drug-linker groups to one non-naturally encoded amino acid of the anti-CD70 antibody or fragment thereof. In some embodiments, each said non-naturally encoded amino acid is para-acetyl-L-phenylalanine.

Thus, in some embodiments, the double bond is between the drug-linker group and a non-naturally encoded para-acetyl-L-phenylalanine that has been incorporated into the antibody or fragment thereof. In some embodiments, the double bond is a product of a reaction between a terminal aminooxy group of a drug-linker compound and the acetyl group of the non-naturally encoded para-acetyl-L-phenylalanine incorporated into the anti-CD70 antibody or fragment thereof. Accordingly, in some embodiments, the drug-linker moiety is connected to the antibody or fragment thereof via an oxime group. In some embodiments, the drug-linker compound having a terminal aminooxy group is compound 6 having the following structure:

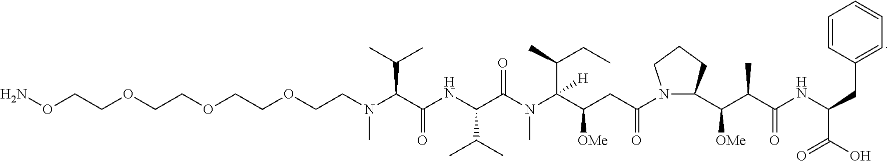 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)CCOCCOCCOCCON)C(C)C ; "."]

In some further embodiments, the ADC is an ADC of Formula (I):

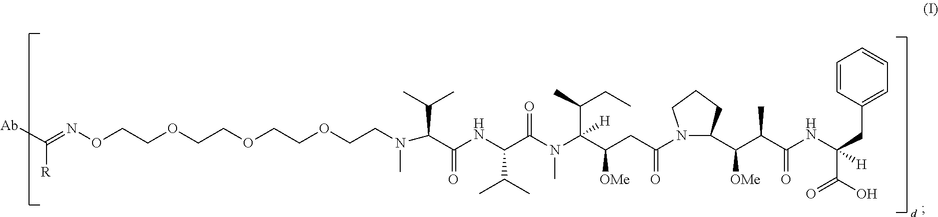

wherein:
Ab is the anti-CD70 antibody or fragment thereof;
each R is unsubstituted C₁-C₈ alkyl; and
d is at least 1 and is at most 10.

Thus, the ADC comprises at least 1 and at most 10 drug-linker groups, wherein each of the one or more drug-linker groups has the following structure:

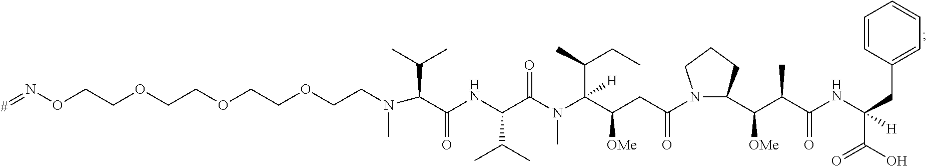

wherein each #represents a site of connection to the anti-CD70 antibody or fragment thereof.

In some embodiments, d is 1, 2, 3 or 4. Thus, in some embodiments, the ADC comprises 1, 2, 3 or 4 drug-linker groups, respectively. In some embodiments, d is at least 1 and is at most 2. Thus, in some embodiments, the ADC comprises at least 1 and at most 2 drug-linker groups.

In some embodiments, d is 2. Thus, the ADC comprises 2 drug-linker groups.

In some embodiments, each R is methyl. The R group can represent the methyl group of the acyl moiety of para-acetyl-L-phenylalanine that has been incorporated into the anti-CD70 antibody.

In some embodiments, the anti-CD70 antibody or fragment thereof is humanized.

In some embodiments, the anti-CD70 antibody or fragment thereof is a humanized monoclonal antibody comprising two heavy chains and two light chains, wherein each heavy chain has the amino acid sequence of SEQ ID NO: 3, and each light chain has the amino acid sequence of SEQ ID NO: 2. In some embodiments, the amino acid sequence of SEQ ID NO: 3 comprises one non-naturally-encoded amino acid, wherein the one non-naturally encoded amino acid is para-acetyl-L-phenylalanine. In some embodiments, d is 2.

In some other embodiments, the anti-CD70 antibody or fragment thereof is a humanized monoclonal antibody comprising two heavy chains and two light chains, wherein each heavy chain has the amino acid sequence of SEQ ID NO: 4, and each light chain has the amino acid sequence of SEQ ID NO: 2. In some embodiments, the amino acid sequence of SEQ ID NO: 4 comprises one non-naturally-encoded amino acid, wherein the one non-naturally encoded amino acid is para-acetyl-L-phenylalanine. In some embodiments, d is 2.

In some other embodiments, the anti-CD70 antibody or fragment thereof is a humanized monoclonal antibody comprising two heavy chains and two light chains, wherein each heavy chain has the amino acid sequence of SEQ ID NO: 5, and each light chain has the amino acid sequence of SEQ ID NO: 2. In some embodiments, the amino acid sequence of SEQ ID NO: 5 comprises one non-naturally-encoded amino acid, wherein the one non-naturally encoded amino acid is para-acetyl-L-phenylalanine. In some embodiments, d is 2.

In some embodiments, the anti-CD70 antibody or fragment thereof is a humanized anti-CD70 monoclonal antibody comprising two heavy chains, two light chains, and two non-naturally encoded amino acids, wherein:
each said heavy chain has the amino acid sequence of SEQ ID NO: 3, wherein SEQ ID NO: 3 comprises one non-naturally encoded amino acid, wherein each non-naturally encoded amino acid is para-acetyl-L-phenylalanine at position A114 (Kabat numbering) of SEQ ID NO: 3; and
each said light chain has the amino acid sequence of SEQ ID NO: 2;
each R is methyl;
the one or more drug-linker groups is two drug-linker groups; and
d is 2;
   wherein each said drug-linker group is connected to one of said para-acetyl-L-phenylalanine at position A114, thereby connecting each drug-linker group to the anti-CD70 monoclonal antibody.

In some embodiments, the ADC is the ADC of FIG. 1.

In some other embodiments, the anti-CD70 antibody or fragment thereof is a humanized anti-CD70 monoclonal antibody comprising two heavy chains, two light chains, and two non-naturally encoded amino acids, wherein:
each said heavy chain has the amino acid sequence of SEQ ID NO: 4, wherein SEQ ID NO: 4 comprises one non-naturally encoded amino acid, wherein each non-naturally encoded amino acid is para-acetyl-L-phenylalanine at position A136 (Kabat numbering) of SEQ ID NO: 4; and
each said light chain has the amino acid sequence of SEQ ID NO: 2;
each R is methyl;
the one or more drug-linker groups is two drug-linker groups; and
d is 2;
   wherein each said drug-linker group is connected to one of said para-acetyl-L-phenylalanine at position A136, thereby connecting each drug-linker group to the anti-CD70 monoclonal antibody.

In some other embodiments, the anti-CD70 antibody or fragment thereof is a humanized anti-CD70 monoclonal antibody comprising two heavy chains, two light chains, and two non-naturally encoded amino acids, wherein:
each said heavy chain has the amino acid sequence of SEQ ID NO: 5, wherein SEQ ID NO: 5 comprises one non-naturally encoded amino acid, wherein each non-naturally encoded amino acid is para-acetyl-L-phenylalanine at position L159 (Kabat numbering) of SEQ ID NO: 5; and
each said light chain has the amino acid sequence of SEQ ID NO: 2;
each R is methyl;
the one or more drug-linker groups is two drug-linker groups; and
d is 2;
   wherein each said drug-linker group is connected to one of said para-acetyl-L-phenylalanine at position L159, thereby connecting each drug-linker group to the anti-CD70 monoclonal antibody.

In some other embodiments, the anti-CD70 antibody or fragment thereof is a humanized anti-CD70 monoclonal antibody comprising two heavy chains and two light chains, wherein:
each said heavy chain has the amino acid sequence of SEQ ID NO: 3, wherein SEQ ID NO: 3 comprises one non-naturally encoded amino acid, wherein the non-naturally encoded amino acid is para-acetyl-L-phenylalanine at position A114 (Kabat numbering) of SEQ ID NO: 3; and
each said light chain has an amino acid sequence selected from the group consisting of:
   SEQ ID NO: 6, wherein SEQ ID NO: 6 comprises one non-naturally encoded amino acid, wherein the non-naturally encoded amino acid is para-acetyl-L-phenylalanine at position V110 of SEQ ID NO: 6;
   SEQ ID NO: 7, wherein SEQ ID NO: 7 comprises one non-naturally encoded amino acid, wherein the non-naturally encoded amino acid is para-acetyl-L-phenylalanine at position A112 of SEQ ID NO: 7;
   SEQ ID NO: 8, wherein SEQ ID NO: 8 comprises one non-naturally encoded amino acid, wherein the non-naturally encoded amino acid is para-acetyl-L-phenylalanine at position S114 of SEQ ID NO: 8; and
   SEQ ID NO: 9, wherein SEQ ID NO: 9 comprises one non-naturally encoded amino acid, wherein the non-naturally encoded amino acid is para-acetyl-L-phenylalanine at position S121 of SEQ ID NO: 9;
each R is methyl;
the one or more drug-linker groups is four drug-linker groups; and
d is 4;
   wherein each said drug-linker group is connected to one said para-acetyl-L-phenylalanine, thereby connecting each drug-linker group to the anti-CD70 monoclonal antibody.

In some other embodiments, the anti-CD70 antibody or fragment thereof is a humanized anti-CD70 monoclonal antibody comprising two heavy chains and two light chains, wherein:
each said heavy chain has the amino acid sequence of SEQ ID NO: 4, wherein SEQ ID NO: 4 comprises one non-naturally encoded amino acid, wherein the non-naturally encoded amino acid is para-acetyl-L-phenylalanine at position A136 (Kabat numbering) of SEQ ID NO: 4; and
each said light chain has an amino acid sequence selected from the group consisting of:
   SEQ ID NO: 6, wherein SEQ ID NO: 6 comprises one non-naturally encoded amino acid, wherein the non-naturally encoded amino acid is para-acetyl-L-phenylalanine at position V110 of SEQ ID NO: 6;
   SEQ ID NO: 7, wherein SEQ ID NO: 7 comprises one non-naturally encoded amino acid, wherein the non-naturally encoded amino acid is para-acetyl-L-phenylalanine at position A112 of SEQ ID NO: 7;
   SEQ ID NO: 8, wherein SEQ ID NO: 8 comprises one non-naturally encoded amino acid, wherein the non-naturally encoded amino acid is para-acetyl-L-phenylalanine at position S114 of SEQ ID NO: 8; and
   SEQ ID NO: 9, wherein SEQ ID NO: 9 comprises one non-naturally encoded amino acid, wherein the non-naturally encoded amino acid is para-acetyl-L-phenylalanine at position S121 of SEQ ID NO: 9;
each R is methyl;
the one or more drug-linker groups is four drug-linker groups; and
d is 4;
   wherein each said drug-linker group is connected to one said para-acetyl-L-phenylalanine, thereby connecting each drug-linker group to the anti-CD70 monoclonal antibody.

In some other embodiments, the anti-CD70 antibody or fragment thereof is a humanized anti-CD70 monoclonal antibody comprising two heavy chains and two light chains, wherein:
each said heavy chain has the amino acid sequence of SEQ ID NO: 5, wherein SEQ ID NO: 5 comprises one non-naturally encoded amino acid, wherein the non-naturally encoded amino acid is para-acetyl-L-phenylalanine at position L159 (Kabat numbering) of SEQ ID NO: 5; and
each said light chain has an amino acid sequence selected from the group consisting of:
   SEQ ID NO: 6, wherein SEQ ID NO: 6 comprises one non-naturally encoded amino acid, wherein the non-naturally encoded amino acid is para-acetyl-L-phenylalanine at position V110 of SEQ ID NO: 6;
   SEQ ID NO: 7, wherein SEQ ID NO: 7 comprises one non-naturally encoded amino acid, wherein the non-naturally encoded amino acid is para-acetyl-L-phenylalanine at position A112 of SEQ ID NO: 7;
   SEQ ID NO: 8, wherein SEQ ID NO: 8 comprises one non-naturally encoded amino acid, wherein the non-naturally encoded amino acid is para-acetyl-L-phenylalanine at position S114 of SEQ ID NO: 8; and
   SEQ ID NO: 9, wherein SEQ ID NO: 9 comprises one non-naturally encoded amino acid, wherein the non-naturally encoded amino acid is para-acetyl-L-phenylalanine at position S121 of SEQ ID NO: 9;
each R is methyl;
the one or more drug-linker groups is four drug-linker groups; and
d is 4;
   wherein each said drug-linker group is connected to one said para-acetyl-L-phenylalanine, thereby connecting each drug-linker group to the anti-CD70 monoclonal antibody.

It is understood that an ADC is typically produced as a composition containing a population of ADCs, i.e., a mixture of ADCs that are essentially identical, except for the drug load. As disclosed herein, an ADC composition can be characterized by a drug-to-antibody ratio (DAR), which reports on the average number of drugs conjugated to antibody in the ADC composition. Thus, in some aspects, the present disclosure provides an ADC composition comprising a mixture of ADCs, wherein each ADC in the mixture is identical, except that the number of drugs or drug-linkers that are conjugated to each antibody can vary.

In a non-limiting example, an ADC of the present disclosure comprises a first ADC, a second ADC, a third ADC and a fourth ADC, wherein the first ADC, the second ADC, the third ADC and the fourth ADC are identical, except that the first ADC comprises one drug or drug-linker, the second ADC comprises two drugs or drug-linkers, the third ADC comprises three drugs or drug-linkers, and the fourth ADC comprises four drugs or drug-linkers.

In another non-limiting example, there is provided an ADC composition, comprising:
(a) an ADC of Formula (I), wherein d is 1;
(b) an ADC of Formula (I), wherein the ADC is identical to (a), except that d is 2;
(c) an ADC of Formula (I), wherein the ADC is identical to (a), except that d is 3;
(d) an ADC of Formula (I), wherein the ADC is identical to (a), except that d is 4;
or a combination of any two or more of the foregoing; wherein the composition is characterized as having a DAR of at least about 1 and at most about 4.

In some embodiments, an ADC composition of the present disclosure is characterized as having a DAR of at least about 1 and at most about 8. In some embodiments, the ADC composition is characterized as having a DAR of at least about 1 and at most about 4. In some embodiments, the ADC composition is characterized as having a DAR of at least about 1 and at most about 2. In some embodiments, the ADC composition is characterized as having a DAR of about 2. In some other embodiments, the ADC composition is characterized as having a DAR of about 3. In yet some other embodiments, the ADC composition is characterized as having a DAR of about 4.

### Methodology and Techniques

The present disclosure encompasses methodologies and technologies well known in the art. These include conventional methods of mass spectroscopy, NMR, HPLC, protein chemistry, biochemistry, recombinant DNA techniques and pharmacology, within the skill of the art. Compounds of the present disclosure can be synthesized using several processes or schemes employed in the art. See for example, Dubowchik et al., Bioconjugate Chem. 13: 855-869, 2002; Doronina et al., Nature Biotechnology 21(7): 778-784, 2003; WO2012/166560; WO2013/185117, each incorporated herein by reference. Many methodologies and techniques for synthesis of pharmaceutical, diagnostic or therapeutic compounds are well known to one of ordinary skill in the art.

The present disclosure, unless otherwise indicated, also encompass conventional techniques of molecular biology (including recombinant techniques), cell biology, biochemistry and immunology, all within the skill of the art. Such techniques are explained fully in the literature, such as, Molecular Cloning: A Laboratory Manual, Third Edition, Cold Spring Harbor Press, Cold Spring Harbor, NY (Sambrook et al. Eds., 2001); Oligonucleotide Synthesis: Methods And Applications (Methods in Molecular Biology), Herdewijn, P., Ed., Humana Press, Totowa, NJ; Oligonucleotide Synthesis (Gait, M. J., Ed., 1984); Methods In Molecular Biology, Humana Press, Totowa, NJ; Cell Biology: A Laboratory Notebook, Academic Press, New York, NY (Cellis, J. E., Ed., 1998); Animal Cell Culture (Freshney, R. I., Ed., 1987); Introduction To Cell And Tissue Culture Plenum Press, New York, NY, (Mather, J. P. and Roberts, P. E., Eds., 1998); Cell And Tissue Culture: Laboratory Procedures John Wiley and Sons, Hoboken, NJ, (Doyle, A. et al., Eds., 1993-8); Methods In Enzymology (Academic Press, Inc.) New York, NY; Weir's Handbook Of Experimental Immunology Wiley-Blackwell Publishers, New York, NY, (Herzenberg, L. A. et al. Eds., 1997); Gene Transfer Vectors For Mammalian Cells Cold Spring Harbor Press, Cold Spring Harbor, NY, (Miller, J. M. et al. Eds., 1987); Current Protocols In Molecular Biology, Greene Pub. Associates, New York, NY, (Ausubel, F. M. et al., Eds., 1987); PCR: The Polymerase Chain Reaction, Birkhauser, Boston, MA, (Mullis, K. et al., Eds., 1994); Current Protocols In Immunology, John Wiley and Sons, Hoboken, NJ, (Coligan, J. E. et al., eds., 1991); Short Protocols In Molecular Biology, Hoboken, NJ, (John Wiley and Sons, 1999); Immunobiology 7 Garland Science, London, UK, (Janeway, C. A. et al., 2007); Antibodies. Stride Publications, Devoran, UK, (P. Finch, 1997); Antibodies: A Practical Approach Oxford University Press, USA, New York, NY, (D. Catty., ed., 1989); Monoclonal Antibodies: A Practical Approach Oxford University Press, USA, New York NY, (Shepherd, P. et al. Eds., 2000); Using Antibodies: A Laboratory Manual Cold Spring Harbor Laboratory Press, Cold Spring Harbor, NY, (Harlow, E. et al. Eds., 1998); The Antibodies Harwood Academic Publishers, London, UK, (Zanetti, M. et al. Eds. 1995).

### Therapeutic Uses of ADCs

The antibodies or ADCs of the disclosure are useful for treating a wide range of diseases, disorders, conditions or cancers. The disclosure includes compositions for use in, and methods of treating, a mammal that is at risk for, is having, and/or has had a disease or condition, such as a cancer, that is responsive to CD70 overexpression, amplification, mutation and/or targeted therapies. Compositions disclosed herein may be used to modulate an immune response. Modulation of an immune response may comprise stimulating, activating, increasing, enhancing, or up-regulating an immune response. Modulation of an immune response may comprise suppressing, inhibiting, preventing, reducing, or downregulating an immune response. In some embodiments, the ADCs of the present invention may be used for reducing or inhibiting tumor growth or progression in an antigen-expressing cancer or cancer cell comprising an effective amount of the ADC.

Administration of ADCs may result in a short-term effect, i.e., an immediate beneficial effect on several clinical parameters observed and this may occur 12 or 24 hours from the time of administration, and/or may result in a long-term effect, such as a beneficial slowing of progression of tumor growth or reduction in tumor size. The ADC of the present disclosure may be administered by any means known to those skilled in the art, and may beneficially be administered via infusion, e.g., by arterial, intraperitoneal or intravenous injection and/or infusion in a dosage which is sufficient to obtain the desired pharmacological effect. In some embodiments, the ADC, or the composition or formulation comprising the ADC, is administered orally, intradermally, intratumorally, intravenously or subcutaneously. In some embodiments, the ADC, or the composition or formulation comprising the ADC, is administered intravenously.

Disclosed herein are methods of treating a subject for a disease or condition with an ADC or pharmaceutical composition of the disclosure. The invention provides a method of treating a disease or condition, such as a tumor or cancer, by administering to a patient (e.g., a human subject) in need thereof a therapeutically effective amount of an ADC of the present disclosure, or a pharmaceutical composition comprising an ADC of the present disclosure.

The dose administered to a patient in the context of the present disclosure can be sufficient to cause a beneficial response in the subject over time. The ADC dosage may be given as a bolus injection and/or as an infusion for a clinically necessary period of time, e.g., for a period ranging from a few minutes to several hours, e.g., up to 24 hours. If necessary, the ADC administration may be repeated one or several times. The ADC dosage may be an effective amount or dose.

In some embodiments, the effective amount of ADC is a dose of at least about 0.05 mg/kg of the human subject. In some embodiments, the effective amount of ADC is a dose of at least about 0.1 mg/kg of the human subject. In some embodiments, the effective amount of ADC is a dose of at least about 0.15 mg/kg of the human subject. In some embodiments, the effective amount of ADC is a dose of at least about 0.2 mg/kg of the human subject.

In some embodiments, the effective amount of ADC is a dose within a range of about 0.05 mg/kg to about 10 mg/kg of the human subject.

In some embodiments, the effective amount of ADC is a dose within a range of about 0.05 mg/kg to 2 mg/kg of the human subject or any value in between. In some embodiments, the effective amount of ADC is a dose within a range of about 0.05 mg/kg to 1.9 mg/kg of the human subject or any value in between. In some embodiments, the effective amount of ADC is a dose within a range of about 0.05 mg/kg to 1.8 mg/kg of the human subject or any value in between. In some embodiments, the effective amount of ADC is a dose within a range of about 0.05 mg/kg to 1.7 mg/kg of the human subject or any value in between. In some embodiments, the effective amount of ADC is a dose within a range of about 0.05 mg/kg to 1.6 mg/kg of the human subject or any value in between. In some embodiments, the effective amount of ADC is a dose within a range of about 0.05 mg/kg to 1.5 mg/kg of the human subject or any value in between. In some other embodiments, the effective amount of the ADC is a dose within a range of about 0.1 mg/kg to 2 mg/kg, 0.1 mg/kg to 1.9 mg/kg, 0.1 mg/kg to 1.8 mg/kg, 0.1 mg/kg to 1.7 mg/kg, 0.1 mg/kg to 1.6 mg/kg or 0.1 mg/kg to 1.5 mg/kg of the human subject. In some other embodiments, the effective amount of ADC is a dose within a range of about 0.2 mg/kg to 2 mg/kg, 0.2 mg/kg to 1.9 mg/kg, 0.2 mg/kg to 1.8 mg/kg, 0.2 mg/kg to 1.7 mg/kg, 0.2 mg/kg to 1.6 mg/kg or 0.2 mg/kg to 1.5 mg/kg of the human subject.

In some embodiments, the effective amount of the ADC is a dose of about 0.05 mg/kg, about 0.1 mg/kg, about 0.12 mg/kg, about 0.14 mg/kg, about 0.16 mg/kg, about 0.18 mg/kg, about 0.2 mg/kg, about 0.22 mg/kg, about 0.24 mg/kg, about 0.26 mg/kg, about 0.28 mg/kg, about 0.3 mg/kg, about 0.32 mg/kg, about 0.34 mg/kg, about 0.36 mg/kg, about 0.38 mg/kg, about 0.4 mg/kg, about 0.42 mg/kg, about 0.44 mg/kg, about 0.46 mg/kg, about 0.48 mg/kg, about 0.5 mg/kg, about 0.52 mg/kg, about 0.54 mg/kg, about 0.56 mg/kg, about 0.58 mg/kg, about 0.6 mg/kg, about 0.62 mg/kg, about 0.64 mg/kg, about 0.66 mg/kg, about 0.68 mg/kg, about 0.7 mg/kg, about 0.72 mg/kg, about 0.74 mg/kg, about 0.76 mg/kg, about 0.78 mg/kg, about 0.8 mg/kg, about 0.82 mg/kg, about 0.84 mg/kg, about 0.86 mg/kg, about 0.88 mg/kg, about 0.9 mg/kg, about 0.92 mg/kg, about 0.94 mg/kg, about 0.96 mg/kg, about 0.98 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg or about 2 mg/kg of the human subject.

In some other embodiments, the effective amount of the ADC is a dose within a range of about 2 mg/kg to about 5 mg/kg of the human subject, or any value in between. In some embodiments, the effective amount of the ADC is a dose of about 2 mg/kg, about 2.2 mg/kg, about 2.4 mg/kg, about 2.6 mg/kg, about 2.8 mg/kg, about 3 mg/kg, about 3.2 mg/kg, about 3.4 mg/kg, about 3.6 mg/kg, about 3.8 mg/kg, about 4 mg/kg, about 4.2 mg/kg, about 4.4 mg/kg, about 4.6 mg/kg, about 4.8 mg/kg or about 5 mg/kg of the human subject.

In some other embodiments, the effective amount of the ADC is a dose within a range of about 5 mg/kg to about 10 mg/kg of the human subject, or any value in between. In some embodiments, the effective amount of an ADC is a dose of about 5 mg/kg, about 5.2 mg/kg, about 5.4 mg/kg, about 5.6 mg/kg, about 5.8 mg/kg, about 6 mg/kg, about 6.2 mg/kg, about 6.4 mg/kg, about 6.6 mg/kg, about 6.8 mg/kg, about 7 mg/kg, about 7.2 mg/kg, about 7.4 mg/kg, about 7.6 mg/kg, about 7.8 mg/kg, about 8 mg/kg, about 8.2 mg/kg, about 8.4 mg/kg, about 8.6 mg/kg, about 8.8 mg/kg, about 9 mg/kg, about 9.2 mg/kg, about 9.4 mg/kg, about 9.6 mg/kg, about 9.8 mg/kg or about 10 mg/kg of the human subject.

Average quantities of the ADC may vary and in particular can be based upon the recommendations and prescription of a qualified physician. The exact amount of ADC is a matter of preference subject to such factors as the exact type of condition being treated, the condition of the patient being treated, as well as the other ingredients in the composition. The disclosure also provides for administration of a therapeutically effective amount of another active agent. The amount to be given may be readily determined by one of ordinary skill in the art based upon therapy with ADCs.

A tumor or cancer to be treated by an ADC of the present invention can be a solid tumor or a hematologic tumor or cancer. In some embodiments, the tumor or cancer is a hematologic cancer, such as lymphoma, multiple myeloma or leukemia. In some embodiments, the tumor or cancer is kidney cancer, brain cancer, breast cancer, Burkitt's lymphoma, ovarian cancer, gastric cancer, gastro-esophageal junction adenocarcinoma, cervical cancer, uterine cancer, endometrial cancer, testicular cancer, prostate cancer, colorectal cancer, esophageal cancer, bladder cancer, lung cancer, non-small cell lung cancer (NSCLC), urothelial carcinoma, cholangiocarcinoma, colon biliary tract cancer, pancreatic cancer, renal cell cancer, nasopharyngeal cancer, mantle cell lymphoma, multiple myeloma, non-Hodgkin's lymphoma, Hodgkin's lymphoma or acute myeloid leukemia, or a cancer or disease or conditions related to any of these cancers. In some embodiments, the tumor or cancer is renal cell cancer, brain cancer, multiple myeloma, mantle cell lymphoma or lung cancer. In some embodiments, the tumor or cancer is renal cell cancer (RCC). In some embodiments, the tumor or cancer is clear cell renal cell cancer (ccRCC). In some other embodiments, the tumor or cancer is brain cancer. In some embodiments, the tumor or cancer is multiple myeloma. In some embodiments, the tumor or cancer is mantle cell lymphoma. In some embodiments, the tumor or cancer is lung cancer.

In some embodiments, the tumor or cancer is renal cell cancer (RCC). In some embodiments, the renal cell cancer is metastatic renal cell cancer. In some embodiments, the renal cell cancer is clear cell renal cell carcinoma. In some embodiments, the tumor or cancer is an MDR-positive renal cell carcinoma. In some embodiments, the tumor or cancer is an MDR-positive clear cell renal cell carcinoma. In some embodiments, the patient receiving the treatment is resistant or refractory to prior standard therapies. In some embodiments, the patient is an adult.

In some embodiments, the tumor or cancer to be treated is a CD70-expressing cancer. Thus, in some embodiments, ADCs of the present disclosure can be used for treating cancer in a cell expressing high CD70 surface number. In some embodiments, the tumor or cancer to be treated is a CD70-expressing cell having at least about 10,000 CD70/cell, or at least about 15,000 CD70/cell. The cancer may be treated by recruiting cytotoxic T cells to the antigen receptor-expressing tumor cells. In some embodiments, the disclosure provides a method of treating any cancer, disease or condition associated with high expression of antigen receptors by administering to a patient a therapeutically-effective amount of an antibody or ADC of the disclosure. In some embodiments, the antibody or antibody fragment of the ADC binds to a tumor-associated CD70 antigen.

In some embodiments, the patient to be treated has a CD70 expressing cancer and/or cancer metastases from a same or different cancer.

In some embodiments, the method of treatment improves or optimizes cancer cell kill. In some embodiments, the method delays progression or recurrence of the tumor or cancer.

In some aspects, the antibodies or ADCs of the present invention disclosure can be used in conjunction with an additional therapy or treatment, including but not limited to surgery, radiation, cryosurgery, thermotherapy, hormone treatment, chemotherapy, vaccines and other immunotherapies.

Thus, in some embodiments, the present disclosure provides a method of treating a disease or condition, such as a tumor or cancer, wherein the method comprises administering to a subject an effective amount of an ADC of the present disclosure and an additional therapeutic agent and/or radiation therapy (radiotherapy). In some embodiments, the additional therapeutic agent is a chemotherapeutic agent, a hormonal agent, an antitumor agent, an immunostimulatory agent, an immunomodulator or an immunotherapeutic agent, or a combination thereof. In some other embodiments, the additional therapeutic agent is a checkpoint inhibitor, a CD70 kinase inhibitor, cyclin-dependent kinase inhibitor, tyrosine kinase inhibitor, small-molecule kinase inhibitor, hypomethylating agent or a platinum-based therapeutic, or a combination thereof. In some other embodiments, the therapeutic agent is a CD70 targeted therapeutic.

In some aspects, a subject is treated with an anti-CD70 antibody or ADC of the present disclosure and an additional agent, wherein the additional agent is an immune checkpoint inhibitor. Immune checkpoint inhibitors (or simply “checkpoint inhibitors” herein) can function as tumor suppressors by modulating the interaction between immune cells and tumor cells (see, e.g., Alsaab, H. O. et al., Frontiers in Pharmacology, Vol. 8, Article 561 (2017); https://doi.org/10.3389/fphar.2017.00561). In some embodiments, the checkpoint inhibitor is a CTLA-4 inhibitor, which targets or inhibits cytotoxic T-cell lymphocyte-associated protein 4 (CTLA-4). In some embodiments, the CTLA-4 inhibitor is ipilimumab. In some other embodiments, the checkpoint inhibitor is a PD-1 inhibitor, which targets or inhibits programmed death receptor 1 (PD-1). In some other embodiments, the checkpoint inhibitor is a PD-L1 inhibitor, which targets or inhibits PD-1 ligand 1 (PD-L1). In some embodiments, the checkpoint inhibitor targets or inhibits PD-1 and/or PD-L1; said checkpoint inhibitor may be referred to herein as a “PD-1/PD-L1 inhibitor.” Non-limiting examples of PD-1/PD-L1 inhibitors include AMP-224, atezolizumab, avelumab, BMS-936558, BMS-936559, CT-001, durvalumab, MEDI0680, nivolumab, PDR001, pembrolizumab, pidilizumab and REGN2810. Thus, in some embodiments, the present disclosure provides a method of treating cancer in a subject, wherein the method comprises administering an ADC of the present disclosure and an additional therapeutic agent to the subject, wherein the additional therapeutic agent is a checkpoint inhibitor. In some embodiments, the checkpoint inhibitor is a PD-1/PD-L1 inhibitor or a CTLA-4 inhibitor. In some embodiments, the checkpoint inhibitor is selected from the group consisting of AMP-224, atezolizumab, avelumab, BMS-936558, BMS-936559, CT-001, durvalumab, ipilimumab, MEDI0680, nivolumab, PDR001, pembrolizumab, pidilizumab and REGN2810; and combinations thereof.

As disclosed herein, treatments with some therapeutic agents can upregulate CD70. The present disclosure provides for use of such therapeutic agents in combination with an anti-CD70 antibody or ADC of the present disclosure.

In some aspects, a subject is treated with an anti-CD70 antibody or ADC of the present disclosure and an additional agent, wherein the additional agent is a hypomethylating agent. Hypomethylating agents, also known as demethylating agents, are chemotherapeutic agents that have been shown to upregulate CD70 in conditions including acute myeloid leukemia (AML) and myelodysplastic syndrome (MDS) (see, e.g., Stomper, J. et al., Leukemia 35:1873-1889 (2021)). Non-limiting examples of hypomethylating agents include azacitidine (5-azacytidine), cytidine, decitabine (5-aza-2′-deoxycytidine) and guadecitabine. Thus, in some embodiments, there is provided a method of treating a disease or condition in a subject, wherein the method comprises administering an anti-CD70 ADC of the present disclosure and an additional therapeutic agent to the subject, wherein the additional therapeutic agent is a hypomethylating agent. In some embodiments, the hypomethylating agent is selected from the group consisting of azacytidine, cytidine, decitabine and guadecitabine. In some embodiments, the disease or condition is cancer. In some embodiments, the cancer is AML. In some other embodiments, the cancer is renal cell carcinoma (RCC). In some embodiments, the RCC is metastatic RCC. In some embodiments, the RCC is clear cell RCC. In some other embodiments, the disease or condition is MDS.

In some aspects, a subject is treated with an anti-CD70 antibody or ADC of the present disclosure and an additional agent, wherein the additional agent is a platinum-based chemotherapeutic agent. Non-limiting examples of platinum-based chemotherapeutic agents include cisplatin, oxaliplatin and carboplatin. Cisplatin reportedly upregulates CD70 in cancer, including non-small cell lung cancer (NSCLC; see, e.g., Flieswasster, T. et al., J Exp Clin Cancer Res. 41: 12 (2022)). Thus, in some embodiments, the present disclosure provides a method of treating cancer in a subject, wherein the method comprises administering an ADC of the present disclosure and an additional therapeutic agent to the subject, wherein the additional therapeutic agent is a platinum-based therapeutic. In some embodiments, the platinum-based therapeutic is cisplatin, oxaliplatin or carboplatin. In some embodiments, the platinum-based therapeutic is cisplatin. In some embodiments, the cancer is NSCLC. In some other embodiments, the cancer is renal cell carcinoma (RCC). In some embodiments, the RCC is metastatic RCC. In some embodiments, the RCC is clear cell RCC.

In some aspects, a subject is treated with an anti-CD70 antibody or ADC of the present disclosure and an additional agent, wherein the additional agent is a tyrosine kinase inhibitor (TKI). Non-limiting examples of TKIs include axitinib, cabozantinib, dasatinib, everolimus, erlotinib, gefitinib, imatinib, lapatinib, lenvatinib, pazopanib, and sunitinib. TKIs have been reported to upregulate CD70 in chronic myelogenous leukemia (CML; see Riether, C. et al., https://doi.org/10.7892/boris.77241). TKIs are also used to treat metastatic renal cell carcinoma (RCC) patients (see Stitt, T. M. et al., Journal of Hematology Oncology Pharmacy, 12(3):138-144 (2022)). Cancers characterized by epidermal growth factor receptor (EGFR) mutations can acquire resistance to EGFR TKIs. Epithelial-mesenchymal transition (EMT) has been associated with acquired EGFR-TKI resistance in NSCLC, and CD70 is highly upregulated in EMT-associated resistance. Anti-CD70 ADCs show potent activity against EGFR TKI-resistant cells, suggesting that CD70 is a suitable therapeutic target for EGFR mutant tumors with acquired EGFR TKI resistance (see, e.g., Nilsson, M. B. et al., Cancer Cell, 41(2):340-355 (2023)). Thus, in some embodiments, the present disclosure provides a method of treating cancer in a subject, wherein the method comprises administering an ADC of the present disclosure and an additional therapeutic agent to the subject, wherein the additional therapeutic agent is a tyrosine kinase inhibitor. In some embodiments, the tyrosine kinase inhibitor is selected from the group consisting of axitinib, cabozantinib, dasatinib, everolimus, erlotinib, gefitinib, imatinib, lapatinib, lenvatinib, pazopanib and sunitinib; and combinations thereof. In some embodiments, the cancer is an EGFR mutant tumor with acquired EGFR TKI resistance. In some embodiments, the cancer is lung cancer. In some embodiments, the lung cancer is NSCLC. In some other embodiments, the cancer is CML. In some other embodiments, the cancer is renal cell carcinoma (RCC). In some embodiments, the RCC is metastatic RCC. In some embodiments, the RCC is clear cell RCC.

In some aspects, a subject is treated with an anti-CD70 antibody or ADC of the present disclosure and an additional agent, wherein the additional agent is radiation. Radiation therapy (radiotherapy) can also upregulate CD70 expression, for example, in glioma, leukemia and lymphoma (Flieswasster, T. et al., J Exp Clin Cancer Res. 41: 12 (2022)). Thus, in some embodiments, the present disclosure provides a method of treating cancer in a subject, wherein the method comprises administering an ADC of the present disclosure to the subject in combination with radiation therapy. In some embodiments, the cancer is glioma, leukemia or lymphoma. In some other embodiments, the cancer is renal cell carcinoma (RCC). In some embodiments, the RCC is metastatic RCC. In some embodiments, the RCC is clear cell RCC.

It is understood that the antibodies, compounds, ADCs or compositions of the disclosure can be used in the manufacture of a medicament for treating a disease or condition including cancer.

A pharmaceutical composition containing an antibody or ADC of the invention can be formulated at a strength effective for administration by various means to a human patient experiencing disorders that may be affected by antibody agonists or antagonists, such as but not limited to, anti-proliferatives, anti-inflammatory, or anti-virals are used, either alone or as part of a condition or disease. Average quantities of an antibody or ADC may vary and in particular should be based upon the recommendations and prescription of a qualified physician. The exact amount of an antibody or ADC is a matter of preference subject to such factors as the exact type of condition being treated, the condition of the patient being treated, as well as the other ingredients in the composition. The disclosure also provides for administration of a therapeutically effective amount of another active agent such as an anti-cancer chemotherapeutic agent or immunotherapeutic agent but is not limited to such. The amount to be given may be readily determined by one skilled in the art based upon therapy with the antibody or ADCs of the invention.

### CD70-Associated Disorders

As disclosed herein, the anti-CD70 antibodies and ADCs described herein are useful for treating or preventing a CD70-expressing cancer or an immunological disorder characterized by expression of CD70 by inappropriate activation of immune cells (e.g., lymphocytes or dendritic cells). Such expression of CD70 can be due to, for example, increased CD70 protein levels on the cells surface and/or altered antigenicity of the expressed CD70. Treatment or prevention of the immunological disorder, according to the methods described herein, is achieved by administering to a subject in need of such treatment or prevention an effective amount of the anti-CD70 antibody or derivative, whereby the antibody or derivative (i) binds to activated immune cells that express CD70 and that are associated with the disease state and (ii) exerts a cytotoxic, cytostatic, or immunomodulatory effect on the activated immune cells. In some embodiments, the cytotoxic, cytostatic, or immunomodulatory is exerted without conjugation to a cytotoxic, cytostatic, or immunomodulatory agent. In some embodiments, the cytotoxic, cytostatic, or immunomodulatory is exerted by conjugation to a cytotoxic, cytostatic, or immunomodulatory agent.

The anti-CD70 antibodies and ADCs described herein are also useful for treating or preventing a CD70-expressing cancer. Treatment or prevention of a CD70-expressing cancer, according to the methods described herein, is achieved by administering to a subject in need of such treatment or prevention an effective amount of the anti-CD70 antibody or derivative or ADC, whereby the antibody or derivative or ADC (i) binds to CD70-expressing cancer cells and (ii) exerts a cytotoxic or cytostatic effect to deplete or inhibit the proliferation of the CD70-expressing cancer cells. In some embodiments, the cytotoxic, cytostatic, or immunomodulatory effect is exerted without conjugation to a cytotoxic, cytostatic, or immunomodulatory agent. In some embodiments, the cytotoxic, cytostatic, or immunomodulatory effect is exerted by conjugation to a cytotoxic, cytostatic, or immunomodulatory agent. In some embodiments, the cyotoxic, cytostatic or immunomodulatory effect is exerted by an anti-CD70 ADC comprising anti-CD70 antibody conjugated to drug-linker AS269, i.e., an anti-CD70-AS269 ADC of the present disclosure.

CD70-expressing cancers that can be treated or prevented by the methods described herein include, for example, different subtypes of Non-Hodgkin's Lymphoma (indolent NHLs, follicular NHLs, small lymphocytic lymphomas, lymphoplasmacytic NHLs, or marginal zone NHLs); Hodgkin's disease (Hodgkin's lymphoma; e.g., Reed-Sternberg cells); cancers of the B-cell lineage, including, e.g., diffuse large B-cell lymphomas, follicular lymphomas, Burkitt's lymphoma, mantle cell lymphomas, B-cell lymphocytic leukemias (e.g., acute lymphocytic leukemia, chronic lymphocytic leukemia); Epstein Barr Virus positive B cell lymphomas; renal cell carcinomas (e.g., clear cell renal cell carcinoma, papillary renal cell carcinoma); nasopharyngeal carcinomas; thymic carcinomas; brain cancer; gliomas; glioblastomas; neuroblastomas; astrocytomas; meningiomas; Waldenstrom macroglobulinemia; multiple myelomas; lung cancers; pancreatic cancers; and colon, stomach, and rectal carcinomas. The cancer can be, for example, newly diagnosed, pre-treated or refractory or relapsed. In some embodiments, a CD70-expressing cancer has at least about 15,000, at least about 10,000 or at least about 5,000 CD70 molecules/cell. In some embodiments, the cancer or tumor can be a multi-drug resistant CD70-expressing cancer or tumor. In some embodiments, the cancer is a multi-drug resistant (MDR) positive renal cell carcinoma. In some more particular embodiments, the cancer is an MDR-positive clear cell renal cell carcinoma.

In some other embodiments, the disease or condition to be treated is myelodysplastic syndrome. In some embodiments, the subject has previously been treated with, or is undergoing treatment with, a hypomethylating agent.

### Pharmaceutical Compositions

In other aspects, there are provided pharmaceutical compositions or formulations containing the antibody, antibody fragments, variants or ADCs of the present disclosure. Such pharmaceutical compositions can employ various pharmaceutically acceptable excipients, stabilizers, buffers, and other components for administration to animals. See, for example, Remington, The Science and Practice of Pharmacy, 19th ed., Gennaro, ed., Mack Publishing Co., Easton, PA, 1995. Identifying suitable composition or formulations for stability, administration to a subject, and activity varies with each compound as a number of components, (for example, purifying, stabilizing components), need to be considered. Suitable salts for inclusion into the composition or formulation can include, but not limited to, sodium chloride, potassium chloride or calcium chloride. Buffering and/or stabilizing agents such as sodium acetate can be used. Suitable buffers can include phosphate-citrate buffer, phosphate buffer, citrate buffer, histidine buffer, L-histidine, L-arginine hydrochloride, bicarbonate buffer, succinate buffer, citrate buffer, and TRIS buffer, either alone or in combination. Surfactants can also be employed, including polysorbates (e.g., polysorbate 80), dodecyl sulfate (SDS), lecithin either alone or in combination.

In some embodiments, a pharmaceutical composition can be a formulation, comprising an ADC of the present disclosure and one or more pharmaceutically acceptable excipients, stabilizers or buffers.

In some embodiments, the formulation can contain the ADC and a buffer, cryoprotectant or surfactant; or any combination thereof.

In some embodiments, the formulation can contain the ADC at a concentration within a range of about 5 mg/mL to about 25 mg/mL. In some embodiments, the formulation can contain the ADC at a concentration of about 5 mg/mL, about 10 mg/mL, about 15 mg/mL, about 20 mg/mL or about 25 mg/mL. In some embodiments, the formulation can contain the ADC at a concentration within a range of about 5 mg/mL to about 15 mg/mL. In some embodiments, the formulation contains the ADC at a concentration of about 5 mg/mL. In some embodiments, the formulation contains the ADC at a concentration of about 10 mg/mL. In some embodiments, the formulation contains the ADC at a concentration of about 15 mg/mL.

In some embodiments, the formulation can contain a buffer. In some embodiments, the buffer is acetate buffer, succinate buffer, histidine buffer or phosphate buffer. In some embodiments, the buffer is a histidine buffer. In some embodiments, the formulation can have a histidine buffer concentration within a range of about 10 mM to about 50 mM. In some embodiments, the formulation can have a histidine buffer concentration of about 5 mM, about 10 mM, about 15 mM, about 20 mM, about 25 mM, about 30 mM, about 35 mM, about 40 mM, about 45 mM or about 50 mM. In some embodiments, the formulation can have a histidine buffer concentration within a range of about 5 mM to about 25 mM. In some embodiments, the formulation can have a histidine buffer concentration of about 5 mM, about 10 mM, about 15 mM, about 20 mM or about 25 mM. In some embodiments, the formulation can have a histidine buffer concentration within a range of about 15 mM to about 25 mM. In some embodiments, the formulation can have a histidine buffer concentration of about 15 mM. In some embodiments, the formulation can have a histidine buffer concentration of about 20 mM. In some embodiments, the formulation can have a histidine buffer concentration of about 25 mM. In some embodiments, the histidine is L-histidine. In some embodiments, the histidine buffer comprises L-histidine and L-histidine hydrochloride. Various combinations of L-histidine and L-histidine hydrochloride concentrations can be used by the person of ordinary skill in the art to achieve a target pH of the histidine buffer.

In some embodiments, the formulation is characterized as having a pH value. In some embodiments, the formulation can have a pH within a range of about 5 to about 7.4. In some embodiments, the formulation can have a pH of at most about 7, at most about 6.5 or at most about 6. In some embodiments, the formulation can have a pH within a range of about 5.4 to about 6.4. In some embodiments, the formulation can have a pH within a range of about 5.2 to about 6.2. In some embodiments, the formulation can have a pH of about 5.0, about 5.1, about 5.2, about 5.3, about 5.4, about 5.5, about 5.6, about 5.7, about 5.8, about 5.9, about 6.0, about 6.1, about 6.3, about 6.4 or about 6.5. In some embodiments, the formulation pH is less than 6. In some embodiments, the formulation pH is about 5.5. In some embodiments, the formulation pH is about 5.6. In some embodiments, the formulation pH is about 5.7. In some embodiments, the formulation pH is about 5.8. In some embodiments, the formulation pH is about 5.9.

In some embodiments, the formulation contains a cryoprotectant. In some embodiments, the cryoprotectant is polyvinyl pyrrolidone, glycerol, trehalose, fructose, sucrose, glucose or mannose; or a combination thereof. In some embodiments, the formulation has a cryoprotectant concentration within a range of about 1% (w/v) to about 20% (w/v). In some embodiments, the formulation has a cryoprotectant concentration of at most about 15% (w/v). In some other embodiments, the formulation has a cryoprotectant concentration within a range of about 5% (w/v) and about 15% (w/v). In some embodiments, the formulation has a cryoprotectant concentration of at most about 10% (w/v). In some embodiments, the formulation has a cryoprotectant concentration of about 1%, about 2%, about 3%, about 4%, about 5%, about 6%, about 7%, about 8%, about 9%, about 10%, about 11%, about 12%, about 13%, about 14% or about 15% (w/v). In some embodiments, the cryoprotectant is sucrose. In some embodiments, the formulation has a sucrose concentration within a range of about 1% (w/v) to about 20% (w/v). In some embodiments, the formulation has a sucrose concentration of at most about 15% (w/v). In some other embodiments, the formulation has a sucrose concentration within a range of about 5% (w/v) and about 15% (w/v). In some embodiments, the formulation has a sucrose concentration of at most about 10% (w/v). In some embodiments, the formulation has a sucrose concentration within a range of about 5% (w/v) and about 8% (w/v). In some embodiments, the formulation has a sucrose concentration of about 1%, about 2%, about 3%, about 4%, about 5%, about 6%, about 7%, about 8%, about 9%, about 10%, about 11%, about 12%, about 13%, about 14% or about 15% (w/v). In some embodiments, the formulation has a sucrose concentration of about 5% (w/v). In some embodiments, the formulation has a sucrose concentration of about 6% (w/v). In some embodiments, the formulation has a sucrose concentration of about 7% (w/v). In some embodiments, the formulation has a sucrose concentration of about 8% (w/v). In some embodiments, the formulation has a sucrose concentration of about 9% (w/v). In some embodiments, the formulation has a sucrose concentration of about 10% (w/v).

In some embodiments, the formulation can contain a surfactant. In some embodiments, the surfactant is a polysorbate. In some embodiments, the surfactant is polysorbate 20. In some other embodiments, the surfactant is polysorbate 80. In some embodiments the formulation has a surfactant concentration of at most about 1% (w/v). In some embodiments, the formulation has a surfactant concentration of at most about 0.1% (w/v). In some embodiments, the formulation has a surfactant concentration within a range of about 0.01% (w/v) to about 0.1% (w/v). In some embodiments, the formulation has a surfactant concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v) or about 0.10% (w/v). In some embodiments the formulation has a polysorbate 80 concentration of at most about 1% (w/v). In some embodiments, the formulation has a polysorbate 80 concentration of at most about 0.1% (w/v). In some embodiments, the formulation has a polysorbate 80 concentration within a range of about 0.01% (w/v) to about 0.1% (w/v). In some embodiments, the formulation has a polysorbate 80 concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v) or about 0.10% (w/v). In some embodiments, the formulation has a polysorbate 80 concentration within a range of about 0.01% (w/v) to about 0.05% (w/v). In some embodiments, the formulation has a polysorbate 80 concentration of about 0.01% (w/v). In some embodiments, the formulation has a polysorbate 80 concentration of about 0.02% (w/v). In some embodiments, the formulation has a polysorbate 80 concentration of about 0.03% (w/v). In some embodiments, the formulation has a polysorbate 80 concentration of about 0.04% (w/v). In some embodiments, the formulation has a polysorbate 80 concentration of about 0.05% (w/v).

In some embodiments, there is provided a formulation comprising an ADC of the present disclosure, histidine buffer, sucrose and polysorbate 80. In some embodiments, the formulation comprises an ADC (i.e., anti-CD70-AS269 ADC) at a concentration within a range of about 5 mg/mL to about 25 mg/mL; histidine buffer at a concentration within a range of about 10 mM to about 50 mM; sucrose at a concentration within a range of about 1% (w/v) to about 20% (w/v); and polysorbate 80 at a concentration within a range of about 0.01% (w/v) to about 0.1% (w/v). In some embodiments, the formulation has a pH within a range of about 5.4 to about 6.4. In some embodiments, the formulation has a pH within a range of about 5.2 to about 6.2. In some embodiments, the formulation pH is no greater than about 6. In some embodiments, the formulation pH is less than 6. In some further embodiments, the formulation comprises anti-CD70-AS269 ADC at a concentration within a range of about 5 mg/mL to about 15 mg/mL; histidine buffer at a concentration within a range of about 10 mM to about 25 mM; sucrose at a concentration within a range of about 5% (w/v) to about 15% (w/v); and polysorbate 80 at a concentration within a range of about 0.01% (w/v) to about 0.05% (w/v); wherein the formulation has a pH within a range of about 5.4 to about 6. In some embodiments, the formulation pH is no greater than about 6. In some embodiments, the formulation pH is less than 6. In some embodiments, the formulation contains the ADC at a concentration of about 10 mg/mL; histidine buffer at a concentration of about 20 mM; sucrose at a concentration of about 8% (w/v); and polysorbate 80 at a concentration of about 0.02% (w/v); wherein the formulation has a pH within a range of about 5.4 to about 6. In some embodiments, the formulation pH is no greater than about 6. In some embodiments, the formulation pH is less than 6. In some embodiments, the formulation pH is about 5.5. In some embodiments, the formulation pH is about 5.6. In some embodiments, the formulation pH is about 5.7. In some embodiments, the formulation pH is about 5.8. In some embodiments, the formulation pH is about 5.9.

In some embodiments, the formulation is a liquid formulation. In some embodiments, liquid formulation of the present disclosure can be stored at room temperature. In some other embodiments, the liquid formulation can be stored frozen.

A formulation of the present disclosure can be lyophilized to produce a lyophilized drug product containing an ADC or an antibody or fragment thereof of the present disclosure. The lyophilized drug product can be reconstituted with a suitable diluent, e.g., prior to administration. In some embodiments, the diluent is water, such as water-for-injections (WFI). Individual vials containing lyophilized drug product can contain ADC (e.g., anti-CD70-AS269 ADC) in an amount of, e.g., about 5 mg ADC, about 10 mg ADC, about 15 mg ADC, about 20 mg ADC, about 25 mg ADC, about 30 mg ADC, about 35 mg ADC, about 40 mg ADC, about 45 mg ADC, about 50 mg ADC, about 55 mg ADC or about 60 mg ADC, or more. In some embodiments, individual vials containing lyophilized drug product can contain ADC (e.g., anti-CD70-AS269 ADC) in an amount of about 60 mg per vial. After reconstitution of lyophilized drug product with a suitable diluent, such as water (e.g., WFI), the reconstituted formulation can have an ADC concentration of about 5 mg/mL, about 10 mg/mL, about 15 mg/mL or more. The pH can be adjusted to provide acceptable stability and administration by the skilled medical practitioner.

In some embodiments, the lyophilized drug product, when reconstituted with a diluent, provides a reconstituted solution comprising the ADC at a concentration within a range of about 5 mg/mL to about 25 mg/mL. In some embodiments, the reconstituted solution further comprises L-histidine buffer at a concentration within a range of about 10 mM to about 50 mM; sucrose at a concentration within a range of about 1% (w/v) to about 20% (w/v); and polysorbate 80 at a concentration within a range of about 0.01% (w/v) to about 0.1% (w/v). In some embodiments, the reconstituted solution has a pH within a range of about 5.4 to about 6.4. In some embodiments, the reconstituted solution has a pH within a range of about 5.2 to about 6.2. In some embodiments, the reconstituted solution pH is no greater than about 6. In some embodiments, the reconstituted solution pH is less than 6. In some embodiments, the diluent is water.

In some further embodiments, the lyophilized drug product, when reconstituted with a diluent, provides a reconstituted solution comprising the ADC (e.g., anti-CD70-AS269 ADC) at a concentration within a range of about 5 mg/mL to about 15 mg/mL, L-histidine buffer at a concentration within a range of about 10 mM to about 25 mM; sucrose at a concentration within a range of about 5% (w/v) to about 8% (w/v); and polysorbate 80 at a concentration within a range of about 0.01% (w/v) to about 0.05% (w/v). In some embodiments, the reconstituted solution has a pH within a range of about 5.5 to about 6.2. In some embodiments, the reconstituted solution pH is no greater than about 6. In some embodiments, the reconstituted solution pH is less than 6. In some embodiments, the reconstituted solution pH is about 5.5. In some embodiments, the reconstituted solution pH is about 5.6. In some embodiments, the reconstituted solution pH is about 5.7. In some embodiments, the reconstituted solution pH is about 5.8. In some embodiments, the reconstituted solution pH is about 5.9. In some embodiments, the formulation comprises the ADC at a concentration within a range of about 5 mg/mL to about 15 mg/mL, L-histidine buffer at a concentration within a range of about 10 mM to about 15 mM; sucrose at a concentration within a range of about 5% (w/v) to about 8% (w/v); and polysorbate 80 at a concentration within a range of about 0.01% (w/v) to about 0.05% (w/v), wherein the formulation has a pH within a range about 5.5 to about 6.0. In some embodiments, the reconstituted solution pH is no greater than about 6. In some embodiments, the reconstituted solution pH is less than 6. In some embodiments, the formulation contains the ADC at a concentration of about 10 mg/mL; histidine buffer at a concentration of about 20 mM; sucrose at a concentration of about 8% (w/v); and polysorbate 80 at a concentration of about 0.02% (w/v); wherein the formulation has a pH within a range of about 5.5 to about 5.9. In some embodiments, the formulation pH is no greater than about 5.9. In some embodiments, the formulation pH is less than 5.9. In some embodiments, the formulation pH is about 5.6. In some embodiments, the formulation pH is about 5.7. In some embodiments, the formulation pH is about 5.8.

The composition can be stored in a vial or cartridge, a pen delivery device, a syringe, intravenous administration tubing or an intravenous administration bag but is not limited to such. In other embodiments a pharmaceutical composition of the invention can be administered as a single dose or followed by one or more subsequent dose(s) minutes, days, or weeks after the first dose. Further administrations may be contemplated as needed to treat, reduce or prevent a cancer, condition, disorder or disease.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1: General Experimental Procedures

All commercially available anhydrous solvents are used without further purification and stored under a nitrogen atmosphere. Chromatographic purification is performed on CombiFlash Rf from Teledyne ISCO using conditions detailed in the experimental procedure.

Preparative HPLC is performed on Shimadzu system using Gemini-NX C18, 5 μm 100×30 mm, 150×30 mm or 250×50 mm column, depending on the scale. Mass spectra (MS) are recorded on a Shimadzu LCMS-2020 system and data are processed using Shimadzu LabSolutions software. Agilent 1260 Infinity Binary LC coupled with 6230 Accurate-Mass TOFMS system is used for HR-ESI-TOF analysis. NMR spectral data are collected on a 500 MHz Bruker NMR spectrometer. Chemical shifts (S) are reported in ppm and referenced off the deuterium solvent signal. Coupling constants (J) are reported in hertz (Hz). Spin multiplicities are described as: s (singlet), br (broad), d (doublet), dd (doublet of doublets), t (triplet), q (quartet) or m (multiplet).

Abbreviations used in the Examples herein: AHZ: acetohydrazide, DIAD: Diisopropyl azodicarboxylate, DMF: Dimethylformamide, HATU: 1-[Bis(dimethylamino)methylene]-1H-1,2,3-triazolo[4,5-b]pyridinium 3-oxide hexafluorophosphate, MeOH: Methanol, TFA: Trifluoroacetic acid.

Chemical names of compounds were derived from chemical structures using ChemDraw version 20.1.1 (CambridgeSoft).

### Example 2: Synthesis of Compound 1-4

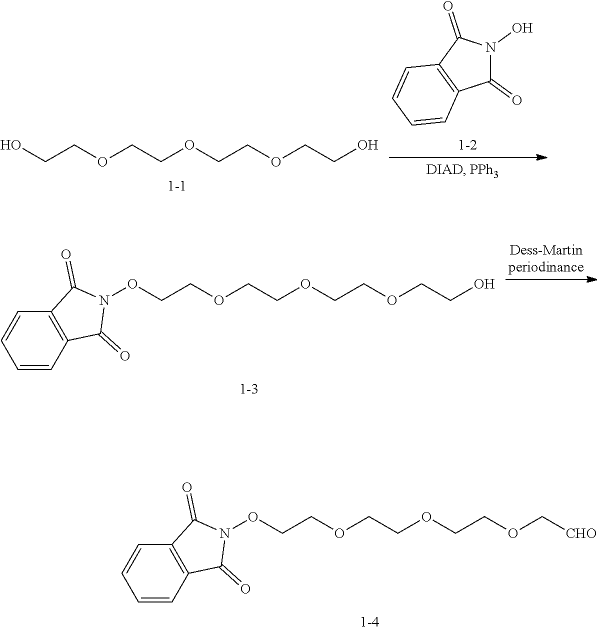 [SMILES: O=C1c2ccccc2C(=O)N1O ; OCCOCCOCCOCCO ; O=C1c2ccccc2C(=O)N1OCCOCCOCCOCCO ; O=CCOCCOCCOCCON1C(=O)c2ccccc2C1=O]

2-(2-(2-(2-(2-hydroxyethoxy)ethoxy)ethoxy)ethoxy)isoindoline-1,3-dione (Compound 1-3): Tetraethylene glycol 1-1 (10 g, 51.5 mmol), N-hydroxyphthalimide 1-2 (8.4 g, 51.15 mmol) and triphenylphosphine (17.6 g, 67 mmol) were dissolved in 300 mL of tetrahydrofuran followed by addition of DIAD (12.8 mL, 61.78 mmol) at 0° C. The resulting solution was stirred at room temperature overnight, and then concentrated to dryness. The residue was purified by flash column chromatography to give 5.47 g (31%) of compound 1-3.

2-(2-(2-(2-((1,3-dioxoisoindolin-2-yl)oxy)ethoxy)ethoxy)ethoxy)acetaldehyde (Compound 1-4): To a solution of compound 1-3 (200 mg, 0.59 mmol) in 15 mL dichloromethane was added Dess-Martin periodinane (300 mg, 0.71 mmol). The reaction mixture was stirred at ambient temperature overnight. The reaction was quenched with the solution of sodium bisulfite in 15 mL of saturated sodium bicarbonate. The mixture was separated. The organic layer was washed with saturated sodium bicarbonate, brine, dried over sodium sulfate, filtered and concentrated in vacuo. The residue was purified by flash column chromatography to give 150 mg (75%) of compound 1-4.

### Example 3: Synthesis of Compound 6

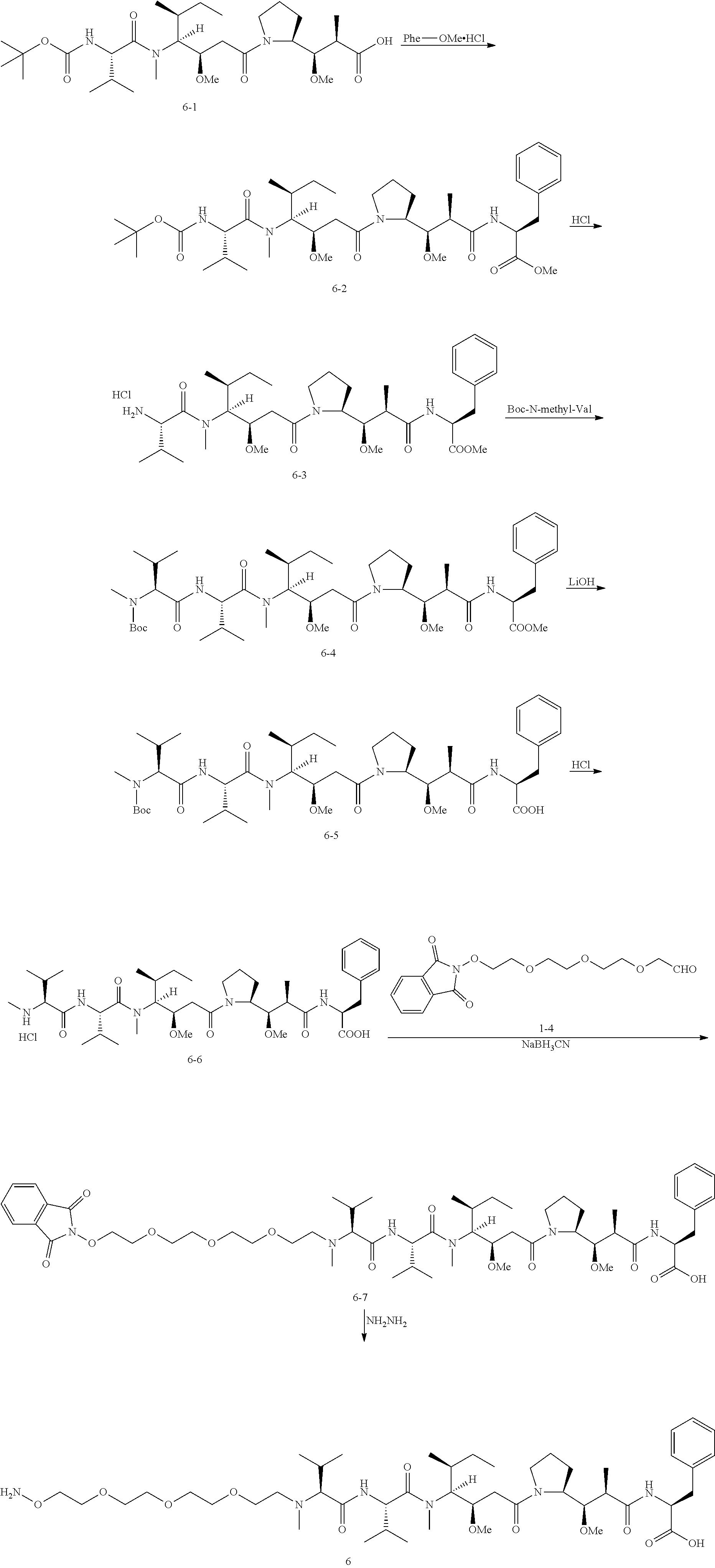

(2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanoic acid (Compound 6-1; Boc-Val-Dil-Dap-OH): Compound 6-1 is available from commercial suppliers including MedChemExpress, Monmouth Junction, NJ, Catalog No.: HY-130961.

methyl ((2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanoyl)-L-phenylalaninate (Compound 6-2): To a solution of compound 6-1 (500 mg, 0.875 mmol) in 3 mL of DMF was added 283 mg of L-phenylalanine methyl ester hydrochloride (commercially available from Sigma-Aldrich; catalog no. P17202), 433 mg of HATU and 581 μL of N-methylmorpholine. The reaction mixture was stirred at room temperature for 4 hours. The reaction mixture was concentrated in vacuo and extracted with ethyl acetate (100 mL×1, 50 mL×2). The organic layer was combined and washed with brine, dried over sodium sulfate and concentrated in vacuo. The residue was purified by flash chromatography to give 560 mg (76%) of compound 6-2.

Methyl ((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanoyl)-L-phenylalaninate (Compound 6-3): Compound 6-2 was dissolved in 15 mL 4N HCl/dioxane. The reaction mixture was stirred at room temperature for 2 hours and concentrated in vacuo to give 511 mg of compound 6-3.

methyl ((2R,3R)-3-((S)-1-((3R,4S,5S)-4-((S)-2-((S)-2-((tert-butoxycarbonyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalaninate (Compound 6-4): To a solution of compound 6-3 (368 mg, 0.55 mmol) in 3 mL of DMF was added 255 mg of Boc-N-methyl-L-valine (commercially available from Sigma-Aldrich; catalog no. 15538), 314 mg of HATU and 303 μL of N-methylmorpholine. The reaction mixture was stirred at room temperature for 4 hours. The reaction mixture was concentrated in vacuo and extracted with ethyl acetate (100 mL×1, 50 mL×2). The organic layer was combined and washed with brine, dried over sodium sulfate and concentrated in vacuo. The residue was purified by flash chromatography to give 370 mg (79%) of compound 6-4.

((2R,3R)-3-((S)-1-((3R,4S,5S)-4-((S)-2-((S)-2-((tert-butoxycarbonyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine (Compound 6-5): To a solution of compound 6-4 (170 mg) in 10 mL MeOH was added 5 eq of 1N LiOH. The reaction mixture was stirred at room temperature for 2 hours. The reaction mixture was acidified by 1N HCl and extracted with ethyl acetate washed with brine, dried over sodium sulfate and concentrated in vacuo to give 150 mg (90%) of compound 6-5.

((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanoyl)-L-phenylalanine (Compound 6-6): Compound 6-5 was dissolved in 4N HCl/dioxane. The reaction mixture was stirred at room temperature for 2 hours and concentrated in vacuo and purified by HPLC to give 150 mg of compound 6-6.

((2R,3R)-3-((S)-1-((3R,4S,5S)-4-((S)-2-((S)-14-((1,3-dioxoisoindolin-2-yl)oxy)-2-isopropyl-3-methyl-6,9,12-trioxa-3-azatetradecanamido)-N,3-dimethylbutanamido)-3-methoxy-5-methylheptanoyl)pyrrolidin-2-yl)-3-methoxy-2-methylpropanoyl)-L-phenylalanine (Compound 6-7): To a solution of compound 6-6 (50 mg, 0.062 mmol) in 1 mL of DMF was added compound 1-4 (63 mg, 0.186 mmol) and 70 μL of acetic acid, followed by addition of 8 mg of sodium cyanoborohydride. The resulting mixture was stirred at ambient temperature for 2 hours. The reaction mixture was diluted with water and purified by HPLC to give 60 mg (80%) of compound 6-7.

((2R,3R)-3-((S)-1-((3R,4S,5S)-4-((S)-2-((S)-14-(aminooxy)-2-isopropyl-3-methyl-6,9,12-trioxa-3-azatetradecanamido)-N,3-dimethylbutanamido)-3-methoxy-5-methylheptanoyl)pyrrolidin-2-yl)-3-methoxy-2-methylpropanoyl)-L-phenylalanine (Compound 6): Compound 6-7 (60 mg, 0.05 mmol) was dissolved in 1 mL of DMF. Hydrazine (32 μL) was added. The resulting solution was stirred at ambient temperature for 1 hour. The reaction was quenched with 1N hydrochloride solution. The reaction mixture was purified by HPLC to give 33 mg (55%) of compound 6.

### Example 4

Methods of generating cell lines to promote non-natural amino acid-containing protein production using genome engineering technology, essentially as described in WO2018/223108, the entire contents of which are hereby incorporated by reference in their entirety, can be applied to generate anti-CD70 antibodies containing non-naturally encoded amino acids of the present disclosure.

Molecular Cloning for expression of anti-CD70 antibodies. CHO cell codon-optimized antibody heavy chain and light chain cDNA sequences were obtained from a commercial DNA synthesis service (Integrated DNA Technologies (IDT), San Diego, CA). The synthesized DNA fragments were digested with Hind III and EcoR I (both from New England BioLabs, (NEB), Ipswich, MA) and purified using a PCR purification kit (Qiagen, Valencia, CA). Then the digested antibody gene fragments were ligated into the expression vector via a quick ligation kit (NEB) to yield the constructs for expression of wild type antibody heavy chain and light chain. The resulting plasmids were propagated in E. coli and verified by a DNA sequencing service (Eton Biosciences, San Diego, CA).

Generation of amber codon-containing mutants. Based on the crystal structure of an immunoglobulin G1 (IgG1) mAb, heavy chain amino acid A114 (Kabat numbering) was chosen to genetically incorporate non-natural amino acid para-acetyl-L-phenylalanine (pAF). Alternative Fc mutations were chosen for optimization of stability pharmacokinetics, antibody-dependent cellular phagocytosis (ADCP) and/or antibody-dependent cellular cytotoxicity, including heavy chain amino acid A136 and L159. Light chain amino acid mutation sites were selected as alternatives to heavy chain mutations and/or to serve as additional conjugation sites to provide ADCs with higher drug-to-antibody ratios. The genetic codon of the chosen site was then mutated to amber codon (TAG) via site-directed mutagenesis to generate an expression plasmid for that antibody mutant. Site-directed mutagenesis experiments were carried out using Q5 site-directed mutagenesis kits from New England Biolabs (NEB). The expression plasmids for the mutants were propagated in E. coli and verified by a DNA sequencing service (Eton Biosciences).

Table 1 provides a list of amino acid sequences, including amber mutations sites (Kabat numbering) in the heavy chain or light chain of anti-CD70 antibodies. SEQ ID NOs: 1 and 2 show wild type heavy chain and light chain amino acid sequences, respectively. Anti-CD70 mAb light chains with non-naturally encoded amino acids include the light chain sequences of: SEQ ID NO: 6; SEQ ID NO: 7; SEQ ID NO: 8; and SEQ ID NO: 9. Anti-CD70 mAb heavy chains with non-naturally encoded amino acids include the heavy chain sequences of: SEQ ID NO: 3, SEQ ID NO: 4; and SEQ ID NO: 5.

### Protocols for Production of Anti-CD70 Antibodies Containing pAF at Heavy Chain Position 114 (Kabat Numbering)

Transient expression. Platform cell line was maintained in EX-Cell 302 (Sigma) supplemented with 3 mM L-glutamine (Gibco) and 3 mM GlutaMAX (Gibco). Cells were passaged every 3 to 4 days seeded at a density of 0.4 million cells per ml. One day prior to transfection, cells were seeded at 0.6 million cells per ml. On day 0, cells were transfected with antibody expression plasmids encoding the light chain and heavy chain using MaxCyte electroporation platform following the instruction manual. After transfection, cells were rested in an empty 125 ml shake flask and incubated at 37° C. in a static incubator for 30 mins. The transfected cells were then inoculated into basal expression media (50% Dynamis:50% ExCell 302 supplemented with 3 mM L-glutamine and 3 mM GlutaMAX) at a density of 3×10⁶/ml in shake flask. The transfected cells were incubated at 37° C., 5% CO₂ on an orbital shaker set to 140 rpm. The following were added to the culture on day 1: pAF (final concentration in culture: 1 mM), Cell Boost 5 (GE Healthcare; final concentration in culture: 7 g/L), Long R3 IGF-1 (Sigma; final concentration in culture: 120 μg/L) and GlutaMAX (final concentration in culture: 2 mM). The temperature was shifted from 37° C. to 32° C. inside the incubator. Additional Cell Boost 5 (final concentration: 7 g/L) and GlutaMAX (final concentration: 2 mM) was added on day 3 and supernatant was collected on day 5. The culture media glucose level was monitored using glucose meters, and additional glucose was added to the culture when the glucose level was below 2 g/L. Viable cell count and viability were measured by Vi-Cell instrument. Antibody production was measured by Octet using Protein G sensors.

Stable bulk pool generation. The expression plasmid was linearized using Pvu I (NEB) digestion for four hours. After linearization, the DNA was purified using phenol:chloroform:isoamyl alcohol extraction and dissolved in endotoxin-free water at the concentration of 2.5 μg/μl. Platform cell line was maintained in EX-Cell 302 supplemented with 3 mM L-glutamine and 3 mM GlutaMAX. Cells were passaged every 3 to 4 days seeded at a density of 0.3×10⁶/ml. One day prior to transfection, cells were seeded at 0.6×10⁶/ml. On day 0, 15×10⁶ cells were transfected with 25 μg of linearized antibody expression plasmids using MaxCyte electroporation (OC-100) platform following the instruction manual. After transfection, cells were rested in an empty 125 ml shake flask and incubated at 37° C. in a static incubator for 30 mins. Then 30 ml recovery media (50% Ex-302:50% CD-CHO supplemented with 3 mM L-glutamine and 3 mM GlutaMAX) was added into the flask and shaked overnight. On day one, transfected cells were counted, spun down, washed and re-suspended in selection media (50% EXCELL 302:50% CD-CHO with 50 μM MSX) for stable bulk pool generation. The viable cell numbers and viability were monitored, and media was changed every 3 to 4 days until the viability of the stable bulk pool returned to 90%. When selection ended, frozen cell stocks were made, and the resulting stable bulk pool was used to generate material for fed-batch expression.

Fed-batch expression. Previously generated antibody stable bulk pools were inoculated into basal expression media (50% Dynamis:50% ExCell 302 supplemented with 50 μM MSX) at a density of 0.5×10⁶/ml in a shake flask on day 0. The transfected cells were incubated at 37° C., 5% CO₂ on an orbital shaker set to 150 rpm. The following were added to the culture on day 3: pAF (CAS #122555-04-8; final concentration in culture: 0.5 mM), Cell Boost 4 (GE Healthcare; final concentration in culture: 10 g/L) and Cell Boost 7b (GE Healthcare; final concentration in culture: 0.52 g/L). Long R3 IGF-1 (final concentration in culture: 120 μg/L) was added to the culture on day 5. The culture media glucose level was monitored using glucose meters, and additional glucose was added to the culture when the glucose level was below 2 g/L. Viable cell count and viability were measured by Vi-Cell instrument. The supernatant was collected for purification on day 7. Antibody production was measured by Octet using Protein G sensors.

Purification of Antibodies Containing nnAAs from EuCODE Expression System.

Clarified Cell culture media containing the target antibody containing non-naturally encoded amino acid was loaded over a protein A ProSep Ultra column (EMD Millipore) equilibrated in 20 mM sodium phosphate, 100 mM sodium chloride, pH 7.5. After loading, the column was washed with buffer A (20 mM sodium phosphate, 100 mM sodium chloride, pH 7.5) followed by wash buffer B (5 mM succinic acid, pH 5.8) to remove host cell contaminants. The target antibody was eluted from the column with elution buffer C (50 mM glycine, 10 mM succinic acid, pH 3.2). The target antibody was pooled, and pH adjusted to pH 5.0 with 2.0 M tris base. The target antibody was further purified by loading the conditioned protein A pool over a Capto SP Impres column (GE Healthcare) equilibrated in 30 mM sodium acetate, pH 5.0. The target antibody was eluted from the column with a linear gradient to 100% buffer B (30 mM sodium acetate, 0.5 M sodium chloride, pH 5.0) and fractions containing monomeric antibody were pooled, 0.22 μM filtered, and stored at ≤65° C. until further use.

### Example 5: Anti-CD70-AS269 ADC Synthesis

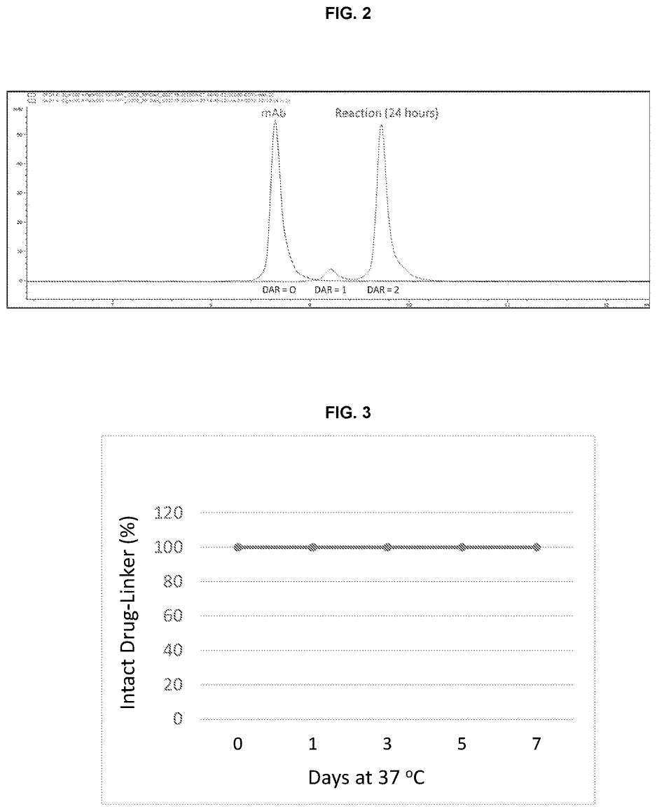

AS269 (FIG. 1) was conjugated to a humanized anti-CD70 monoclonal antibody (mAb), subclass immunoglobulin G1 (IgG1) with specificity for human CD70 and containing non-naturally encoded amino acid para-acetyl-L-phenylalanine at position 114 (Kabat numbering) of each heavy chain sequence, in the presence of acetohydrazide (AHZ) under controlled reaction conditions. Briefly, stock solutions of AS269 and AHZ were prepared with water for injection (WFI) and added to anti-CD70 mAb solution at molar ratios of 8:1 (AS269:mAb) and 300:1 (AHZ:mAb). After addition, the pH was adjusted to 3.8-4.3 using 0.5 M citrate buffer and diluted to 30±2.0 g/L. The conjugation reaction proceeded for 24-28 hours at 22±2° C. with continuous, gentle mixing. Reversed phase (RP) chromatography was used to monitor the reaction progress and ensure that a drug-to-antibody ratio (DAR) of 1.8-2.0 was met before further processing. RP-HPLC analysis was performed on an Agilent 1200 series HPLC system using an Agilent Stablebond SB—C8, 5 μm, 4.6×150 mm column. Mobile phase A consisted of 0.1% TFA in water and mobile phase B consisted of 0.1% TFA in acetonitrile. The flow rate was 1 mL/minute, the column temperature was 75° C., and detection was recorded at A280 nm. mAb and ADC elution occurred during a 30-60% gradient increase of mobile phase B over 20 minutes. A representative HPLC chromatogram (FIG. 2) shows unconjugated mAb prior to conjugation, and anti-CD70-AS269 ADC (DAR=1 and DAR=2) after 24 hours.

After conjugation, the reaction mixture containing the anti-CD70-AS269 ADC was diafiltered (ten (10) diafiltration volumes) into a histidine formulation buffer via tangential flow filtration (TFF) using a 30 kDa molecular weight cut-off membrane. Solutions of histidine buffer, pH 5.7, sucrose, and polysorbate 80 (PS80) were used to formulate the diafiltered ADC to provide a final formulation containing 10.0 mg/mL ADC (active substance), histidine buffer, sucrose and PS80, pH 5.7. The formulated ADC was 0.22-micron filtered, filled into polycarbonate (PC) bottles and stored frozen at −70±10° C. The frozen solutions were thawed, aliquoted into glass vials, and subsequently freeze-dried to a white lyophilized cake. The lyophile is reconstituted with WFI prior to administration.

The following scheme shows an exemplary ADC engineered by conjugating AS269 (Compound 6) with anti-CD70 mAb comprising non-naturally encoded amino acid pAF:

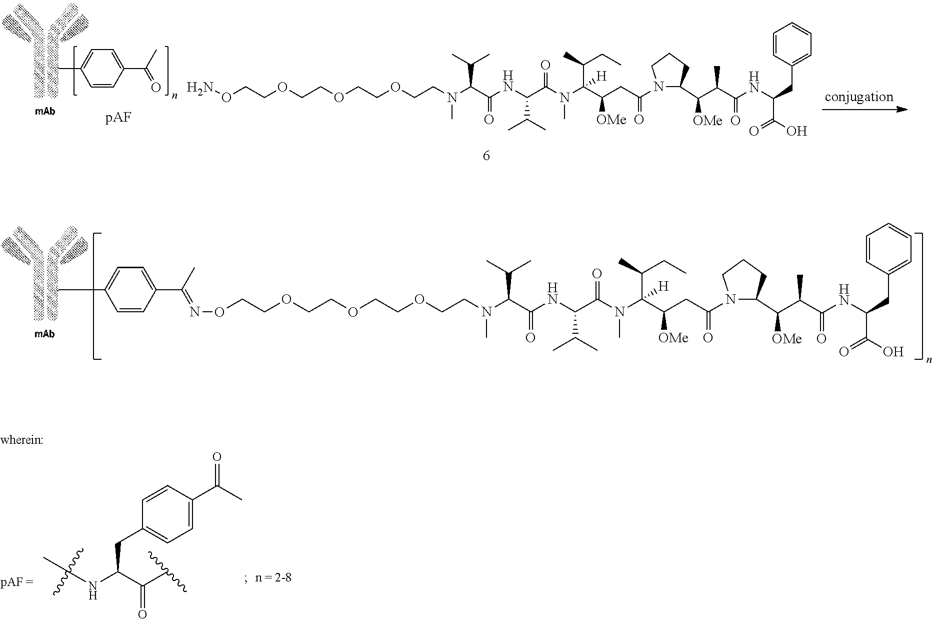

### Example 6: Anti-CD70-AS269 ADC Ex Vivo Mouse Plasma Stability

Anti-CD70-AS269 ADC was incubated in mouse plasma for 7 days at 37° C. After T=0, 1, 3, 5 and 7 days incubation, samples were processed to capture antibody, extracted, and analyzed by LC-MS for intact AS269 drug-linker on the heavy chain of the antibody. The percent intact drug-linker was determined relative to the T=0 timepoint. Anti-CD70-AS269 ADC exhibited stability with 100% intact drug-linker after 7 days incubation in mouse plasma (FIG. 3).

### Example 7: Anti-CD70-AS269 ADC Pharmacokinetics in CD-1 Mice

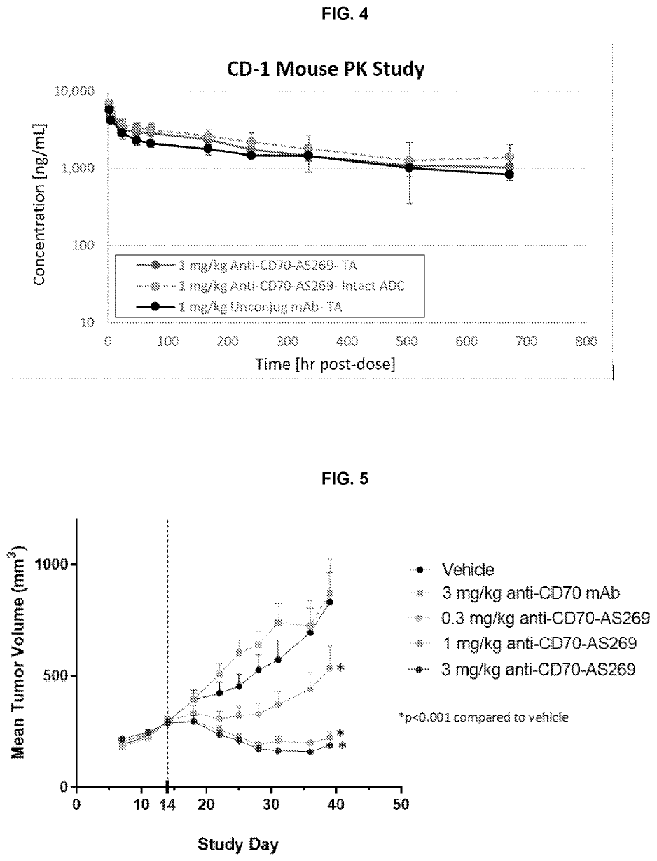

A single dose of 1 mg/kg anti-CD70-AS269 ADC or 1 mg/kg of Unconjugated mAb was administered to CD-1 mice (n=5/group) and a small volume of blood was collected from all animals at pre-dose and 2, 6, 24, 48, 96, 168, 240, 336, 504 and 672 hours post-dose. Blood samples were immediately diluted into Casein Blocker buffer in tubes and frozen at ≤60° C. Samples were analyzed in a Total Antibody (TA) pharmacokinetics method that detects the antibody scaffold (unconjugated and conjugated species), and the anti-CD70-AS269 ADC samples were also measured in an intact ADC pharmacokinetics method that only detects ADC species having a drug-antibody ratio of 2 (DAR=2). Any loss of drug-linker from the anti-CD70-AS269 ADC would result in separation of the ADC TA and Intact ADC concentration-time curves. The ADC TA and Intact ADC curves were in good agreement, demonstrating anti-CD70-AS269 is stable in mouse circulation through 672 hours and has a long ADC terminal half-life of 397 hours (FIG. 4). Additionally, the anti-CD70-AS269 ADC TA and Intact ADC curves were similar to the Unconjugated mAb TA curve, suggesting the anti-CD70-AS269 ADC clearance is similar to unconjugated mAb and not impacted by conjugation of the AS269 drug-linkers.

### Example 8: 786-O S3 Renal Carcinoma Xenograft Model in Mice

Mice. Nu/nu female mice, 6-7 weeks of age were received from Charles River Laboratories and housed 5 per cage in a barrier, pathogen-free, limited access room. Animals had access to gamma irradiated diet (Newco Item #15061) and autoclaved tap-water ad libitum. Mice were identified individually by tail tattoo and were given a minimum of 3 days to acclimate prior to initiation of study activities.

Cells. 786-O renal carcinoma cells were purchased from ATCC (cat #CRL-1932) and serially passaged in nude mice 3 times for faster growth. 786-O S3 Cells were cultured in vitro in RPMI+10% FBS+P/S for a minimum of 2 weeks prior to implant. Freshly harvested 786-O S3 cells were suspended in PBS and mixed 1:1 with matrigel. Mice were anesthetized with isoflurane anesthesia (2-3%, 2 L/min oxygen) and implanted subcutaneously in the right flank with 5e6 cells/mouse in 0.2 mL cell suspension.

In-life Measurements and Study Design. On day 14, when tumors were an average size of 300 mm3, mice were sorted into groups of 10 mice each. The vehicle group received formulation buffer, and treatment for all groups was administered IV at 10 mL/kg. Tumor sizes were measured by calipers consisting of length (across longest line of tumor) and width (perpendicular to length measurement). Tumor volume was calculated as: L×W×W×0.5. Data shown are average tumor volumes ±SEM for each cohort over time (FIG. 5).

### Example 9: 786-O S3 Renal Carcinoma Xenograft Model in Mice—Anti-CD70-AS269 ADC Vs Sunitinib

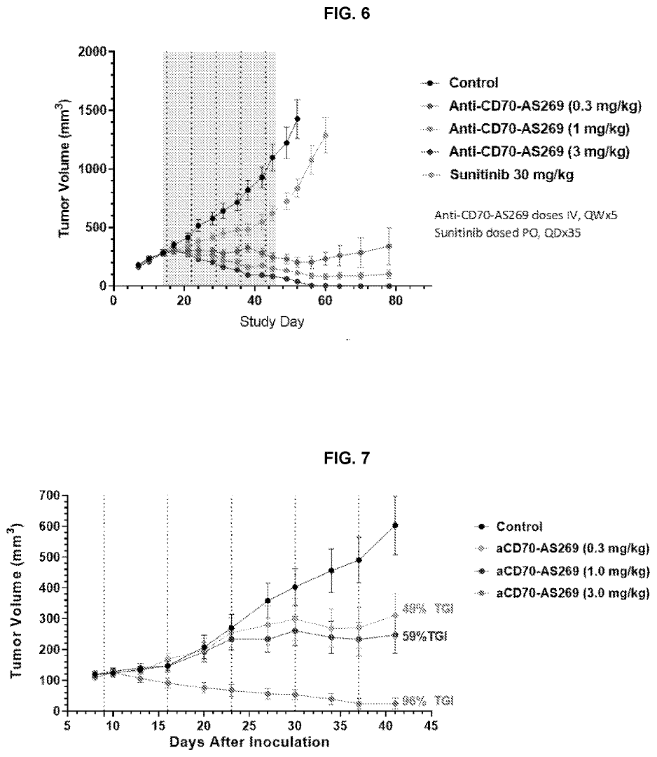

786-O S3 cells were implanted subcutaneously into female nu/nu mice and tumor growth monitored as described in Example 8. Three different dose levels of anti-CD70-AS269 ADC were administered IV to mice (n=10/group) once per week (dotted lines) for 5 total doses, and 30 mg/kg sunitinib was administered PO daily for 35 days. Data shown are average tumor volumes ±SEM for each cohort over time (FIG. 6).

### Example 10: Caki-1 Renal Carcinoma Xenograft Model in Mice

Mice. Taconic Laboratories NCRNU—F CrTac:NCR-Foxn1<nu> female mice 4 weeks of age were housed 5 per cage in a barrier, pathogen-free, limited access room. Food was gamma-irradiated diet (Newco Item #15061) and autoclaved tap-water was provided ad libitum. Mice were identified individually by ear tags.

Cells. Caki-1 renal cell carcinoma cells were purchased from ATCC and cultured in McCoys+10% FBS+P/S for a minimum of 2 weeks prior to implant. Cells were implanted at passage number 5 with 5e6 cells inoculated per mouse in the right rear flank.

In-life Measurements and Study Design. On day 9 post-implantation, when tumors were an approximate average size of 115 mm³, mice were sorted into 4 groups of 10 mice each. The test articles indicated in FIG. 7 were administered once weekly (dotted lines) and tumor volumes measured as described in Example 8. Data shown are average tumor volumes ±SEM for each cohort over time (FIG. 7).

### Example 11: Caki-1 Renal Carcinoma Xenograft Model in Mice—Individual Tumor Volumes

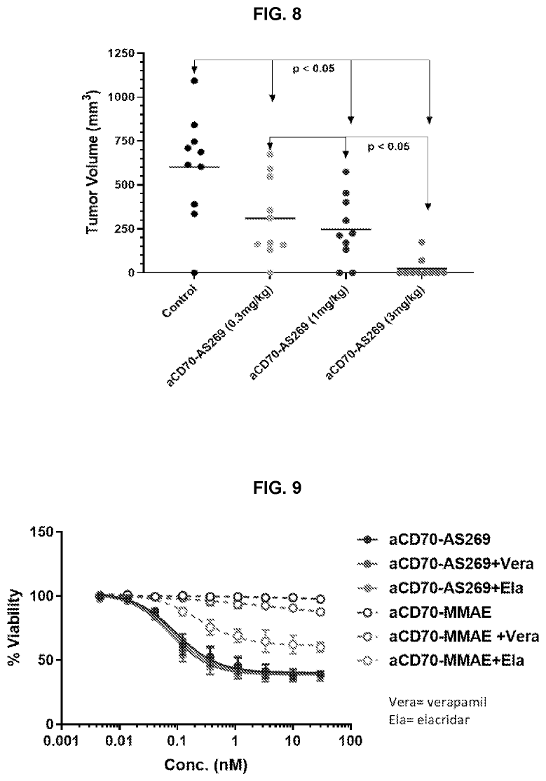

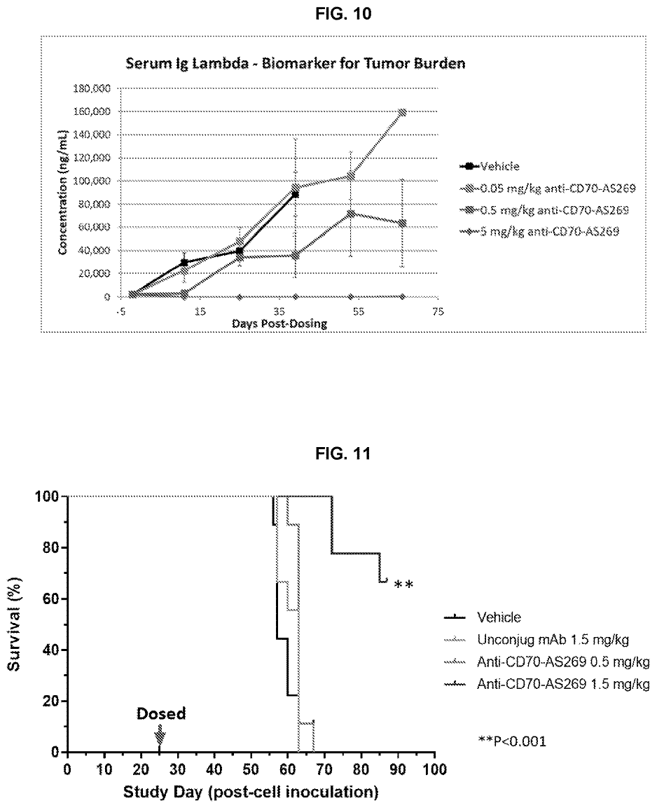

Individual Caki-1 tumor volumes on day 41 with the mean value of each treatment group represented by the solid line are shown in FIG. 8 from the repeat-dose Caki-1 xenograft study described in Example 10.

### Example 12: Viability of MDR-Positive 786-O Cells Treated with Anti-CD70 ADCs

786-O cells were seeded at 1,000 cells/80 μL/well in a 96-well white plate and incubated overnight in a 37° C., 5% CO₂ incubator. The next day, 5× concentrated anti-CD70-AS269 or anti-CD70-MMAE was prepared in a regular media or 5× concentrated elacridar- or verapamil-containing media. The final concentrations of elacridar and verapamil in the assays was 1 μM and 4 μM, respectively. Serially diluted ADCs (20 μL) were added to the wells, and the plates were incubated in a 37° C., 5% CO₂ incubator for 96 hours. At the end of incubation, CellTiter-Glo2.0 (Promega, Madison, WI) was added to the room-temperature equilibrated plates and luminescence was measured on a SpectraMax M5E luminometer. Cell viability was calculated as a percentage of ADC-untreated control. The IC₅₀ was determined by nonlinear 4-parameter dose-response curve fitting using GraphPad Prism (GraphPad Software, San Diego, CA). Anti-CD70-AS269 ADC exhibited strong activity against MDR-positive 786-O cells, whereas control anti-CD70-MMAE ADC exhibited poor activity that improved with the addition of MDR-inhibitor elacridar (FIG. 9). [Monomethyl auristatin E (MMAE) is a known substrate for MDR pumps. AS269 was not a substrate for MDR/P-gp or BCRP (data not shown).]

### Example 13: Anti-CD70-AS269 Efficacy in U266 Multiple Myeloma Disseminated Model

Male NOG mice (n=9/group) were injected intravenously with U266 cells, and IgE lambda serum levels were measured as an indicator of tumor burden. Once IgE lambda levels reached approximately 4500 ng/mL, animals were sorted into groups and administered 0.5 mg/mg or 1.5 mg/kg anti-CD70-AS269 or 1.5 mg/kg unconjugated mAb. Animals were weighed twice a week and monitored daily for survival. Mice were also bled once every two weeks by tail snip with 50-100 μL blood collected into serum separator tubes and then measured in an IgE lambda ELISA. A single dose of 1.5 mg/kg Anti-CD70-AS269 significantly extended survival (FIG. 11) and also delayed tumor burden and reduced body weight loss compared to vehicle (data not shown).

### Example 14: Co-Culture CD27 Reporter Assay

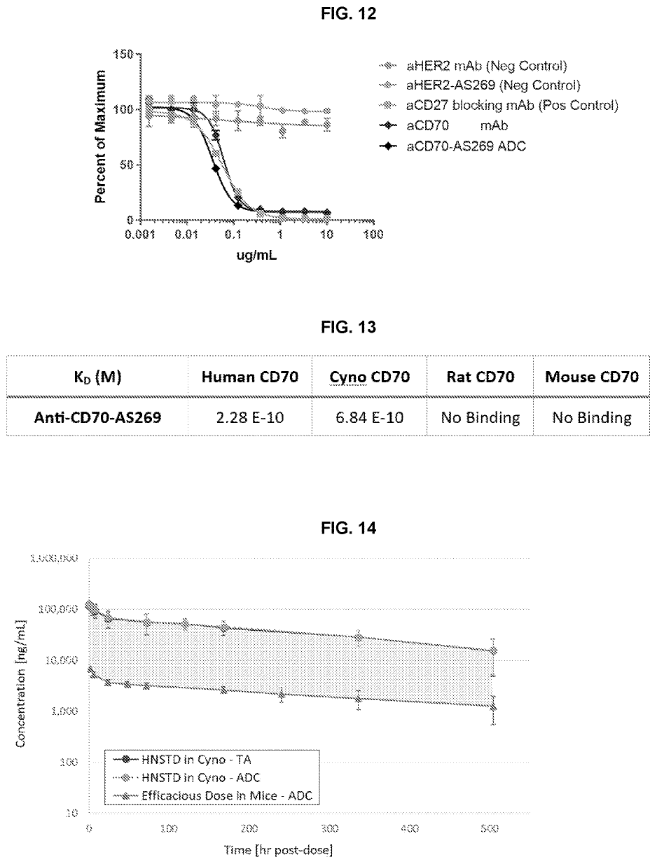

786-O cells were seeded at 40,000 cells/well in a 96-well plate and incubated overnight in an incubator set to 37° C., 5% CO₂. The next day, a vial of Jurkat/CD27/NFkB-luciferase (Jurkat NFkB-luc) reporter cells was thawed, and cells were resuspended in Assay Buffer. The Jurkat NFkB-luc reporter cells were added to the seeded 786-O cells along with serially diluted anti-CD70-AS269 ADC or the parental anti-CD70 mAb, negative control anti-HER2-AS269 ADC or anti-HER2 mAb, or a positive control blocking anti-CD27 mAb. The 96-well co-culture plate was incubated at 37° C., 5% CO₂, and the luminescence signal was detected in a luminometer. The anti-CD70 mAb, anti-CD70-AS269 ADC and positive control blocking anti-CD27 mAb all inhibited CD70-mediated CD27 signaling in the Jurkat NFkB-luc reporter cells, but the negative control anti-HER2 mAb and anti-HER2-AS269 ADC did not inhibit signaling (FIG. 12).

### Example 15: In Vitro Activity and CD70 Expression Across Multiple Cell Types

In Vitro Activity. All cell lines, except for U266 and NCI-H929, were seeded into 96-well cell culture plates at optimized cell densities (ranging from 1,000-8,000 cells/well) for 5-day incubation and incubated overnight in an incubator set to 37° C., 5% CO₂. U266 and NCI-H929 cells were seeded at optimized cell densities for 4-day incubation and were not incubated overnight in an incubator. Anti-CD70-AS269 ADC was serially diluted in the appropriate cell line-specific culture medium and directly added in duplicate or triplicate to the seeded cells. The total volume in the assay wells was 100 μL/well. The assay plates were incubated in an incubator set to 37° C., 5% CO₂. After four days incubation, plates were equilibrated to room temperature and cell viability was measured by addition of Promega CellTiter-Glo or CellTiter-Glo2.0 Reagent, incubation for 2 minutes on a plate shaker to induce cell lysis, incubation for 10 minutes at room temperature to stabilize signals, and readout on a luminometer. Cell viability was calculated as a percentage of untreated control cells, and the anti-CD70-AS269 IC₅₀ values were determined using a 4-parameter curve fit of the dose response curves.

CD70 Expression Levels. CD70 cell surface number in the various cell lines was quantified using QiFiKit (Dako, cat #K0078). Cell lines were harvested and stained with mouse anti-human CD70 Ki-24 antibody (BD Biosciences cat #555833) for 1 hour at 4° C. After washing, cells and QiFiKit control beads were stained with anti-mouse IgG-FITC secondary antibody (1:50 dilution) for 45 minutes at 4° C., washed twice, and analyzed on a flow cytometer in the FITC channel. CD70 number per cell was calculated using the reference curve generated with the QiFiKit control beads.

Anti-CD70-AS269 generally showed higher activity in tumor cell lines expressing higher CD70/cell and lower activity in tumor cell lines expressing lower CD70/cell, demonstrating the specificity of the anti-CD70-AS269 ADC (Table 2).

TABLE 2
Anti-CD70-AS269 specificity and potency across multiple cell lines.

| | | CD70 | Cytotoxic | Maximum |
|---|---|---|---|---|
| | | Surface | Potency | Cytotoxicity |
| Cell Lines | Tumor Type | Expression | (IC₅₀, nM) | (Emax, %) |
| 786-O | Renal cancer | 133,000 | 0.167 | 48.0 |
| Caki-1 | Renal cancer | 107,000 | 0.125 | 52.0 |
| U251MG | Brain cancer | 65,800 | 0.040 | 60.8 |
| U266 | Myeloma | 65,000 | 0.025 | 82.0 |
| Raji | Burkitt's lymphoma | 17,700 | >10 | 33.0 |
| REC-1 | Mantle Cell Lymphoma | 17,300 | 0.180 | 54.0 |
| NCI-H40 | Lung cancer | 14,900 | 0.180 | 46.6 |
| Hs766T | Pancreatic cancer | 9,500 | >10 | 10.8 |
| KMS11 | Myeloma | 9,400 | >10 | 15.4 |
| C666-1 | Nasopharyngeal cancer | 9,200 | >10 | −3.1 |
| Hs 611.T | Hodgkin's lymphoma | 6,600 | >10 | 4.3 |
| NCI-H929 | B lymphoblast | 0 | >10 | 15.7 |

### Example 16: Anti-CD70-AS269 ADC Affinity for CD70 from Various Species

Surface plasmon resonance assays were developed in which anti-human IgG (Fc) was diluted to 25 μg/mL in immobilization buffer (10 nM Sodium Acetate, pH 5.0) and injected to an activated CM5 sensor chip for 360 seconds at a flow rate of 10 μL/minute. Anti-CD70-AS269 ADC was then diluted to 5 or 20 μg/mL in running buffer (1×HEPES+0.005% Tween-20, pH 7.4) and injected into the sample channel. Analytes human CD70, cynomolgus CD70, rat CD70, or mouse CD70 proteins were serially diluted in the running buffer and injected at a flow rate of 30 μL/minute for 120-second association and 300-second dissociation phases. After each cycle of interaction analysis, the sensor chip surface was regenerated with 3M magnesium chloride at a flow rate of 20 uL/minute for 30 seconds. Affinity analysis was performed using Biacore Insight Evaluation software, reference channel was used for background subtraction, and a 1:1 binding method was used for curve fitting. Anti-CD70-AS269 bound human and cynomolgus CD70 within 3-fold, but showed no cross-reactivity to rodent CD70 (FIG. 13).

### Example 17: Anti-CD70-AS269 ADC Toxicokinetics and Pharmacokinetics

Anti-CD70-AS269 ADC was administered intravenously at different dose levels to male and female cynomolgus monkeys (n=5/sex/dose level). The ADC was administered every 3 weeks for a total of 3 doses (1.5 mg/kg, 4.5 mg/kg and 9 mg/kg), followed by a 5-week recovery period. Serum samples were collected from animals at various time points and measured in Total Antibody (TA) and Anti-CD70-AS269 ADC ligand-binding assays with Meso Scale Discovery (MSD) readout. Comparison of the Anti-CD70-AS269 ADC toxicokinetic (TK) concentration-time curve at the highest nonseverely toxic dose (HNSTD) concentration in monkeys (4.5 mg/kg) versus the Anti-CD70-AS269 Intact ADC pharmacokinetic concentration-time curve at a pharmacologically active dose in mice (see Example 7) shows a clear therapeutic index (FIG. 14).

### Example 18: Phase I Dose-Escalation and Dose Expansion Study of ARX305, an

Anti-CD70-AS269 ADC, in Subjects with Relapsed or Refractory Clear Cell Renal Cell Carcinoma. A first-in-human (FIH) multicenter, open label, dose-escalation and dose-expansion study can evaluate the safety, pharmacokinetics (PK) and anti-tumor activity of ARX305 in subjects with clear cell renal cell carcinoma (ccRCC) whose tumors are resistant or refractory to prior standard therapies. Subjects must have been previously treated with kinase inhibitor, anti-angiogenic agent, mTOR inhibitor, and/or immune checkpoint inhibitor (as monotherapy or in combination).

The study includes a dose-escalation part and a dose-expansion part in patients with relapsed or refractory ccRCC. ARX305 is administered intravenously in Q3W cycle, and cycles continues as long as the subject is eligible to continue treatment. Subjects receive ARX305 at 0.24 mg/kg as the initial dose on Day 1 of the first 3-week cycle followed by escalating doses in the Phase 1 trial. Examples of escalating doses may include about 0.05 mg/kg, about 0.1 mg/kg, about 0.12 mg/kg, about 0.14 mg/kg, about 0.16 mg/kg, about 0.18 mg/kg, about 0.2 mg/kg, about 0.22 mg/kg, about 0.24 mg/kg, about 0.26 mg/kg, about 0.28 mg/kg, about 0.3 mg/kg, about 0.32 mg/kg, about 0.34 mg/kg, about 0.36 mg/kg, about 0.38 mg/kg, about 0.4 mg/kg, about 0.42 mg/kg, about 0.44 mg/kg, about 0.46 mg/kg, about 0.48 mg/kg, about 0.5 mg/kg, about 0.52 mg/kg, about 0.54 mg/kg, about 0.56 mg/kg, about 0.58 mg/kg, about 0.6 mg/kg, about 0.62 mg/kg, about 0.64 mg/kg, about 0.66 mg/kg, about 0.68 mg/kg, about 0.7 mg/kg, about 0.72 mg/kg, about 0.74 mg/kg, about 0.76 mg/kg, about 0.78 mg/kg, about 0.8 mg/kg, about 0.82 mg/kg, about 0.84 mg/kg, about 0.86 mg/kg, about 0.88 mg/kg, about 0.9 mg/kg, about 0.92 mg/kg, about 0.94 mg/kg, about 0.96 mg/kg, about 0.98 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg or about 2 mg/kg of the human subject. More particular doses may include about 0.24 mg/kg, 0.48 mg/kg, 0.8 mg/kg, 1.1 mg/kg, 1.3 mg/kg, and 1.5 mg/kg. Safety, efficacy and objective response rate (ORR) are assessed using RECIST 1.1 criteria. ARX305 is administered as monotherapy, and no other anticancer agents are administered in this study.

### Example 19: ARX305 Lyophilized Powder for Injection

ARX305 formulation development included chemical and physical characterization of the ARX305 antibody-drug conjugate drug substance (e.g., amino acid sequence, charge, isoelectric point (pI), molecular weight, drug-to-antibody ratio (DAR), size and charge variants), solubility, excipient screening (e.g., excipient identities and concentrations, buffer pH, identity and concentration) and stability studies, and consideration of the ability to minimize manufacturing processing steps between bulk drug substance and formulated drug product. The lead clinical formulation was selected based on outcomes of evaluations of the foregoing, including solubility (protein recovery), chemical and physical stability criteria using high performance liquid chromatography (HPLC) analysis and capillary electrophoresis.

The excipients were selected according to their stabilizing effect on the drug product. L-histidine and L-histidine monohydrochloride monohydrate, at a concentration of 20 mM, maintains the pH in the liquid state. Sucrose, at a concentration of 8% (w/v), stabilizes ARX305 active substance against aggregate formation in the liquid state and serves as a cryoprotectant. Polysorbate 80, at a concentration of 0.02% (w/v), was chosen to stabilize ARX305 active substance against agitation in the liquid state.

ARX305 active drug substance, at a target protein concentration of 10 mg/mL in 20 mM histidine buffer, 8% (w/v) sucrose, and 0.02% (w/v) polysorbate 80 at pH 5.7, is sterile filtered, filled into 20 mL glass vials, and freeze-dried in a lyophilizer to produce the drug product.

The clinical formulation for IV administration, ARX305 Injection, Lyophilized Powder (hereinafter, ARX305 Drug Product), contains ARX305 antibody-drug conjugate (drug substance; 60 mg/vial) formulated at a concentration of 10 mg/mL in a solution of histidine buffer (L-histidine, L-histidine hydrochloride) at a concentration of 20 mM, sucrose at a concentration of 8% (w/v) and polysorbate 80 at a concentration of 0.02% (w/v); the final solution pH prior to lyophilization (and after reconstitution of the lyophile) is pH 5.7. See Table 3. The labeled fill volume is 6.0 mL per vial. Based on extractable volume determination study, the overfill volume is set at 0.46 mL per vial.

TABLE 3
Composition of ARX305 Drug Product

| | Amount per | | Quality |
|---|---|---|---|
| Component | Vial ¹ | Function | Standard |
| ARX305 Antibody | 60 mg | Active | In-house |
| Drug Conjugate | | Ingredient | specification |
| L-Histidine | 5.52 mg | Buffer | Ph. Eur./USP |
| L-Histidine | 17.70 mg | Buffer | Ph. Eur. |
| monohydrochloride | | | |
| monohydrate | | | |
| Sucrose | 480.0 mg | Cryoprotectant | Ph. Eur./NF |
| Polysorbate 80 | 1.20 mg | Surfactant | Ph. Eur./USP |
| Water for Injection | q.s. to 6.0 mL | Solvent | Ph. Eur./USP |

¹ Amounts are based on a labeled volume of 6.0 mL/vial.
q.s. = as much as is sufficient.

The excipients comply with the requirements of the applicable compendial monographs (Ph. Eur., USP/NF, JP, ChP). See Table 4. The excipients and their possible stabilizing roles in the formulation of the drug product are summarized in Table 3. No incompatibility is observed between/among these excipients in the formulation and the active drug when tested.

TABLE 4
Excepients in the Drug Product Formulation

| Excipient | Function | Quality Standard |
|---|---|---|
| L-Histidine | Buffer | Ph. Eur./USP |
| L-Histidine monohydrochloride | Buffer | Ph. Eur. |
| monohydrate | | |
| Sucrose | Cryoprotectant | Ph. Eur./NF |
| Polysorbate 80 | Surfactant | Ph. Eur./USP |

ARX305 Drug Product, manufactured under current Good Manufacturing Practices (cGMP), was evaluated in accordance with International Conference on Harmonisation (ICH) guidelines for long term stability at 5° C.±3° C., accelerated stability at 25° C.±2° C./60% relative humidity (RH) and under stressed conditions of 40° C./75% RH. Stability acceptance criteria included cake appearance; moisture content; reconstitution time; clarity after reconstitution; pH after reconstitution; osmolality; protein content (protein concentration); potency (binding ELISA and cell-based assays) and purity (including unconjugated mAb, DAR, free drug, CE-SDS, SEC, non-reduced and reduced capillary electrophoresis sodium dodecyl sulfate (CE-SDS), which measures sample purity as the sum of % heavy chain (HC) and % light chain (LC) under reducing conditions, and cation ion exchange chromatography (CEX)) measures, and safety measures (endotoxin, sterility and sub-visible particulates). For tests that required reconstitution prior to analysis, the lyophilized product (60 mg/vial) was reconstituted with water for injection (WFI; 6 mL).

Long-Term Stability. ARX305 Drug Product exhibited the following characteristics under the long-term stability conditions of 5° C.±3° C. (stored upright) over a period of 24 months, with all tests performed at zero, 1, 3, 6, 9, 12, 18 and 24 months, unless otherwise indicated: appearance prior to reconstitution was a white cake, with no meltback or collapse; clarity after reconstitution was 4.1 to 4.6 nephelometric turbidity units (NTU); reconstituted product was essentially free of visible particles; sub-visible particles (≥10 micron) after reconstitution were <6000 per vial (specifically, 2 to 60 per vial); sub-visible particles (≥25 micron) after reconstitution were <600 per vial (specifically, 0 to 5 per vial); reconstitution time was less than or equal to 10 minutes (specifically, within a range of 1 to 4 minutes); moisture content was less than or equal to 3.0% (specifically, 0.8% to 1.0%); pH after reconstitution was within a range of from about 5.2 to about 6.2 (specifically, from about 5.6 to about 5.7); osmolality was within a range of 260-360 mOsmol/kg (specifically, within a range of 288 to 297 mOsmol/kg); protein content was 95 to 102% of labeled amount; purity based on size exclusion chromatograph (SEC) that measured % monomer (% main peak), % high molecular weight (HMW) and % low molecular weight (LMW) species was >99% main peak, 0.4% to 0.7% HMW species and 0.1% to 0.2% LMW species over the duration of the study; purity via reduced CE-SDS was >97%; purity based on non-reduced CD-SDS was >95%; purity based on CEX was >51% main peak, <40% acidic peak and <15% basic peak; drug-to-antibody ratio (DAR) as measured by hydrophobic interaction chromatography (HIC) was 1.9 at all time points; unconjugated antibody based on HIC was 0.4% to 0.6%; free drug was <0.01% (w/w); cell-based assay results were >85% of reference standard; binding ELISA assay results were >90% of reference standard; and endotoxins were <0.03 EU/mg at tested time points (zero, 12 and 24 mo).

Accelerated Stability. ARX305 Drug Product exhibited the following characteristics under accelerated stability conditions at 25° C.±2° C./60% RH±5% RH (stored upright) over a period of 6 months, with all tests performed at zero, 1, 3 and 6 months, unless otherwise indicated: appearance prior to reconstitution was a white cake, with no meltback or collapse; clarity after reconstitution was 4.2 to 4.6 nephelometric turbidity units (NTU); reconstituted product was essentially free of visible particles; sub-visible particles (≥10 micron) after reconstitution were <6000 per vial (specifically, 17 to 62 per vial); sub-visible particles (≥25 micron) after reconstitution were <600 per vial (specifically, 0 to 11 per vial); reconstitution time was less than or equal to 10 minutes (specifically, within a range of 1 to 4 minutes); moisture content was less than or equal to 3.0% (specifically, 0.9% to 1.1%); pH after reconstitution was within a range of from about 5.2 to about 6.2 (specifically, from about 5.6 to about 5.7); osmolality was within a range of 260-360 mOsmol/kg; protein content was 95 to 101% of labeled amount; purity based on size exclusion chromatograph (SEC) that measured % monomer (% main peak), % high molecular weight (HMW) and % low molecular weight (LMW) species was >99% main peak, 0.4% to 0.7% HMW species and 0.1% to 0.2% LMW species over the duration of the study; purity via reduced CE-SDS was >96%; purity based on non-reduced CD-SDS was >95%; purity based on CEX was >53% main peak, <35% acidic peak and <15% basic peak; drug-to-antibody ratio (DAR) as measured by hydrophobic interaction chromatography (HIC) was 1.9 at all time points; unconjugated antibody based on HIC was 0.5%; free drug was <0.01% (w/w); cell-based assay results were >90% of reference standard; binding ELISA assay results were >90% of reference standard; and endotoxins were <0.03 EU/mg at tested time points (zero and 6 mo).

Stressed conditions of 40° C./75% RH. ARX305 Drug Product exhibited the following characteristics under stressed conditions at 40° C.±2° C./75% RH±5% RH (stored upright) over a period of 1 month, with all tests performed at time zero, 1 week, 2 weeks and 1 month, unless otherwise indicated: appearance prior to reconstitution was a white cake, with no meltback or collapse; clarity after reconstitution was 4.4 to 4.6 nephelometric turbidity units (NTU); reconstituted product was essentially free of visible particles; sub-visible particles (≥10 micron) after reconstitution were <6000 per vial (specifically, 14 to 101 per vial); sub-visible particles (≥25 micron) after reconstitution were <600 per vial (specifically, 0 to 11 per vial); reconstitution time was less than or equal to 10 minutes (specifically, within a range of 1 to 3 minutes); moisture content was less than or equal to 3.0% (specifically, 0.9% to 1.1%); pH after reconstitution was within a range of from about 5.2 to about 6.2 (specifically, from about 5.6 to about 5.7); osmolality was within a range of 260-360 mOsmol/kg (specifically, within a range of 290 to 300 mOsmol/kg); protein content was 95 to 101% of labeled amount; purity based on size exclusion chromatograph (SEC) that measured % monomer (% main peak), % high molecular weight (HMW) and % low molecular weight (LMW) species was >99% main peak, 0.3% to 0.7% HMW species and 0.1% to 0.2% LMW species over the duration of the study; purity via reduced CE-SDS was >97%; purity based on non-reduced CD-SDS was >95%; purity based on CEX was >53% main peak, <40% acidic peak and <15% basic peak; drug-to-antibody ratio (DAR) as measured by hydrophobic interaction chromatography (HIC) was 1.9 at all time points; unconjugated antibody based on HIC was 0.5%; free drug was <0.01% (w/w); cell-based assay results were >90% of reference standard; binding ELISA assay results were ≥100% of reference standard; and endotoxins were ≤0.03 EU/mg at tested time points (zero and 1 mo).

In summary, the stability results showed that ARX305 Drug Product is stable for at least 24 months at 5° C.±3° C., at least 6 months at accelerated condition of 25° C.±2° C./60% RH±5% RH, and at least 1 month at stressed conditions of 40° C./75% RH±5% RH.

### Example 20: Combination Therapy Using ARX305 and Pembrolizumab

ARX305 in combination with a checkpoint inhibitor, such as a PD-1/PD-L1 inhibitor, were tested in a renal cell carcinoma (RCC) model. For RCC, ARX305 in combination of pembrolizumab (PD-1 inhibitor) were evaluated in a cell line-derived xenograft tumor model: 786-OS3. The study design is shown in Table 5.

TABLE 5
Anti-Tumor Activity Testing of Combined ARX305 and Pembrolizumab
(PD-1 inhibitor) in the 786-OS3 CDX Model

| | | Dose | | | Number of |
|---|---|---|---|---|---|
| Group | Treatment | (mg/kg) | Route | Schedule | Subject |
| A | Vehicle | — | IV | QW | 10 |
| B | ARX305 | 0.3 | IV | Q2W × 2 | 10 |
| C | Pembrolizumab | 5 | IP | QW × 4 | 10 |
| D | ARX305 + | 0.3/5 | IV/IP | Q2W × 2/ | 10 |
| | Pembrolizumab | | | QW × 4 | |

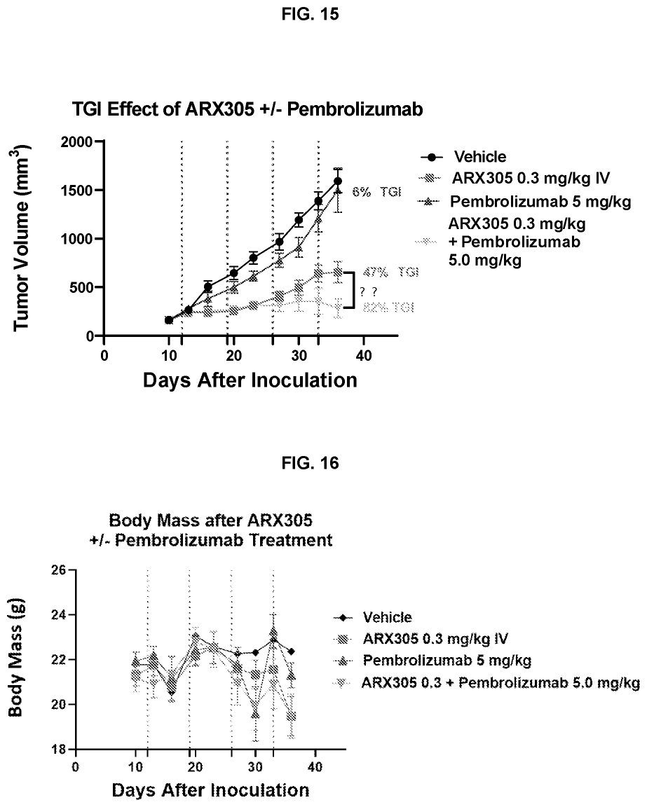

Female NCG mice were housed five (5) per cage in a pathogen-free, limited access room. Food and water were provided ad libitum. Mice were subcutaneously inoculated in the right flank with 5×10⁶ 786-OS3 renal carcinoma cells mixed with Matrigel. Tumor volumes were measured twice weekly. When tumors reached from about 175 mm³ to about 200 mm³ in size, mice were randomized into five (5) groups of 10 mice with approximately equal average tumor volumes. Cryopreserved peripheral blood mononuclear cells (PBMCs) from a single healthy donor were thawed and cultured in Roswell Park Memorial Institut (RPMI) medium+10% fetal bovine serum at 37° C. for 24 hours. PBMCs were harvested and intraperitoneally injected into tumor-bearing mice (10⁷/mouse). After 24 hours, mice were injected with vehicle, pembrolizumab (5 mg/kg), ARX305 (0.3 mg/kg), or pembrolizumab (5 mg/kg)+ARX305 (0.3 mg/kg). Dosing was repeated for pembrolizumab weekly for a total of 4 doses and ARX305 every other week for a total of 2 doses. About 10⁷ healthy donor human PBMC transfer was repeated weekly as described above (injected QW×4); injections were coincident with the pembrolizumab administration. Tumor volumes were continually measured twice weekly and FIG. 15 show mean tumor volumes +/−SEM for each group.

The combination of ARX305 pembrolizumab resulted in strong anti-tumor activity in the 786-OS3 RCC xenograft model and exhibited increased anti-tumor activity in combination vs. single test article administration. FIG. 15 shows ARX305 and pembrolizumab demonstrating additive tumor growth inhibition (TGI). For example, on study day 36, 6% TGI was observed at 5.0 mg/kg dose of pembrolizumab alone; 47% TGI was observed at 0.3 mg/kg dose of ARX305 alone; but 82% TGI was observed for the combination of the 0.3 mg/kg dose of ARX305 and 5.0 mg/kg dose of pembrolizumab. To evaluate the safety of the combination therapy, body weight of each test subject was measured and recorded periodically. FIG. 16 shows that the combination of ARX305 and pembrolizumab did not induce major body weight changes in the 786-OS3/PBMC model, thereby indicating this combination is safe.

It is understood that the examples and embodiments described herein are for illustrative purposes only and that various modifications or changes in light thereof will be suggested to those of ordinary skill in the art and are to be included within the spirit and purview of this application and scope of the appended claims. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. An antibody-drug conjugate (ADC) comprising:
one or more drug-linker groups having the structure: 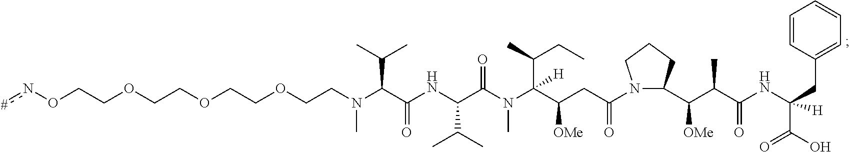
and
an anti-CD70 antibody or fragment thereof comprising one or more heavy chains, wherein at least one of the one or more heavy chains comprises an amino acid sequence containing a first non-naturally encoded amino acid, wherein the amino acid sequence comprises SEQ ID NO: 3, 4 or 5;
wherein:
each [symbol] represents a single bond or a double bond that covalently joins one of the one or more drug-linker groups to the anti-CD70 antibody or fragment thereof; and
each #represents a site of connection to the anti-CD70 antibody or fragment thereof.

2. (canceled)

3. The ADC of claim 1, wherein the anti-CD70 antibody or fragment thereof further comprises one or more light chains, wherein at least one of the one or more light chains comprises an amino acid sequence that shares at least 90% identity with SEQ ID NO: 2, 6, 7, 8 or 9.

4. (canceled)

5. The ADC of claim 4, wherein at least one of the one or more light chains comprises an amino acid sequence containing a second non-naturally encoded amino acid, wherein the amino acid sequence comprises SEQ ID NO: 6, 7, 8 or 9.

6.-10. (canceled)

11. The ADC of claim 1, wherein the ADC is an ADC of Formula (I): 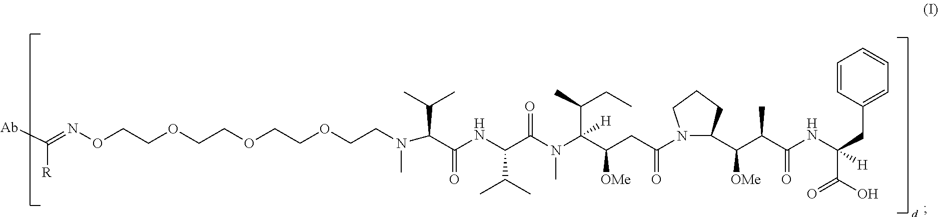
wherein:
Ab is the anti-CD70 antibody or fragment thereof;
each R is independently unsubstituted C₁-C₅ alkyl; and
d is an integer of 1-10.

12.-16. (canceled)

17. The ADC of claim 11, wherein the anti-CD70 antibody or fragment thereof is a humanized monoclonal antibody comprising two heavy chains and two light chains, wherein each heavy chain comprises the amino acid sequence of SEQ ID NO: 3, and each light chain comprises the amino acid sequence of SEQ ID NO: 2.

18. (canceled)

19. (canceled)

20. The ADC of claim 11, wherein:
the anti-CD70 antibody or fragment thereof is a humanized anti-CD70 monoclonal antibody comprising two heavy chains, two light chains, and two non-naturally encoded amino acids, wherein:
the amino acid sequence of each of the two heavy chains is SEQ ID NO: 3, wherein SEQ ID NO: 3 comprises one non-naturally encoded amino acid, wherein the one non-naturally encoded amino acid in SEQ ID NO: 3 is para-acetyl-L-phenylalanine at position A114 (Kabat numbering) of SEQ ID NO: 3; and
the amino acid sequence of each of the light chains is SEQ ID NO: 2;
each R is methyl;
the one or more drug-linker groups is two drug-linker groups; and
d is 2;
wherein each of the two drug-linker groups is connected to each para-acetyl-L-phenylalanine at position A114 of SEQ ID NO: 3, thereby connecting each of the two drug-linker groups to the humanized anti-CD70 monoclonal antibody.

21. A composition comprising an ADC of claim 1.

22.-24. (canceled)

25. The composition of claim 21, wherein the composition comprising the ADC is a pharmaceutical composition comprising an effective amount of the ADC, and the pharmaceutical composition further comprises a pharmaceutically acceptable excipient.

26.-28. (canceled)

29. A method of treating a disease or condition in a subject in need thereof, the method comprising administering to the subject an effective amount of an ADC of claim 1,
wherein the disease or condition is a tumor or cancer,
wherein the tumor or cancer is: (i) a solid tumor: (ii) a hematological cancer: (iii) kidney cancer, brain cancer, breast cancer, Burkitt's lymphoma, ovarian cancer, gastric cancer, gastroesophageal junction adenocarcinoma, cervical cancer, uterine cancer, endometrial cancer, testicular cancer, prostate cancer, colorectal cancer, esophageal cancer, bladder cancer, lung cancer, non-small cell lung cancer, urothelial carcinoma, cholangiocarcinoma, colon biliary tract cancer, pancreatic cancer, renal cell cancer, nasopharyngeal cancer, Mantle cell lymphoma, multiple myeloma, non-Hodgkin's lymphoma, Hodgkin's lymphoma or acute myeloid leukemia; (iv) a multi-drug resistant tumor or cancer: (v) a CD70-expressing cancer, or (vi) resistant or refractory to prior standard therapies.

30.-39. (canceled)

40. The method of claim 29, further comprising treating the subject with an effective amount of an additional therapeutic agent,
wherein the additional therapeutic agent is
(i) a chemotherapeutic agent, a hormonal agent, an antitumor agent, an immunostimulatory agent, an immunomodulator or an immunotherapeutic agent; or a combination thereof;
(ii) a checkpoint inhibitor, CD70 kinase inhibitor, cyclin-dependent kinase inhibitor, tyrosine kinase inhibitor, small-molecule kinase inhibitor, hypomethylating agent or platinum-based therapeutic: or a combination thereof;
(iii) a PD-1 inhibitor, wherein the PD-1 inhibitor is AMP-224, atezolizumab, avelumab, BMS-936558, BMS-936559, CT-001, durvalumab, MEDI0680, nivolumab, PDR001, pembrolizumab, pidilizumab or REGN2810; or
(iv) a radiotherapy.

41.-47. (canceled)

48. The method of claim 29, wherein the CD70-expressing cancer has at least about 5,000 CD70 molecules/cell, at least about 10,000 CD70 molecules/cell, or at least about 15,000 CD70 molecules/cell.

49.-56. (canceled)

57. The method of claim 29, wherein the effective amount of the ADC is a dose within a range of about 0.05 mg/kg to about 10 mg/kg, about 0.05 mg/kg to about 2 mg/kg, about 2 mg/kg to about 5 mg/kg, about 5 mg to about 10 mg/kg of the human subject, or any value in between.

58.-82. (canceled)

83. A formulation comprising an ADC of claim 1.

84. The formulation of claim 83, further comprising a histidine buffer, a sucrose cryoprotectant, and/or a polysorbate 80 surfactant.

85.-89. (canceled)

90. The formulation of claim 83, wherein the formulation is a lyophilized drug product.

91. A reconstituted solution comprising a formulation of claim 90 and a diluent, wherein the reconstituted solution comprises the ADC at a concentration within a range of about 5 mg/mL to about 25 mg/mL, and wherein the reconstituted solution pH is less than 6, and wherein the diluent is water.

92. The reconstituted solution of claim 91, wherein the histidine buffer is L-histidine buffer at a concentration within a range of about 10 mM to about 50 mM; the sucrose is at a concentration within a range of about 1% (w/v) to about 20% (w/v); and the polysorbate 80 is at a concentration within a range of about 0.01% (w/v) to about 0.1% (w/v), and wherein the reconstituted solution has a pH within a range of about 5.2 to about 6.2.

93. (canceled)

94. (canceled)

95. A formulation comprising an ADC of claim 1 from about 5 mg/mL to about 15 mg/mL; histidine buffer from about 15 mM to about 25 mM; sucrose from about 5% (w/v) to about 15% (w/v); and polysorbate 80 from about 0.01% (w/v) to about 0.05% (w/v); wherein the formulation is at a pH from about 5.4 to about 6.0.

96. The formulation of claim 95, comprising the ADC at about 10 mg/mL; histidine buffer at about 20 mM, sucrose at about 8% (w/v); and polysorbate 80 at about 0.02% (w/v); wherein the formulation pH is about 5.7.

97. The method of claim 29-68, wherein the administering of the effective amount of the ADC is performed on a dosing schedule, wherein the dosing schedule is: (i) once every 1, 2, 3, 4, 5, or 6 weeks: (ii) more than once within a three-week cycle; (iii) at least once every four weeks for at least about eight weeks: or (iv) once every three weeks for at least about eight weeks.

98.-104. (canceled)


SEQUENCE LISTING (9 sequences)

SEQ ID NO: 1 - protein, 447 aa, synthetic construct
EVQLVQSGAEVKKPGATVKISCKVSGYTFTDYYMNWVQQAPGKGLEWMGIINPYNGGTHYNQKFKGRVTITADTSTDTAYMELSSLRSEDTAVYYCATSGYDLYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, synthetic construct
  MOD_RES 114: note = non-natural amino acid
EVQLVQSGAEVKKPGATVKISCKVSGYTFTDYYMNWVQQAPGKGLEWMGIINPYNGGTHYNQKFKGRVTITADTSTDTAYMELSSLRSEDTAVYYCATSGYDLYFDYWGQGTLVTVSSXSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 447 aa, synthetic construct
  MOD_RES 136: note = non-natural amino acid
EVQLVQSGAEVKKPGATVKISCKVSGYTFTDYYMNWVQQAPGKGLEWMGIINPYNGGTHYNQKFKGRVTITADTSTDTAYMELSSLRSEDTAVYYCATSGYDLYFDYWGQGTLVTVSSASTKGPSVFPLAPSSKSTSGGTXALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 447 aa, synthetic construct
  MOD_RES 159: note = non-natural amino acid
EVQLVQSGAEVKKPGATVKISCKVSGYTFTDYYMNWVQQAPGKGLEWMGIINPYNGGTHYNQKFKGRVTITADTSTDTAYMELSSLRSEDTAVYYCATSGYDLYFDYWGQGTLVTVSSASTKGPSVFPLAPSSKSTSGGTAALGCLVKDYFPEPVTVSWNSGAXTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 214 aa, synthetic construct
  MOD_RES 110: note = non-natural amino acid
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTXAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 214 aa, synthetic construct
  MOD_RES 112: note = non-natural amino acid
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTVAXPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 214 aa, synthetic construct
  MOD_RES 114: note = non-natural amino acid
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTVAAPXVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 214 aa, synthetic construct
  MOD_RES 121: note = non-natural amino acid
EIVMTQSPATLSVSPGERATLSCKASQNVGTAVAWYQQKPGQAPRLLIYSAFNRYNGIPARFSGSGPGTDFTLTISSLQSEDFAVYYCQQYSTYPLTFGGGTKVEIKRTVAAPSVFIFPPXDEQLKSGTASVVCLLNNFYPREAKVQWKVDNALQSGNSQESVTEQDSKDSTYSLSSTLTLSKADYEKHKVYACEVTHQGLSSPVTKSFNRGEC